(19) United States
(12) Patent Application Publication
(10) Pub. No.: US 2024/0084340 A1
(43) Pub. Date: Mar. 14, 2024

(54) HETEROLOGOUS PRODUCTION OF 10-METHYLSTEARIC ACID

(71) Applicant: Ginkgo Bioworks, Inc., Boston, MA (US)

(72) Inventors: Arthur J. Shaw, Belmont, MA (US); Hannah Blitzblau, Arlington, MA (US); Donald V. Crabtree, Cambridge, MA (US)

(21) Appl. No.: 18/227,147

(22) Filed: Jul. 27, 2023

Related U.S. Application Data
(63) Continuation of application No. 17/197,710, filed on Mar. 10, 2021, now abandoned.
Application No. 17/197,710 is a continuation of application No. 16/664,378, filed on Oct. 25, 2019, now Pat. No. 10,975,398.
Application No. 16/664,378 is a division of application No. 15/710,734, filed on Sep. 20, 2017, now Pat. No. 10,457,963.
(60) Provisional application No. 62/396,870, filed on Sep. 20, 2016.

(51) Int. Cl. C12P 7/6409; A23D 9/02; C11B 1/10; C11C 3/00; C12N 9/02; C12N 9/10; C12Q 1/686

(52) U.S. Cl. CPC C12P 7/6409; A23D 9/02; C11B 1/10; C11C 3/00; C12N 9/001; C12N 9/1007; C12Q 1/686

(57) ABSTRACT

Nucleic acids and cells comprising a methyltransferase gene and/or a reductase gene are disclosed. These nucleic acids and cells may be used to produce branched (methyl)lipids, such as 10-methylstearate.

### CROSS REFERENCE TO RELATED APPLICATIONS

This application is a continuation of U.S. patent application Ser. No. 17/197,710, filed Mar. 10, 2021, which is a continuation of U.S. patent application Ser. No. 16/664,378, filed Oct. 25, 2019, now U.S. Pat. No. 10,975,398, which is a divisional of U.S. patent application Ser. No. 15/710,734, filed Sep. 20, 2017, now U.S. Pat. No. 10,457,963, which claims priority to U.S. Provisional Patent Application Ser. No. 62/396,870, filed Sep. 20, 2016, each of which are incorporated by reference herein in their entirety.

### REFERENCE TO A SEQUENCE LISTING XML

This application contains a Sequence Listing which has been submitted electronically in XML format. The Sequence Listing XML is incorporated herein by reference. Said XML file, created on Nov. 17, 2023, is named GBB-74104_SL.xml and is 297,774 in size.

### BACKGROUND

Fatty acids derived from agricultural plant and animal oils find use as industrial lubricants, hydraulic fluids, greases, and other specialty fluids in addition to oleochemical feedstocks for processing. The physical and chemical properties of these fatty acids result in large part from their carbon chain length and number of unsaturated double bonds. Fatty acids are typically 16:0 (sixteen carbons, zero double bonds), 16:1 (sixteen carbons, 1 double bond), 18:0, 18:1, 18:2, or 18:3. Importantly, fatty acids with no double bonds (saturated) have high oxidative stability, but they solidify at low temperature. Double bonds improve low-temperature fluidity, but decrease oxidative stability. This trade-off poses challenges for lubricant and other specialty-fluid formulations because consistent long term performance (high oxidative stability) over a wide range of operating temperatures is desirable. High 18:1 (oleic) fatty acid oils provide low temperature fluidity with relatively good oxidative stability. Accordingly, several commercial products, such as high oleic soybean oil, high oleic sunflower oil, and high oleic algal oil, have been developed with high oleic compositions. Oleic acid is an alkene, however, and subject to oxidative degradation.

### SUMMARY

The nucleic acids, cells, and methods described herein are generally useful for the production of branched (methyl)lipids, such as 10-methylstearic acid, and compositions that include such lipids. Saturated branched (methyl)lipids like 10-methylstearic acid have favorable low-temperature fluidity and favorable oxidative stability, which are desirable properties for lubricants and specialty fluids.

Various aspects relate to nucleic acids comprising a recombinant tmsB gene encoding a methyltransferase protein, a recombinant tmsA gene encoding a reductase protein, and/or a recombinant tmsC gene encoding a tmsC protein. The methyltransferase protein, reductase protein, and/or tmsC protein may be proteins expressed by species of Actinobacteria, and the recombinant tmsB gene, recombinant tmsA gene, and/or recombinant tmsC gene may be codon-optimized for expression in a different phylum of bacteria (e.g., Proteobacterium) or in eukaryotes (e.g., yeast, such as Arxula adeninivorans (also known as Blastobotrys adeninivorans or Trichosporon adeninivorans), Saccharomyces cerevisiae, or Yarrowia lipolytica). The recombinant tmsB gene, recombinant tmsA gene, or recombinant tmsC gene may be operably-linked to a promoter capable of driving expression in a phylum of bacteria other than Actinobacteria (e.g., Proteobacterium) or in eukaryotes (e.g., yeast). The nucleic acid may be a plasmid or a chromosome.

Some aspects relate to a cell comprising a nucleic acid as described herein. The cell may comprise a branched (methyl)lipid, such as 10-methylstearic acid, and/or an exomethylene-substituted lipid, such as 10-methylenestearic acid. The cell may be a eukaryotic cell, such as an algae cell, yeast cell, or plant cell.

Some aspects relate to a composition produced by cultivating a cell culture comprising cells as described herein. The oil composition may comprise a branched (methyl)lipid, such as 10-methylstearic acid, and or an exomethylene-substituted lipid, such as 10-methylenestearic acid.

### BRIEF DESCRIPTION OF THE DRAWINGS

FIG. 1 depicts one possible mechanism for the conversion of oleic acid to 10-methylstearic acid. An oleic acid substrate may be present as an acyl chain of a glycerolipid or phospholipid. A methionine substrate, which donates the methyl group, may be present as S-adenosyl methionine. The oleic acid and methionine substrates may be converted to 10-methylenestearic acid (e.g., present as an acyl chain of a glycerolipid or phospholipid) and homocysteine (e.g., present as S-adenosyl homocysteine). This reaction may be catalyzed by a tmsB protein as described herein, infra. 10-methylenestearic acid (e.g., present as an acyl chain of a glycerolipid or phospholipid) may be reduced to 10-methylstearic acid. The reduction may be catalyzed by a tmsA protein as describe herein, infra, for example, using NADPH as a reducing agent. The language of the specification and claims, however, is not limited to any particular reaction mechanism.

FIG. 2 depicts one possible mechanism for the conversion of oleic acid to 10-methylstearic acid. Oleic acid, present as a carboxylic acid in the cytosol, may be added to monoacylglycerol-3-phosphate to form a diacylglycerol-3-phosphate comprising an oleate acyl group. “10-methyl synthase” may convert diacylglycerol-3-phosphate comprising an oleate acyl group to diacylglycerol-3-phosphate comprising a 10-methylsterate acyl group. The diacyl-3-phosphate may subsequently be converted to a triacylglycerol, converted into another phospholipid, such as phosphatidylcholine, or converted back into a monoacylglycerol-3-phosphate (e.g., thereby releasing free 10-methylstearate into the cytosol). The language of the specification and claims, however, is not limited to any particular reaction mechanism.

FIG. 3 depicts prokaryotic operons encoding enzymes that catalyze the transfer of methyl groups to alkyl chains from sixteen different species of bacteria, labeled A-P. The tmsA and tmsB genes are particularly important for methylating alkyl chains. The tmsC gene may also be important for methylating alkyl chains. The nucleotide sequences of these genes and the amino acid sequences that they encode are shown in SEQ ID NO:1-76.

FIG. 4 is a map of plasmid pNC704, which may be used to express Mycobacterium smegmatis genes tmsA (SEQ ID NO:1) and tmsB (SEQ ID NO:3) in E. coli. The nucleotide sequence of plasmid pNC738 is set forth in SEQ ID NO:77.

FIG. 5 is a map of plasmid pNC738, which may be used to express codon-optimized versions of Mycobacterium smegmatis genes tmsA (SEQ ID NO:80) and tmsB (SEQ ID NO:81) in yeast, such as Arxula adeninivorans, Saccharomyces cerevisiae, and Yarrowia lipolytica. The nucleotide sequence of plasmid pNC738 is set forth in SEQ ID NO:78.

FIG. 6 is a map of plasmid BS-10MS_ER, which may be used to express codon-optimized versions of Mycobacterium smegmatis genes tmsA (SEQ ID NO:80) and tmsB (SEQ ID NO:81) in yeast, such as Arxula adeninivorans, Saccharomyces cerevisiae, and Yarrowia lipolytica. The nucleotide sequence of plasmid BS-10MS_ER is set forth in SEQ ID NO:79.

FIGS. 7A and 7B consist of overlaid gas chromatography (GC) traces of various fatty acid standards and lipids extracted from various samples. The standards were stearic acid, 10-methylstearic acid, and oleic acid. Each sample and standard was transesterified into fatty acid methyl esters (FAMEs) prior to analysis. FIG. 7A depicts the GC trace of FAMEs prepared from E. coli that express the tmsA and tmsB genes from Mycobacterium smegmatis as well as the GC traces of each standard. The tmsAltmsB sample displayed a peak at about 10.777 minutes, corresponding to the 10-methylstearic acid standard. FIG. 7B depicts each trace of FIG. 7A and two additional traces. The first additional trace corresponds to FAMEs prepared from E. coli that express the ufa gene from Mycobacterium tuberculosis. This sample displayed a peak at about 10.777 minutes, corresponding to the 10-methylstearic acid standard. The second additional trace corresponds to FAMEs prepared from E. coli that had been transfected with an empty vector. This control did not display a peak at 10.777 minutes, suggesting that the tmsA and tmsB genes synthesized 10-methylstearic acid in the transformed E. coli.

FIGS. 8A and 8B depict GC-MS result. FIG. 8A is a gas chromatography (GC) trace of lipids eluting from a GC column. The lipids were purified from E. coli that had been transfected with pNC704 encoding Mycobacterium smegmatis genes tmsA and tmsB, and the lipids were converted into fatty acid methyl esters. FIG. 8B is a mass spectroscopy spectrum of the lipids eluted during the GC run of panel A from 20.388 to 20.447 minutes. The mass spectrum is gated for the 10-methylstearate fatty acid methyl ester, which has a molecular weight of 312. The spectrum also displays a peak at 313 m/z corresponding to 10-methylstearate methyl esters comprising natural-abundance isotopes (e.g., a single ¹³C).

FIGS. 9A-9D depict maps of the following vectors, which can be used to express the tmsA and tmsB genes of the indicated species: pNC721 (Mycobacterium vanbaaleni) (SEQ ID NO:83), pNC755 (Amycolicicoccus subflavus) (SEQ ID NO:84), pNC757 (Corynebacterium glyciniphilum) (SEQ ID NO:85), pNC 904 (Rhodococcus opacus) (SEQ ID NO:86), pNC905 (Thermobifida fusca) (SEQ ID NO:87), pNC906 (Thermomonospora curvata) (SEQ ID NO:88), pNC907 (Corynebacterium glutamicum) (SEQ ID NO:89), pNC908 (Agromycies subbeticus) (SEQ ID NO:90), pNC910 (Mycobacterium gilvum) (SEQ ID NO:91), pNC911 (Mycobacterium sp. indicus) (SEQ ID NO:92).

FIG. 10 depicts maps of vectors pNC985 (SEQ ID NO:93), which can be used to express the M. smegmatis tmsAB genes in Rhodococcus bacteria, and pNC986 (SEQ ID NO:94), which can be used to express the T. fusca tmsAB genes in Rhodococcus bacteria.

FIG. 11 depicts maps of vectors pNC963 (SEQ ID NO:95), which encodes the T. curvata tmsB gene under control of the constitutive tac promoter, and pNC964 (SEQ ID NO:96), which encodes the T curvata tmsA gene under control of the constitutive tac promoter.

FIG. 12 is a graph showing gas chromatographic detection of 10-methylene stearic acid in Y. lipolytica expressing tmsB genes from various organisms.

FIG. 13 is a graph showing percentage of 10-methylene fatty acids as compared to total fatty acids in 8 transformants of Arxula adeninivorans containing a plasmid encoding T. curvata tmsB. The two isolates furthest to the right were transformed with empty vector control.

FIG. 14 is a graph showing the percentage by weight of 10-methylene fatty acids and 10-methyl fatty acids in Yarrowia lipolytica containing a stably integrated copy of the T. curvata tmsB gene and transformed with plasmids expressing tmsA from C. glutamicum (C.gl.), T. curvata (T.cu.), or T. fusca (T.fu.), or an empty vector control (the two transformants furthest to the right).

FIG. 15 is a graph showing the percentage by weight of 10-methylene fatty acids and 10-methyl fatty acids as compared to total fatty acids in transformants of S. cerevisiae transformed with empty vector (-) or vectors encoding T. curvata tmsA (T.cu. tmsA), T. curvata tmsB (T.cu. tmsB), or both T. curvata tmsA and tmsB (T.cu. tmsA+tmsB).

FIG. 16 is a graph showing the percentage by weight of 10-methylene fatty acids and 10-methyl fatty acids as compared to total fatty acids in transformants of S. cerevisiae containing the tmsA-B fusion protein, the tmsB-A fusion protein, or empty vector (-).

FIG. 17 is a graph showing the percentage by weight of 10-methylene fatty acids and 10-methyl fatty acids as compared to total fatty acids in transformants of Y. lipolytica containing the tmsA-B fusion protein, the tmsB-A fusion protein, or empty vector (-).

FIG. 18 is a graph showing the percentage by weight of 10-methylene fatty acids and 10-methyl fatty acids as compared to total fatty acids in transformants of A. adeninivorans containing the tmsA-B fusion protein or empty vector (-).

FIGS. 19A-19D show a CLUSTAL OMEGA alignment of TmsB protein sequences encoded by the tmsB genes from Mycobacterium smegmatis (SEQ ID NO:4), Mycobacterium vanbaaleni (SEQ ID NO:54), Amycolicicoccus subflavus (SEQ ID NO:12), Corynebacterium glyciniphilum (SEQ ID NO:20), Corynebacterium glutamicum (SEQ ID NO:16), Rhodococcus opacus (SEQ ID NO:60), Agromyces subbeticus (SEQ ID NO:8), Knoellia aerolata (SEQ ID NO:26), Mycobacterium gilvum (SEQ ID NO:36), Mycobacterium sp. Indicus (SEQ ID NO:42), Thermobifida fusca (SEQ ID NO:70), and Thermomonospora curvata (SEQ ID NO:76), along with the cyclopropane fatty acid synthase (Cfa) enzyme from Escherichia coli (SEQ ID NO: 100).

FIGS. 20A-20E show a CLUSTAL OMEGA alignment of TmsA protein sequences encoded by the tmsA genes from Mycobacterium smegmatis (SEQ ID NO:2), Mycobacterium vanbaaleni (SEQ ID NO:52), Amycolicicoccus subflavus (SEQ ID NO:10), Corynebacterium glyciniphilum (SEQ ID NO:18), Corynebacterium glutamicum (SEQ ID NO:14), Rhodococcus opacus (SEQ ID NO:58), Agromyces subbeticus (SEQ ID NO:6), Knoellia aerolata (SEQ ID NO:24), Mycobacterium gilvum (SEQ ID NO:34), Mycobacterium sp. Indicus (SEQ ID NO:40), Thermobifida fusca (SEQ ID NO:68), and Thermomonospora curvata (SEQ ID NO:74), along with the Glycolate oxidase subunit GlcD enzyme from Escherichia coli (SEQ ID NO: 101).

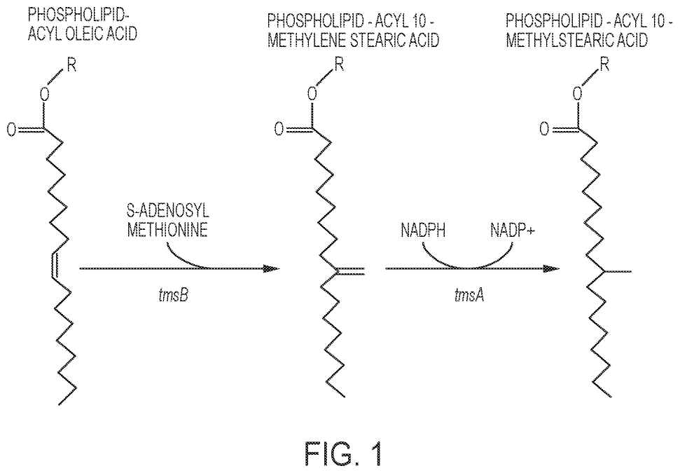

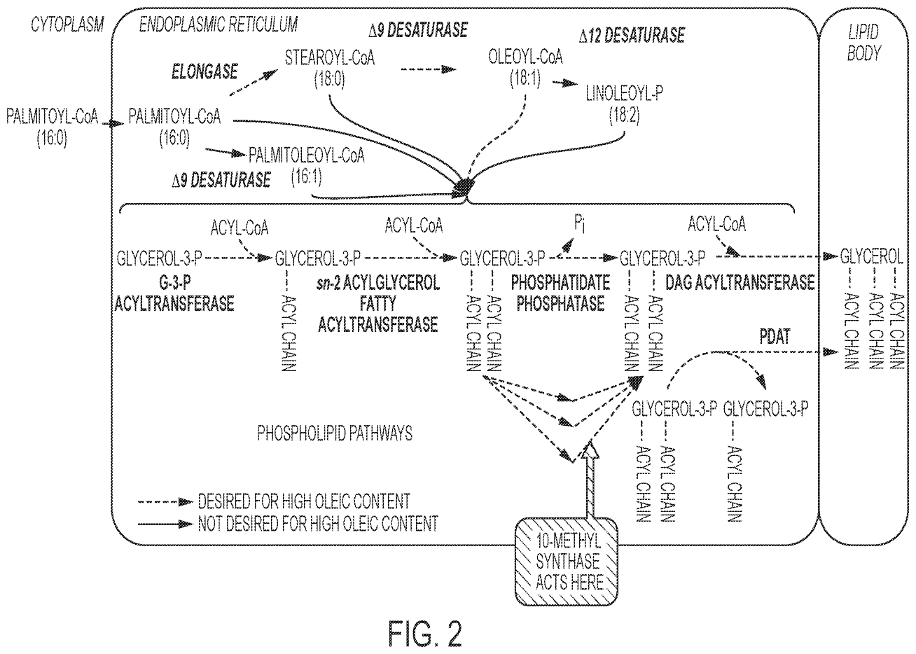

### DETAILED DESCRIPTION

### Definitions

The articles “a” and “an” are used herein to refer to one or to more than one (i.e., to at least one) of the grammatical object of the article. By way of example, “an element” means one element or more than one element.

The term “biologically-active portion” refers to an amino acid sequence that is less than a full-length amino acid sequence, but exhibits at least one activity of the full length sequence. For example, a biologically-active portion of a methyltransferase may refer to one or more domains of tmsB having biological activity for converting oleic acid (e.g., a phospholipid comprising an ester of oleate) and methionine (e.g., S-adenosyl methionine) into 10-methylenestearic acid (e.g., a phospholipid comprising an ester of 10-methylenestearate). A biologically-active portion of a reductase may refer to one or more domains of tmsA having biological activity for converting 10-methylenestearic acid (e.g., a phospholipid comprising an ester of 10-methylenestearate) and a reducing agent (e.g., NADH, NADPH, FAD, FADH2, FMNH2) into 10-methylstearic acid (e.g., a phospholipid comprising an ester of 10-methylstearate). Biologically-active portions of a protein include peptides or polypeptides comprising amino acid sequences sufficiently identical to or derived from the amino acid sequence of the protein, e.g., the amino acid sequence set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, or 76, which include fewer amino acids than the full length protein, and exhibit at least one activity of the protein, especially methyltransferase or reductase activity. A biologically-active portion of a protein may comprise, comprise at least, or comprise to most, for example, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more amino acids or any range derivable therein. Typically, biologically-active portions comprise a domain or motif having a catalytic activity, such as catalytic activity for producing 10-methylenestearic acid or 10-methylstearic acid. A biologically-active portion of a protein includes portions of the protein that have the same activity as the full-length peptide and every portion that has more activity than background. For example, a biologically-active portion of an enzyme may have, have at least, or have at most 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, 400% or higher activity relative to the full-length enzyme (or any range derivable therein). A biologically-active portion of a protein may include portions of a protein that lack a domain that targets the protein to a cellular compartment.

The terms “codon optimized” and “codon-optimized for the cell” refer to coding nucleotide sequences (e.g., genes) that have been altered to substitute at least one codon that is relatively rare in a desired host cell with a synonymous codon that is relatively prevalent in the host cell. Codon optimization thereby allows for better utilization of the tRNA of a host cell by matching the codons of a recombinant gene with the tRNA of the host cell. For example, the codon usage of the species of Actinobacteria (prokaryotes) varies from the codon usage of yeast (eukaryotes). The translation efficiency in a yeast host cell of an mRNA encoding a Actinobacteria protein may be increased by substituting the codons of the corresponding Actinobacteria gene with codons that are more prevalent in the particular species of yeast. A codon optimized gene thereby has a nucleotide sequence that varies from a naturally-occurring gene.

The term “constitutive promoter” refers to a promoter that mediates the transcription of an operably linked gene independent of a particular stimulus (e.g., independent of the presence of a reagent such as isopropyl β-D-1-thiogalactopyrano side).

The term “DGAT1” refers to a gene that encodes a type 1 diacylglycerol acyltransferase protein, such as a gene that encodes a yeast DGA2 protein.

The term “DGAT2” refers to a gene that encodes a type 2 diacylglycerol acyltransferase protein, such as a gene that encodes a yeast DGA1 protein.

“Diacylglyceride,” “diacylglycerol,” and “diglyceride,” are esters comprised of glycerol and two fatty acids.

The terms “diacylglycerol acyltransferase” and “DGA” refer to any protein that catalyzes the formation of triacylglycerides from diacylglycerol. Diacylglycerol acyltransferases include type 1 diacylglycerol acyltransferases (DGA2), type 2 diacylglycerol acyltransferases (DGA1), and type 3 diacylglycerol acyltransferases (DGA3) and all homologs that catalyze the above-mentioned reaction.

The terms “diacylglycerol acyltransferase, type 1” and “type 1 diacylglycerol acyltransferases” refer to DGA2 and DGA2 orthologs.

The terms “diacylglycerol acyltransferase, type 2” and “type 2 diacylglycerol acyltransferases” refer to DGA1 and DGA1 orthologs.

The term “domain” refers to a part of the amino acid sequence of a protein that is able to fold into a stable three-dimensional structure independent of the rest of the protein.

The term “drug” refers to any molecule that inhibits cell growth or proliferation, thereby providing a selective advantage to cells that contain a gene that confers resistance to the drug. Drugs include antibiotics, antimicrobials, toxins, and pesticides.

“Dry weight” and “dry cell weight” mean weight determined in the relative absence of water. For example, reference to oleaginous cells as comprising a specified percentage of a particular component by dry weight means that the percentage is calculated based on the weight of the cell after substantially all water has been removed. The term “% dry weight,” when referring to a specific fatty acid (e.g., oleic acid or 10-methylstearic acid), includes fatty acids that are present as carboxylates, esters, thioesters, and amides. For example, a cell that comprises 10-methylstearic acid as a percentage of total fatty acids by % dry cell weight includes 10-methylstearic acid, 10-methylstearate, the 10-methylstearate portion of a diacylglycerol comprising a 10-methylstearate ester, the 10-methylstearate portion of a triacylglycerol comprising a 10-methylstearate ester, the 10-methylstearate portion of a phospholipid comprising a 10-methylstearate ester, and the 10-methylstearate portion of 10-methylstearate CoA. The term “% dry weight,” when referring to a specific type of fatty acid (e.g., C16 fatty acids, C18 fatty acids), includes fatty acids that are present as carboxylates, esters, thioesters, and amides as described above (e.g., for 10 methylstearic acid).

The term “encode” refers to nucleic acids that comprise a coding region, portion of a coding region, or compliments thereof. Both DNA and RNA may encode a gene. Both DNA and RNA may encode a protein.

The term “enzyme” as used herein refers to a protein that can catalyze a chemical reaction.

The term “expression” refers to the amount of a nucleic acid or amino acid sequence (e.g., peptide, polypeptide, or protein) in a cell. The increased expression of a gene refers to the increased transcription of that gene. The increased expression of an amino acid sequence, peptide, polypeptide, or protein refers to the increased translation of a nucleic acid encoding the amino acid sequence, peptide, polypeptide, or protein.

The term “gene,” as used herein, may encompass genomic sequences that contain exons, particularly polynucleotide sequences encoding polypeptide sequences involved in a specific activity. The term further encompasses synthetic nucleic acids that did not derive from genomic sequence. In certain embodiments, the genes lack introns, as they are synthesized based on the known DNA sequence of cDNA and protein sequence. In other embodiments, the genes are synthesized, non-native cDNA wherein the codons have been optimized for expression in Y. lipolytica or A. adeninivorans based on codon usage. The term can further include nucleic acid molecules comprising upstream, downstream, and/or intron nucleotide sequences.

The term “inducible promoter” refers to a promoter that mediates the transcription of an operably linked gene in response to a particular stimulus.

The term “integrated” refers to a nucleic acid that is maintained in a cell as an insertion into the cell's genome, such as insertion into a chromosome, including insertions into a plastid genome.

“In operable linkage” refers to a functional linkage between two nucleic acid sequences, such a control sequence (typically a promoter) and the linked sequence (typically a sequence that encodes a protein, also called a coding sequence). A promoter is in operable linkage with a gene if it can mediate transcription of the gene.

The term “knockout mutation” or “knockout” refers to a genetic modification that prevents a native gene from being transcribed and translated into a functional protein.

The term “nucleic acid” refers to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “phospholipid” refers to esters comprising glycerol, two fatty acids, and a phosphate. The phosphate may be covalently linked to carbon-3 of the glycerol and comprise no further substitution, i.e., the phospholipid may be a phosphatidic acid. The phosphate may be substituted with ethanolamine (e.g., phosphatidylethanolamine), choline (e.g., phosphatidylcholine), serine (e.g., phosphatidylserine), inositol (e.g., phosphatidylinositol), inositol phosphate (e.g., phosphatidylinositol-3-phosphate, phosphatidylinositol-4-phosphate, phosphatidylinositol-5-phosphate), inositol bisphosphate (e.g., phosphatidylinositol-4,5-bisphosphate), or inositol triphosphate (e.g., phosphatidylinositol-3,4,5-bisphosphate).

As used herein, the term “plasmid” refers to a circular DNA molecule that is physically separate from an organism's genomic DNA. Plasmids may be linearized before being introduced into a host cell (referred to herein as a linearized plasmid). Linearized plasmids may not be self-replicating, but may integrate into and be replicated with the genomic DNA of an organism.

A “promoter” is a nucleic acid control sequence that directs the transcription of a nucleic acid. As used herein, a promoter includes the necessary nucleic acid sequences near the start site of transcription.

The term “protein” refers to molecules that comprise an amino acid sequence, wherein the amino acids are linked by peptide bonds.

“Transformation” refers to the transfer of a nucleic acid into a host organism or into the genome of a host organism, resulting in genetically stable inheritance. Host organisms containing the transformed nucleic acid are referred to as “recombinant,” “transgenic,” or “transformed” organisms. Thus, nucleic acids of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. Typically, expression vectors include, for example, one or more cloned genes under the transcriptional control of 5′ and 3′ regulatory sequences and a selectable marker. Such vectors also can contain a promoter regulatory region (e.g., a regulatory region controlling inducible or constitutive, environmentally- or developmentally-regulated, or location-specific expression), a transcription initiation start site, a ribosome binding site, a transcription termination site, and/or a polyadenylation signal.

The term “transformed cell” refers to a cell that has undergone a transformation. Thus, a transformed cell comprises the parent's genome and an inheritable genetic modification.

The terms “triacylglyceride,” “triacylglycerol,” “triglyceride,” and “TAG” are esters comprised of glycerol and three fatty acids.

### Microbe Engineering

### A. Overview

Genes and gene products may be introduced into microbial host cells. Suitable host cells for expression of the genes and nucleic acid molecules are microbial hosts that can be found broadly within the fungal or bacterial families Examples of suitable host strains include but are not limited to fungal or yeast species, such as Arxula, Aspegillus, Aurantiochytrium, Candida, Claviceps, Cryptococcus, Cunninghamella, Hansenula, Kluyveromyces, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Yarrowia, or bacterial species, such as members of proteobacteria and actinomycetes, as well as the genera Acinetobacter, Arthrobacter, Brevibacterium, Acidovorax, Bacillus, Clostridia, Streptomyces, Escherichia, Salmonella, Pseudomonas, and Cornyebacterium. Yarrowia lipolytica and Arxula adeninivorans are suited for use as a host microorganism because they can accumulate a large percentage of their weight as triacylglycerols.

Microbial expression systems and expression vectors containing regulatory sequences that direct high level expression of foreign proteins are known to those skilled in the art. Any of these could be used to construct chimeric genes to produce any one of the gene products of the instant sequences. These chimeric genes could then be introduced into appropriate microorganisms via transformation techniques to provide high-level expression of the enzymes.

For example, a gene encoding an enzyme can be cloned in a suitable plasmid, and an aforementioned starting parent strain as a host can be transformed with the resulting plasmid. This approach can increase the copy number of each of the genes encoding the enzymes and, as a result, the activities of the enzymes can be increased. The plasmid is not particularly limited so long as it renders a desired genetic modification inheritable to the microorganism's progeny.

Vectors or cassettes useful for the transformation of suitable host cells are well known. Typically the vector or cassette contains sequences that direct the transcription and translation of the relevant gene, a selectable marker, and sequences that allow autonomous replication or chromosomal integration. Suitable vectors comprise a region 5′ of the gene harboring transcriptional initiation controls and a region 3′ of the DNA fragment which controls transcriptional termination. In certain embodiments both control regions are derived from genes homologous to the transformed host cell, although it is to be understood that such control regions need not be derived from the genes native to the specific species chosen as a production host. Promoters, cDNAs, and 3′UTRs, as well as other elements of the vectors, can be generated through cloning techniques using fragments isolated from native sources (see, e.g., Green & Sambrook, Molecular Cloning: A Laboratory Manual, (4th ed., 2012); U.S. Pat. No. 4,683,202 (incorporated by reference)). Alternatively, elements can be generated synthetically using known methods (see, e.g., Gene 164:49-53 (1995)).

### B. Homologous Recombination

Homologous recombination is the ability of complementary DNA sequences to align and exchange regions of homology. Transgenic DNA (“donor”) containing sequences homologous to the genomic sequences being targeted (“template”) is introduced into the organism and then undergoes recombination into the genome at the site of the corresponding homologous genomic sequences.

The ability to carry out homologous recombination in a host organism has many practical implications for what can be carried out at the molecular genetic level and is useful in the generation of a microbe that can produce a desired product. By its nature homologous recombination is a precise gene targeting event and, hence, most transgenic lines generated with the same targeting sequence will be essentially identical in terms of phenotype, necessitating the screening of far fewer transformation events. Homologous recombination also targets gene insertion events into the host chromosome, potentially resulting in excellent genetic stability, even in the absence of genetic selection. Because different chromosomal loci will likely impact gene expression, even from exogenous promoters/UTRs, homologous recombination can be a method of querying loci in an unfamiliar genome environment and to assess the impact of these environments on gene expression.

A particularly useful genetic engineering approach using homologous recombination is to co-opt specific host regulatory elements, such as promoters/UTRs, to drive heterologous gene expression in a highly specific fashion.

Because homologous recombination is a precise gene targeting event, it can be used to precisely modify any nucleotide(s) within a gene or region of interest, so long as sufficient flanking regions have been identified. Therefore, homologous recombination can be used as a means to modify regulatory sequences impacting gene expression of RNA and/or proteins. It can also be used to modify protein coding regions in an effort to modify enzyme activities such as substrate specificity, affinities and Km, thereby affecting a desired change in the metabolism of the host cell. Homologous recombination provides a powerful means to manipulate the host genome resulting in gene targeting, gene conversion, gene deletion, gene duplication, gene inversion, and exchanging gene expression regulatory elements such as promoters, enhancers and 3′UTRs.

Homologous recombination can be achieved by using targeting constructs containing pieces of endogenous sequences to “target” the gene or region of interest within the endogenous host cell genome. Such targeting sequences can either be located 5′ of the gene or region of interest, 3′ of the gene/region of interest or even flank the gene/region of interest. Such targeting constructs can be transformed into the host cell either as a supercoiled plasmid DNA with additional vector backbone, a PCR product with no vector backbone, or as a linearized molecule. In some cases, it may be advantageous to first expose the homologous sequences within the transgenic DNA (donor DNA) by cutting the transgenic DNA with a restriction enzyme. This step can increase the recombination efficiency and decrease the occurrence of undesired events. Other methods of increasing recombination efficiency include using PCR to generate transforming transgenic DNA containing linear ends homologous to the genomic sequences being targeted.

### C. Vectors and Vector Components

Vectors for transforming microorganisms in accordance with the present invention can be prepared by known techniques familiar to those skilled in the art in view of the disclosure herein. A vector typically contains one or more genes, in which each gene codes for the expression of a desired product (the gene product) and is operably linked to one or more control sequences that regulate gene expression or target the gene product to a particular location in the recombinant cell.

### 1. Control Sequences

Control sequences are nucleic acids that regulate the expression of a coding sequence or direct a gene product to a particular location in or outside a cell. Control sequences that regulate expression include, for example, promoters that regulate transcription of a coding sequence and terminators that terminate transcription of a coding sequence. Another control sequence is a 3′ untranslated sequence located at the end of a coding sequence that encodes a polyadenylation signal. Control sequences that direct gene products to particular locations include those that encode signal peptides, which direct the protein to which they are attached to a particular location inside or outside the cell.

Thus, an exemplary vector design for expression of a gene in a microbe contains a coding sequence for a desired gene product (for example, a selectable marker, or an enzyme) in operable linkage with a promoter active in yeast. Alternatively, if the vector does not contain a promoter in operable linkage with the coding sequence of interest, the coding sequence can be transformed into the cells such that it becomes operably linked to an endogenous promoter at the point of vector integration.

The promoter used to express a gene can be the promoter naturally linked to that gene or a different promoter.

A promoter can generally be characterized as constitutive or inducible. Constitutive promoters are generally active or function to drive expression at all times (or at certain times in the cell life cycle) at the same level. Inducible promoters, conversely, are active (or rendered inactive) or are significantly up- or down-regulated only in response to a stimulus. Both types of promoters find application in the methods of the invention. Inducible promoters useful in the invention include those that mediate transcription of an operably linked gene in response to a stimulus, such as an exogenously provided small molecule, temperature (heat or cold), lack of nitrogen in culture media, etc. Suitable promoters can activate transcription of an essentially silent gene or upregulate, e.g., substantially, transcription of an operably linked gene that is transcribed at a low level.

Inclusion of termination region control sequence is optional, and if employed, then the choice is primarily one of convenience, as the termination region is relatively interchangeable. The termination region may be native to the transcriptional initiation region (the promoter), may be native to the DNA sequence of interest, or may be obtainable from another source (See, e.g., Chen & Orozco, Nucleic Acids Research 16:8411 (1988)).

### 2. Genes and Codon Optimization

Typically, a gene includes a promoter, a coding sequence, and termination control sequences. When assembled by recombinant DNA technology, a gene may be termed an expression cassette and may be flanked by restriction sites for convenient insertion into a vector that is used to introduce the recombinant gene into a host cell. The expression cassette can be flanked by DNA sequences from the genome or other nucleic acid target to facilitate stable integration of the expression cassette into the genome by homologous recombination. Alternatively, the vector and its expression cassette may remain unintegrated (e.g., an episome), in which case, the vector typically includes an origin of replication, which is capable of providing for replication of the vector DNA.

A common gene present on a vector is a gene that codes for a protein, the expression of which allows the recombinant cell containing the protein to be differentiated from cells that do not express the protein. Such a gene, and its corresponding gene product, is called a selectable marker or selection marker. Any of a wide variety of selectable markers can be employed in a transgene construct useful for transforming the organisms of the invention.

For optimal expression of a recombinant protein, it is beneficial to employ coding sequences that produce mRNA with codons optimally used by the host cell to be transformed. Thus, proper expression of transgenes can require that the codon usage of the transgene matches the specific codon bias of the organism in which the transgene is being expressed. The precise mechanisms underlying this effect are many, but include the proper balancing of available aminoacylated tRNA pools with proteins being synthesized in the cell, coupled with more efficient translation of the transgenic messenger RNA (mRNA) when this need is met. When codon usage in the transgene is not optimized, available tRNA pools are not sufficient to allow for efficient translation of the transgenic mRNA resulting in ribosomal stalling and termination and possible instability of the transgenic mRNA. Resources for codon-optimization of gene sequences are described in Puigbo et al. (Nucleic Acids Research 35:W126-31 (2007)), and principles underlying codon optimization strategies are described in Angov (Biotechnology Journal 6:650-69 (2011)). Public databases providing statistics for codon usage by different organisms are available, including at www.kazusa.or.jp/codon/ and other publicly available databases and resources.

### D. Transformation

Cells can be transformed by any suitable technique including, e.g., biolistics, electroporation, glass bead transformation, and silicon carbide whisker transformation. Any convenient technique for introducing a transgene into a microorganism can be employed in the present invention. Transformation can be achieved by, for example, the method of D. M. Morrison (Methods in Enzymology 68:326 (1979)), the method by increasing permeability of recipient cells for DNA with calcium chloride (Mandel & Higa, J. Molecular Biology, 53:159 (1970)), or the like.

Examples of expression of transgenes in oleaginous yeast (e.g., Yarrowia lipolytica) can be found in the literature (Bordes et al., J. Microbiological Methods, 70:493 (2007); Chen et al., Applied Microbiology & Biotechnology 48:232 (1997)). Examples of expression of exogenous genes in bacteria such as E. coli are well known (Green & Sambrook, Molecular Cloning: A Laboratory Manual, (4th ed., 2012)).

Vectors for transformation of microorganisms in accordance with the present invention can be prepared by known techniques familiar to those skilled in the art. In one embodiment, an exemplary vector design for expression of a gene in a microorganism contains a gene encoding an enzyme in operable linkage with a promoter active in the microorganism. Alternatively, if the vector does not contain a promoter in operable linkage with the gene of interest, the gene can be transformed into the cells such that it becomes operably linked to a native promoter at the point of vector integration. The vector can also contain a second gene that encodes a protein. Optionally, one or both gene(s) is/are followed by a 3′ untranslated sequence containing a polyadenylation signal. Expression cassettes encoding the two genes can be physically linked in the vector or on separate vectors. Co-transformation of microbes can also be used, in which distinct vector molecules are simultaneously used to transform cells (Protist 155:381-93 (2004)). The transformed cells can be optionally selected based upon the ability to grow in the presence of the antibiotic or other selectable marker under conditions in which cells lacking the resistance cassette would not grow.

### Exemplary Cells, Nucleic Acids, Compositions, and Methods

### A. Transformed Cell

In some embodiments, the transformed cell is a prokaryotic cell, such as a bacterial cell. In some embodiments, the cell is a eukaryotic cell, such as a mammalian cell, a yeast cell, a filamentous fungi cell, a protist cell, an algae cell, an avian cell, a plant cell, or an insect cell. In some embodiments, the cell is a yeast. Those with skill in the art will recognize that many forms of filamentous fungi produce yeast-like growth, and the definition of yeast herein encompasses such cells. The cell may cell may be selected from the group consisting of algae, bacteria, molds, fungi, plants, and yeasts. The cell may be a yeast, fungus, or yeast-like algae. The cell may be selected from thraustochytrids (Aurantiochytrium) and achlorophylic unicellular algae (Prototheca).

The cell may be selected from the group consisting of Arxula, Aspegillus, Aurantiochytrium, Candida, Claviceps, Cryptococcus, Cunninghamella, Geotrichum, Hansenula, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia. It is specifically contemplated that one or more of these cell types may be excluded from embodiments of this invention.

The cell may be selected from the group of consisting of Arxula adeninivorans, Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, Wickerhamomyces ciferrii, and Yarrowia lipolytica. It is specifically contemplated that one or more of these cell types may be excluded from embodiments of this invention.

The cell may be Saccharomyces cerevisiae, Yarrowia lipolytica, or Arxula adeninivorans.

In certain embodiments, the transformed cell comprises at least 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, or more lipid as measured by % dry cell weight, or any range derivable therein. In some embodiments, the transformed cell comprises C18 fatty acids at a concentration of at least 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or higher as a percentage of total C16 and C18 fatty acids in the cell, or any range derivable therein.

In some embodiments, the transformed cell comprises oleic acid at a concentration of at least 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or higher as a percentage of total C16 and C18 fatty acids in the cell, or any range derivable therein. In some embodiments, the transformed cell comprises a linear fatty acid with a chain length of 14-20 carbons with a methyl branch at the 49, MO, or 411 position (e.g., 10-methylstearic acid) at a concentration of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight or higher as a percentage of total fatty acids in the cell, or any range derivable therein. In some embodiments, the fatty acid has a chain length of 14, 15, 16, 17, 18, 19, or 20 carbons, or any range derivable therein.

A cell may be modified to increase its oleate content, which serves as a substrate for 10-methylstearate synthesis. Genetic modifications that increase oleate content are known (see, e.g., PCT Patent Application Publication No. WO16/094520, published Jun. 16, 2016, hereby incorporated by reference in its entirety). For example, a cell may comprise a 412 desaturase knockdown or knockout, which favors the accumulation of oleate and disfavors the production of linoleate. A cell may comprise a recombinant Δ9 desaturase gene, which favors the production of oleate and disfavors the accumulation of stearate. The recombinant Δ9 desaturase gene may be, for example, the Δ9 desaturase gene from Y. lipolytica, Arxula adeninivorans, or Puccinia graminis. A cell may comprise a recombinant elongase 1 gene, which favors the production of oleate and disfavors the accumulation of palmitate and palmitoleate. The recombinant elongase 1 gene may be the elongase 1 gene from Y. lipolytica. A cell may comprise a recombinant elongase 2 gene, which favors the production of oleate and disfavors the accumulation of palmitate and palmitoleate. The recombinant elongase 2 gene may be the elongase 2 gene from R. norvegicus.

A cell may be modified to increase its triacylglycerol content, thereby increasing its 10-methylstearate content. Genetic modifications that increase triacylglycerol content are known (see, e.g., PCT Patent Application Publication No. WO16/094520, published Jun. 16, 2016, hereby incorporated by reference in its entirety). A cell may comprise a recombinant diacylglycerol acyltransferase gene (e.g., DGAT1, DGAT2, or DGAT3), which favors the production of triacylglycerols and disfavors the accumulation of diacylglycerols. The recombinant diacylglycerol acyltransferase gene may be, for example, DGAT2 (encoding protein DGA1) from Y. lipolytica, DGAT1 (encoding protein DGA2) from C. purpurea, or DGAT2 (encoding protein DGA1) from R. toruloides. The cell may comprise a glycerol-3-phosphate acyltransferase gene (Sctl) knockdown or knockout, which may favor the accumulation of triacylglycerols, depending on the cell type. The cell may comprise a recombinant glycerol-3-phosphate acyltransferase gene (Sctl) such as the Sctl gene from A. adeninivorans, which may favor the accumulation of triacylglycerols. The cell may comprise a triacylglycerol lipase gene (TGL) knockdown or knockout, which may favor the accumulation of triacylglycerols in the cell.

Various aspects of the invention relate to a transformed cell. The transformed cell may comprise a recombinant methyltransferase gene (e.g., a tmsB gene), a recombinant reductase gene (e.g., a tmsA gene), an exomethylene-substituted lipid, and/or a branched (methyl)lipid. A transformed cell may comprise a tmsC gene. A branched (methyl)lipid may be a carboxylic acid (e.g., 10-methylstearic acid, 10-methylpalmitic acid, 12-methyloleic acid, 13-methyloleic acid, 10-methyl-octadec-12-enoic acid), carboxylate (e.g., 10-methylstearate, 10-methylpalmitate, 12-methyloleate, 13-methyloleate, 10-methyl-octadec-12-enoate), ester (e.g., diacylglycerol, triacylglycerol, phospholipid), thioester (e.g., 10-methylstearyl CoA, 10-methylpalmityl CoA, 12-methyloleoyl CoA, 13-methyloleoyl CoA, 10-methyl-octadec-12-enoyl CoA), or amide. An exomethylene-substituted lipid may be a carboxylic acid (e.g., 10-methylenestearic acid, 10-methylenepalmitic acid, 12-methyleneoleic acid, 13-methyleneoleic acid, 10-methylene-octadec-12-enoic acid), carboxylate (e.g., 10-methylenestearate, 10-methylenepalmitate, 12-methyleneoleate, 13-methyleneoleate, 10-methylene-octadec-12-enoate), ester (e.g., diacylglycerol, triacylglycerol, phospholipid), thioester (e.g., 10-methylenestearyl CoA, 10-methylenepalmityl CoA, 12-methyleneoleoyl CoA, 13-methyleneoleoyl CoA, 10-methylene-octadec-12-enoyl CoA), or amide. It is specifically contemplated that one or more of the above lipids may be excluded from embodiments of this invention.

“Fatty acids” generally exist in a cell as a phospholipid or triacylglycerol, although they may also exist as a monoacylglycerol or diacylglycerol, for example, as a metabolic intermediate. Free fatty acids also exist in the cell in equilibrium between a relatively abundant carboxylate anion and a relatively scarce, neutrally-charged acid. A fatty acid may exist in a cell as a thioester, especially as a thioester with coenzyme A (CoA), during biosynthesis or oxidation. A fatty acid may exist in a cell as an amide, for example, when covalently bound to a protein to anchor the protein to a membrane.

A cell may comprise any one of the nucleic acids described herein, infra (see, e.g., Section B, below).

A branched (methyl)lipid may comprise a saturated branched aliphatic chain (e.g., 10-methylstearic acid, 10-methylpalmitic acid) or an unsaturated branched aliphatic chain (e.g., 12-methyloleic acid, 13-methyloleic acid, 10-methyl-octadec-12-enoic acid). The branched (methyl)lipid may comprise a saturated or unsaturated branched aliphatic chain comprising a branching methyl group.

An exomethylene-substituted lipid may comprise a branched aliphatic chain (e.g., 10-methylenestearic acid, 10-methylenepalmitic acid, 12-methyleneoleic acid, 13-methyleneoleic acid, 10-methylene-octadec-12-enoic acid). The aliphatic chain may be branched because the aliphatic chain is substituted with an exomethylene group.

A branched (methyl)lipid may be 10-methylstearate, or an acid (10-methylstearic acid), ester (e.g., diacylglycerol, triacylglycerol, phospholipid), thioester (e.g., 10-methylstearyl CoA), or amide (e.g., 10-methylstearyl amide) thereof. For example, the branched (methyl)lipid may be a diacylglycerol, triacylglycerol, or phospholipid, and the diacylglycerol, triacylglycerol, or phospholipid may comprise an ester of 10-methylstearate.

An exomethylene-substituted lipid may be 10-methylenestearate, or an acid (10-methylenestearic acid), ester (e.g., diacylglycerol, triacylglycerol, phospholipid), thioester (e.g., 10-methylenestearyl CoA), or amide (e.g., 10-methylenestearyl amide) thereof. For example, the exomethylene-substituted lipid may be a diacylglycerol, triacylglycerol, or phospholipid, and the diacylglycerol, triacylglycerol, or phospholipid may comprise an ester of 10-methylenestearate.

In some embodiments, about, at most about, or at least about 1% of the fatty acids of the cell may be 10-methylstearic acid as measured by % dry cell weight. About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the fatty acids of the cell may be 10-methylstearic acid as measured by % dry cell weight, or any range derivable therein.

In some embodiments, about, at least about, or at most about 1% of the fatty acids of the cell may be 10-methylenestearic acid as measured by % dry cell weight. About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the fatty acids of the cell may be 10-methylenestearic acid as measured by % dry cell weight, or any range derivable therein.

In some embodiments, about, at least about, or at most about 1% by weight of the fatty acids of the cell may be one or more of the branched (methyl)lipids described herein. About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of the fatty acids of the cell may be one or more of the branched (methyl)lipids described herein, or any range derivable therein.

In some embodiments, about, at least about, or at most about 1% by weight of the fatty acids of the cell may one or more of the branched (methyl)lipids described herein (e.g., a linear fatty acid with a chain length of 14-20 carbons with a methyl branch at the 49, MO, or 411 position). About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the fatty acids of the cell may one or more of the branched (methyl)lipids described herein (e.g., a linear fatty acid with a chain length of 14-20 carbons with a methyl branch at the Δ9, Δ10, or Δ11 position), or any range derivable therein.

In some embodiments, the cell may comprise about, at least about, or at most about 1% 10-methylstearic acid as measured by % dry cell weight. The cell may comprise about, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% 10-methylstearic acid as measured by % dry cell weight, or any range derivable therein.

In some embodiments, the cell may comprise about, at least about, or at most about 1% 10-methylenestearic acid as measured by % dry cell weight. The cell may comprise about, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% 10-methylenestearic acid as measured by % dry cell weight, or any range derivable therein.

An unmodified cell of the same type (e.g., species) as a cell of the invention may not comprise 10-methylstearate, or an acid (10-methylstearic acid), ester (e.g., diacylglycerol, triacylglycerol, phospholipid), thioester (e.g., 10-methylstearyl CoA), or amide (e.g., 10-methylstearyl amide) thereof (e.g., wherein the unmodified cell does not comprise a recombinant methyltransferase gene or a recombinant reductase gene). An unmodified cell of the same type (e.g., species) as a cell of the invention may not comprise 10-methylenestearate, or an acid (10-methylenestearic acid), ester (e.g., diacylglycerol, triacylglycerol, phospholipid), thioester (e.g., 10-methylenestearyl CoA), or amide (e.g., 10-methylenestearyl amide) thereof (e.g., wherein the unmodified cell does not comprise a recombinant methyltransferase gene or a recombinant reductase gene). In some embodiments, an unmodified cell of the same species as the cell does not comprise a branched (methyl)lipid and/or an exomethylene-substituted lipid. In some embodiments, an unmodified cell of the same species as the cell does not comprise one or more of the branched (methyl)lipids or exomethylene-substituted lipids described herein.

A cell may constitutively express the protein encoded by a recombinant methyltransferase gene. A cell may constitutively express the protein encoded by a recombinant reductase gene. A cell may constitutively express the protein encoded by a recombinant tmsC gene. A cell may constitutively express a methyltransferase protein. A cell may constitutively express a reductase protein. A cell may constitutively express a tmsC protein.

### B. Nucleic Acids

Various aspects of the invention relate to a nucleic acid comprising a recombinant methyltransferase gene, a recombinant reductase gene, or both. The nucleic acid may be, for example, a plasmid. In some embodiments, a recombinant methyltransferase gene and/or a recombinant reductase gene is integrated into the genome of a cell, and thus, the nucleic acid may be a chromosome. In some embodiments, the invention relates to a cell comprising a recombinant methyltransferase gene, e.g., wherein the recombinant methyltransferase gene is present in a plasmid or chromosome. In some embodiments, the invention relates to a cell comprising a recombinant reductase gene, e.g., wherein the recombinant reductase gene is present in a plasmid or chromosome. A recombinant methyltransferase gene and a recombinant reductase gene may be present in a cell in the same nucleic acid (e.g., same plasmid or chromosome) or in different nucleic acids (e.g., different plasmids or chromosomes).

A nucleic acid may be inheritable to the progeny of a transformed cell. A gene such as a recombinant methyltransferase gene or recombinant reductase gene may be inheritable because it resides on a plasmid or chromosome. In certain embodiments, a gene may be inheritable because it is integrated into the genome of the transformed cell.

A gene may comprise conservative substitutions, deletions, and/or insertions while still encoding a protein that has activity. For example, codons may be optimized for a particular host cell, different codons may be substituted for convenience, such as to introduce a restriction site or to create optimal PCR primers, or codons may be substituted for another purpose. Similarly, the nucleotide sequence may be altered to create conservative amino acid substitutions, deletions, and/or insertions.

Proteins may comprise conservative substitutions, deletions, and/or insertions while still maintaining activity. Conservative substitution tables are well known in the art (Creighton, Proteins (2d. ed., 1992)).

Amino acid substitutions, deletions and/or insertions may readily be made using recombinant DNA manipulation techniques. Methods for the manipulation of DNA sequences to produce substitution, insertion or deletion variants of a protein are well known in the art. These methods include M13 mutagenesis, T7-Gen in vitro mutagenesis (USB, Cleveland, OH), Quick Change Site Directed mutagenesis (Stratagene, San Diego, CA), PCR-mediated site-directed mutagenesis, and other site-directed mutagenesis protocols.

To determine the percent identity of two amino acid sequences or two nucleic acid sequences, the sequences can be aligned for optimal comparison purposes (e.g., gaps can be introduced in one or both of a first and a second amino acid or nucleic acid sequence for optimal alignment and non-identical sequences can be disregarded for comparison purposes). The length of a reference sequence aligned for comparison purposes can be at least 95% of the length of the reference sequence. The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. Unless otherwise specified, when percent identity between two amino acid sequences is referred to herein, it refers to the percent identity as determined using the Needleman and Wunsch (J. Molecular Biology 48:444-453 (1970)) algorithm which has been incorporated into the GAP program in the GCG software package (available at http://www.gcg.com), using a Blosum 62 matrix, a gap weight of 10, and a length weight of 4. In some embodiments, the percent identity between two amino acid sequences is determined the Needleman and Wunsch algorithm using a Blosum 62 matrix or a PAM250 matrix, and a gap weight of 16, 14, 12, 10, 8, 6, or 4 and a length weight of 1, 2, 3, 4, 5, or 6. Unless otherwise specified, when percent identity between two nucleotide sequences is referred to herein, it refers to percent identity as determined using the GAP program in the GCG software package (available at http://www.gcg.com), using a NWSgapdna.CMP matrix and a gap weight of 60 and a length weight of 4. In yet another embodiment, the percent identity between two nucleotide sequences can be determined using a gap weight of 40, 50, 60, 70, or 80 and a length weight of 1, 2, 3, 4, 5, or 6. In another embodiment, the percent identity between two amino acid or nucleotide sequences can be determined using the algorithm of E. Meyers and W. Miller (Computer Applications in the Biosciences 4:11-17 (1988)) which has been incorporated into the ALIGN program (version 2.0 or 2.0U), using a PAM120 weight residue table, a gap length penalty of 12 and a gap penalty of 4.

Exemplary computer programs which can be used to determine identity between two sequences include, but are not limited to, the suite of BLAST programs, e.g., BLASTN, MEGABLAST, BLASTX, TBLASTN, TBLASTX, and BLASTP, and Clustal programs, e.g., ClustalW, ClustalX, and Clustal Omega.

Sequence searches are typically carried out using the BLASTN program, when evaluating a given nucleic acid sequence relative to nucleic acid sequences in the GenBank DNA Sequences and other public databases. The BLASTX program is effective for searching nucleic acid sequences that have been translated in all reading frames against amino acid sequences in the GenBank Protein Sequences and other public databases.

An alignment of selected sequences in order to determine “% identity” between two or more sequences is performed using for example, the CLUSTAL-W program.

A “coding sequence” or “coding region” refers to a nucleic acid molecule having sequence information necessary to produce a protein product, such as an amino acid or polypeptide, when the sequence is expressed. The coding sequence may comprise and/or consist of untranslated sequences (including introns or 5′ or 3′ untranslated regions) within translated regions, or may lack such intervening untranslated sequences (e.g., as in cDNA).

The abbreviation used throughout the specification to refer to nucleic acids comprising and/or consisting of nucleotide sequences are the conventional one-letter abbreviations. Thus when included in a nucleic acid, the naturally occurring encoding nucleotides are abbreviated as follows: adenine (A), guanine (G), cytosine (C), thymine (T) and uracil (U). Also, unless otherwise specified, the nucleic acid sequences presented herein is the 5′→3′ direction.

As used herein, the term “complementary” and derivatives thereof are used in reference to pairing of nucleic acids by the well-known rules that A pairs with T or U and C pairs with G. Complement can be “partial” or “complete”. In partial complement, only some of the nucleic acid bases are matched according to the base pairing rules; while in complete or total complement, all the bases are matched according to the pairing rule. The degree of complement between the nucleic acid strands may have significant effects on the efficiency and strength of hybridization between nucleic acid strands as well known in the art. The efficiency and strength of said hybridization depends upon the detection method.

Any nucleic acid that is referred to herein as having a certain percent sequence identity to a sequence set forth in a SEQ ID NO, includes nucleic acids that have the certain percent sequence identity to the complement of the sequence set forth in the SEQ ID NO.

i. Nucleic Acids Comprising a Recombinant Methyltransferase Gene

A methyltransferase gene (e.g., a recombinant methyltransferase gene) encodes a methyltransferase protein, which is an enzyme capable of transferring a carbon atom and one or more protons bound thereto from a substrate such as S-adenosyl methionine to a fatty acid such as oleic acid (e.g., wherein the fatty acid is present as a free fatty acid, carboxylate, phospholipid, diacylglycerol, or triacylglycerol). A methyltransferase gene (e.g., a recombinant methyltransferase gene) may comprise any one of the nucleotide sequences set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, and SEQ ID NO:81. A methyltransferase gene (e.g., a recombinant methyltransferase gene) may be a 10-methylstearic B gene (tmsB) as described herein, or a biologically-active portion thereof (i.e., wherein the biologically-active portion thereof comprises methyltransferase activity).

A methyltransferase gene (e.g., a recombinant methyltransferase gene) may be derived from a gram-positive species of Actinobacteria, such as Mycobacteria, Corynebacteria, Nocardia, Streptomyces, or Rhodococcus. A methyltransferase gene (e.g., a recombinant methyltransferase gene) may be selected from the group consisting of Mycobacterium smegmatis gene tmsB, Agromyces subbeticus gene tmsB, Amycolicicoccus subflavus gene tmsB, Corynebacterium glutamicum gene tmsB, Corynebacterium glyciniphilium gene tmsB, Knoella aerolata gene tmsB, Mycobacterium austroafricanum gene tmsB, Mycobacterium gilvum gene tmsB, Mycobacterium indicus pranii gene tmsB, Mycobacterium phlei gene tmsB, Mycobacterium tuberculosis gene tmsB, Mycobacterium vanbaalenii gene tmsB, Rhodococcus opacus gene tmsB, Streptomyces regnsis gene tmsB, Thermobifida fusca gene tmsB, and Thermomonospora curvata gene tmsB. It is specifically contemplated that one or more of the above methyltransferase genes may be excluded from embodiments of this invention.

A recombinant methyltransferase gene may be recombinant because it is operably-linked to a promoter other than the naturally-occurring promoter of the methyltransferase gene. Such genes may be useful to drive transcription in a particular species of cell. A recombinant methyltransferase gene may be recombinant because it contains one or more nucleotide substitutions relative to a naturally-occurring methyltransferase gene. Such genes may be useful to increase the translation efficiency of the methyltransferase gene's mRNA transcript in a particular species of cell.

A nucleic acid may comprise a recombinant methyltransferase gene and a promoter, wherein the recombinant methyltransferase gene and promoter are operably-linked. The recombinant methyltransferase gene and promoter may be derived from different species. For example, the recombinant methyltransferase gene may encode the methyltransferase protein of a gram-positive species of Actinobacteria, and the recombinant methyltransferase gene may be operably-linked to a promoter that can drive transcription in another phylum of bacteria (e.g., a Proteobacterium, such as E. coli) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise a recombinant methyltransferase gene, and the recombinant methyltransferase gene may be operably-linked to a promoter capable of driving transcription of the recombinant methyltransferase gene in the cell. The cell may be a species of yeast, and the promoter may be a yeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant, and the promoter may be a plant promoter.

A recombinant methyltransferase gene may be operably-linked to a promoter that cannot drive transcription in the cell from which the recombinant methyltransferase gene originated. For example, the promoter may not be capable of binding an RNA polymerase of the cell from which a recombinant methyltransferase gene originated. In some embodiments, the promoter cannot bind a prokaryotic RNA polymerase and/or initiate transcription mediated by a prokaryotic RNA polymerase. In some embodiments, a recombinant methyltransferase gene is operably-linked to a promoter that cannot drive transcription in the cell from which the protein encoded by the gene originated. For example, the promoter may not be capable of binding an RNA polymerase of a cell that naturally expresses the methyltransferase enzyme encoded by a recombinant methyltransferase gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A recombinant methyltransferase gene may comprise a nucleotide sequence with at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, or SEQ ID NO:81. A recombinant methyltransferase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity (or any range derivable therein) with 150, 200, 250, 300, 350, 400, 450, 500, 550, 600, 650, 700, 750, 800, 850, 900, 950, 1000, 1050, 1100, 1150, 1200, 1250, or 1300 contiguous base pairs (or any range derivable therein) starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, or SEQ ID NO:81. A recombinant methyltransferase may or may not have 100% sequence identity with any one of the nucleotide sequences set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, or SEQ ID NO:81. A recombinant methyltransferase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, or SEQ ID NO:81. A recombinant methyltransferase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, SEQ ID NO:75, or SEQ ID NO:81, and the recombinant methyltransferase gene may encode a methyltransferase protein with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76. For example, SEQ ID NO:81 is a gene that is codon-optimized for expression in yeast. SEQ ID NO:81 has about 70% sequence identity (69.86% sequence identity) with SEQ ID NO:3, and the protein encoded by SEQ ID NO:81 has 100% sequence identity with the amino acid sequence set forth in by SEQ ID NO:4. Thus, even though SEQ ID NO:81 and SEQ ID NO:3 have 69.86% sequence identity, the two nucleotide sequences encode the same amino acid sequence.

A recombinant methyltransferase gene may vary from a naturally-occurring methyltransferase gene because the recombinant methyltransferase gene may be codon-optimized for expression in a eukaryotic cell, such as a plant cell, algae cell, or yeast cell. A cell may comprise a recombinant methyltransferase gene, wherein the recombinant methyltransferase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant methyltransferase gene may vary from a naturally-occurring methyltransferase gene or may be unchanged from a naturally-occurring methyltransferase gene. For example, a recombinant methyltransferase gene may comprise a nucleotide sequence with at least about 65% sequence identity with the naturally-occurring nucleotide sequence set forth in SEQ ID NO:3, SEQ ID NO:7, SEQ ID NO:11, SEQ ID NO:15, SEQ ID NO:19, SEQ ID NO:25, SEQ ID NO:29, SEQ ID NO:35, SEQ ID NO:41, SEQ ID NO:45, SEQ ID NO:49, SEQ ID NO:53, SEQ ID NO:59, SEQ ID NO:63, SEQ ID NO:69, or SEQ ID NO:75 (e.g., at least 70%, 75%, 80%, 85%, 90%, 95%, 96%, 97%, 98%, or 99% sequence identity), and at least 5 codons of the nucleotide sequence of the recombinant methyltransferase gene may vary from the naturally-occurring nucleotide sequence (e.g., at least 10, 15, 20, 25, 30, 35, 40, 45, 50, 60, 70, 80, 90, or 100 codons (or any range deriable therein)).

A methyltransferase gene encodes a methyltransferase protein. A methyltransferase protein may be a protein expressed by a gram-positive species of Actinobacteria, such as Mycobacteria, Corynebacteria, Nocardia, Streptomyces, or Rhodococcus. A recombinant methyltransferase gene may encode a naturally-occurring methyltransferase protein even if the recombinant methyltransferase gene is not a naturally-occurring methyltransferase gene. For example, a recombinant methyltransferase gene may vary from a naturally-occurring methyltransferase gene because the recombinant methyltransferase gene is codon-optimized for expression in a specific cell. The codon-optimized, recombinant methyltransferase gene and the naturally-occurring methyltransferase gene may nevertheless encode the same naturally-occurring methyltransferase protein.

A recombinant methyltransferase gene may encode a methyltransferase protein selected from Mycobacterium smegmatis enzyme tmsB, Agromyces subbeticus enzyme tmsB, Amycolicicoccus subflavus enzyme tmsB, Corynebacterium glutamicum enzyme tmsB, Corynebacterium glyciniphilium enzyme tmsB, Knoella aerolata enzyme tmsB, Mycobacterium austroafricanum enzyme tmsB, Mycobacterium gilvum enzyme tmsB, Mycobacterium indicus pranii enzyme tmsB, Mycobacterium phlei enzyme tmsB, Mycobacterium tuberculosis enzyme tmsB, Mycobacterium vanbaalenii enzyme tmsB, Rhodococcus opacus enzyme tmsB, Streptomyces regnsis enzyme tmsB, Thermobifida fusca enzyme tmsB, and Thermomonospora curvata enzyme tmsB. It is specifically contemplated that one or more of the above methyltransferase proteins may be excluded from embodiments of this invention. A recombinant methyltransferase gene may encode a methyltransferase protein, and the methyltransferase protein may be substantially identical to any one of the foregoing enzymes, but the recombinant methyltransferase gene may vary from the naturally-occurring gene that encodes the enzyme. The recombinant methyltransferase gene may vary from the naturally-occurring gene because the recombinant methyltransferase gene may be codon-optimized for expression in a specific phylum, class, order, family, genus, species, or strain of cell.

The sequences of naturally-occurring methyltransferase proteins are set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76. A recombinant methyltransferase gene may or may not encode a protein comprising 100% sequence identity with the amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76. For example, a recombinant methyltransferase gene may encode a protein having 100% sequence identity with a biologically-active portion of an amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76.

A recombinant methyltransferase gene may encode a methyltransferase protein having, having at least, or having at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity (or any range derivable therein) with the amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76, or a biologically-active portion thereof. A recombinant methyltransferase gene may encode a methyltransferase protein having at least about 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, or 400% methyltransferase activity (or any range deriable therein) relative to a protein comprising the amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76. A recombinant methyltransferase gene may encode a protein having at least 70%, 75%, 80%, 85%, 90%, 91%, 92%, 93%, 94%, 95%, 96%, 97%, 98%, 99%, 99.9%, or 100% sequence identity with 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous amino acids starting at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 of SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76.

Substrates for the methyltransferase protein may include any fatty acid from 14 to 20 carbons long with an unsaturated double bond in the Δ9, Δ10, or Δ11 position. The methyltransferase protein may be capable of catalyzing the formation of a methylene substitution at the Δ9, Δ10, or Δ11 position of such a substrate.

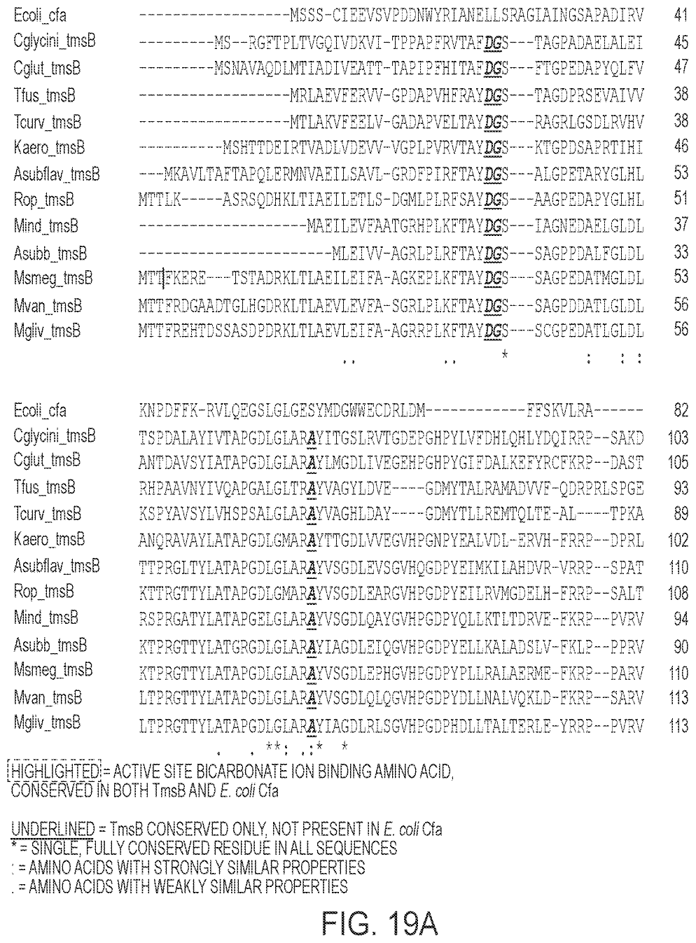

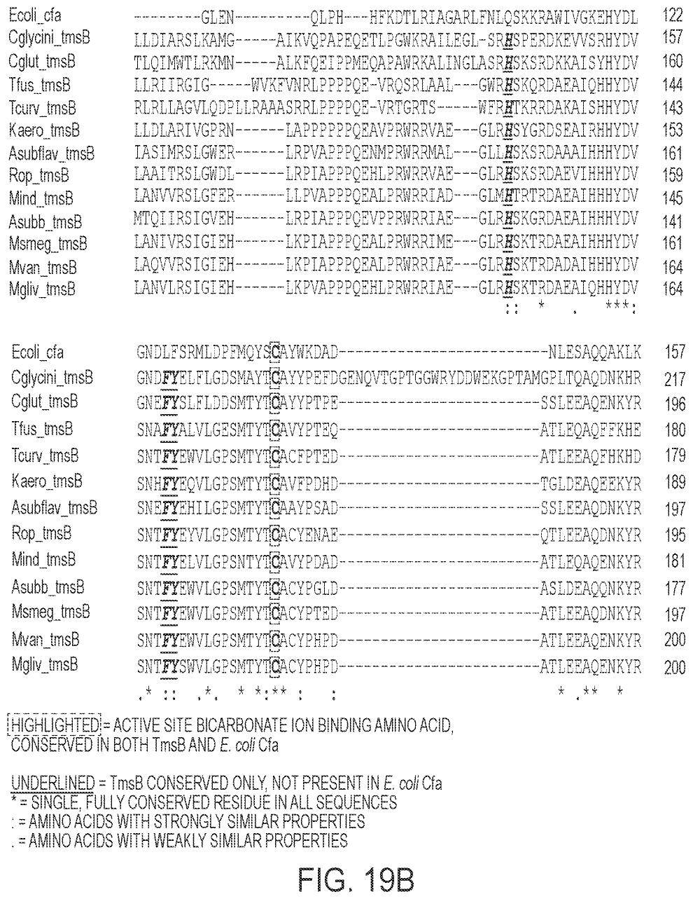

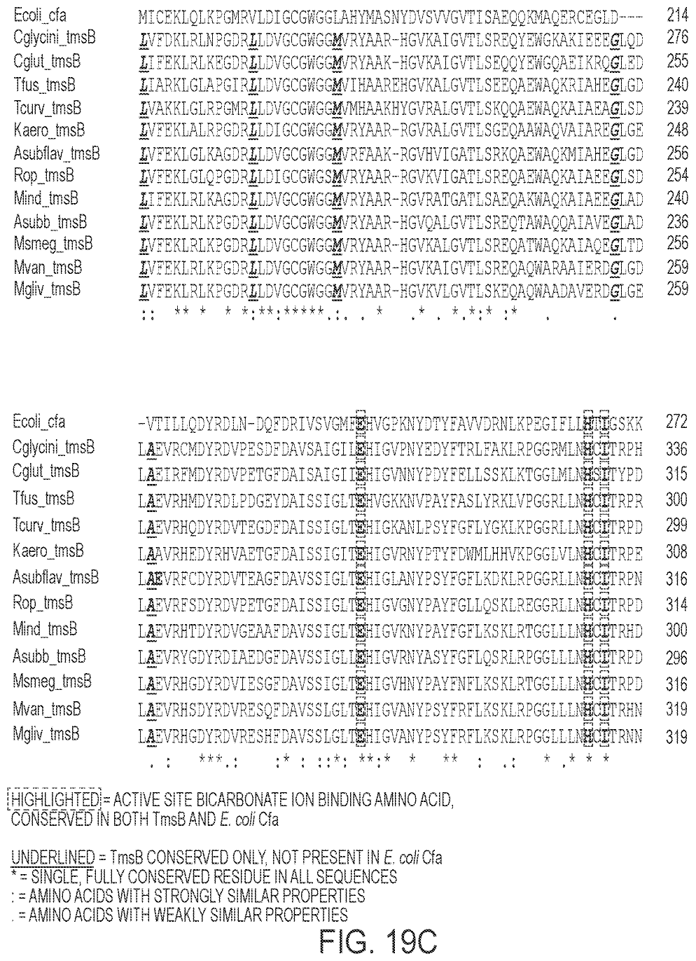

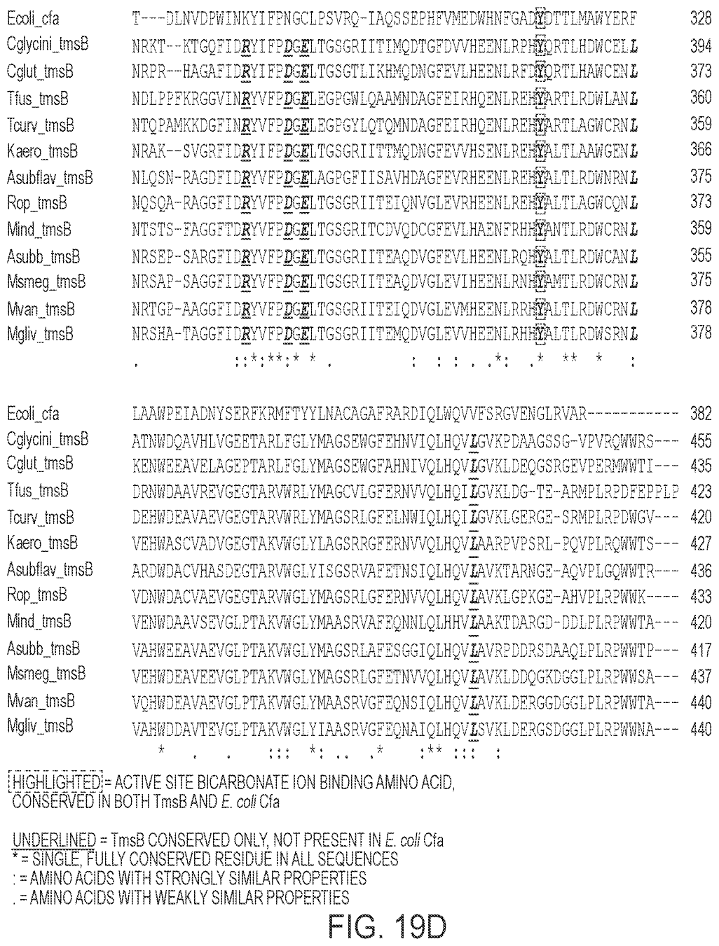

In some embodiments, the recombinant methyltransferase gene encodes a methyltransferase protein that includes an S-adenosylmethionine-dependent methyltransferase domain. In some embodiments the S-adenosylmethionine-dependent methyltransferase domain has, has at least, or has at most 70%, 75%, 80%, 85%, 90%, 91%, 92%, 93%, 94%, 95%, 96%, 97%, 98%, 99%, 99.9%, or 100% sequence identity to amino acids 192-291 of T. curvata TmsB (SEQ ID NO:76) or to a corresponding portion of TmsB from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, or Thermobifida fusca, according to the alignment set forth in FIGS. 19A-D.

In some embodiments, the recombinant methyltransferase gene encodes a methyltransferase protein that has specific amino acids unchanged from the amino acid sequence set forth in SEQ ID NO:4, SEQ ID NO:8, SEQ ID NO:12, SEQ ID NO:16, SEQ ID NO:20, SEQ ID NO:26, SEQ ID NO:30, SEQ ID NO:36, SEQ ID NO:42, SEQ ID NO:46, SEQ ID NO:50, SEQ ID NO:54, SEQ ID NO:60, SEQ ID NO:64, SEQ ID NO:70, or SEQ ID NO:76. The unchanged amino acids can include 1, 2, 3, 4, 5, 6, 7, 8, 9, 10, 11, 12, 13, 14, 15, 16, 17, 18, 19, 20, 21, 22, 23, 24, 25, 26, 27, 28, or 29 amino acids selected from D23, G24, A59, H128, F147, Y148, L180, L193, M203, G236, A241, R313, R318, E320, L359, L400, V196, G197, C198, G199, W200, G201, G202, T219, L220, Q246, D247, Y248, and D262 of T. curvata TmsB (SEQ ID NO:76) or corresponding amino acids in TmsB from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, or Thermobifida fusca, according to the alignment set forth in FIGS. 19A-D.

ii. Nucleic Acids Comprising a Recombinant Reductase Gene

A reductase gene (e.g., a recombinant reductase gene) encodes a reductase protein, which is an enzyme capable of reducing, often in an NADPH-dependent manner, a double bond of a fatty acid (e.g., wherein the fatty acid is present as a free fatty acid, carboxylate, phospholipid, diacylglycerol, or triacylglycerol). A reductase gene (e.g., a recombinant reductase gene) may comprise any one of the nucleotide sequences set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, and SEQ ID NO:80. A reductase gene (e.g., a recombinant reductase gene) may be a 10-methylstearic A gene (tmsA) as described herein, or a biologically-active portion thereof (i.e., wherein the biologically-active portion thereof comprises reductase activity).

A reductase gene (e.g., a recombinant reductase gene) may be derived from a gram-positive species of Actinobacteria, such as Mycobacteria, Corynebacteria, Nocardia, Streptomyces, or Rhodococcus. A reductase gene (e.g., a recombinant reductase gene) may be selected from the group consisting of Mycobacterium smegmatis gene tmsA, Agromyces subbeticus gene tmsA, Amycolicicoccus subflavus gene tmsA, Corynebacterium glutamicum gene tmsA, Corynebacterium glyciniphilium gene tmsA, Knoella aerolata gene tmsA, Mycobacterium austroafricanum gene tmsA, Mycobacterium gilvum gene tmsA, Mycobacterium indicus pranii gene tmsA, Mycobacterium phlei gene tmsA, Mycobacterium tuberculosis gene tmsA, Mycobacterium vanbaalenii gene tmsA, Rhodococcus opacus gene tmsA, Streptomyces regnsis gene tmsA, Thennobifida fusca gene tmsA, and Thermomonospora curvata gene tmsA. It is specifically contemplated that one or more of the above reductase genes may be excluded from embodiments of this invention.

A recombinant reductase gene may be recombinant because it is operably-linked to a promoter other than the naturally-occurring promoter of the reductase gene. Such genes may be useful to drive transcription in a particular species of cell. A recombinant reductase gene may be recombinant because it contains one or more nucleotide substitutions relative to a naturally-occurring reductase gene. Such genes may be useful to increase the translation efficiency of the reductase gene's mRNA transcript in a particular species of cell.

A nucleic acid may comprise a recombinant reductase gene and a promoter, wherein the recombinant reductase gene and promoter are operably-linked. The recombinant reductase gene and promoter may be derived from different species. For example, the recombinant reductase gene may encode the reductase protein of a gram-positive species of Actinobacteria, and the recombinant reductase gene may be operably-linked to a promoter that can drive transcription in another phylum of bacteria (e.g., a Proteobacterium, such as E. coli) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise a recombinant reductase gene, and the recombinant reductase gene may be operably-linked to a promoter capable of driving transcription of the recombinant reductase gene in the cell. The cell may be a species of yeast, and the promoter may be a yeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant, and the promoter may be a plant promoter.

A recombinant reductase gene may be operably-linked to a promoter that cannot drive transcription in the cell from which the recombinant reductase gene originated. For example, the promoter may not be capable of binding an RNA polymerase of the cell from which a recombinant reductase gene originated. In some embodiments, the promoter cannot bind a prokaryotic RNA polymerase and/or initiate transcription mediated by a prokaryotic RNA polymerase. In some embodiments, a recombinant reductase gene is operably-linked to a promoter that cannot drive transcription in the cell from which the protein encoded by the gene originated. For example, the promoter may not be capable of binding an RNA polymerase of a cell that naturally expresses the reductase enzyme encoded by a recombinant reductase gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A recombinant reductase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, or SEQ ID NO:80. A recombinant reductase gene may comprise a nucleotide sequence with, with at least, with at most 65%, 70%, 75%, 80%, 85%, 90%, 95%, 96%, 97%, 98%, or 99% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, or SEQ ID NO:80. A recombinant reductase may or may not have 100% sequence identity with any one of the nucleotide sequences set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, or SEQ ID NO:80. A recombinant reductase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, or SEQ ID NO:80. A recombinant reductase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, SEQ ID NO:73, or SEQ ID NO:80, and the recombinant reductase gene may encode a reductase protein with at least about 95%, 96%, 97%, 98%, 99%, or 100% sequence identity with the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74. For example, SEQ ID NO:80 is a gene that is codon-optimized for expression in yeast. SEQ ID NO:80 has about 70% sequence identity (70.09% sequence identity) with SEQ ID NO:1, and the protein encoded by SEQ ID NO:80 has at least about 99% sequence identity with the amino acid sequence set forth in SEQ ID NO:2. The protein encoded by SEQ ID NO:1 has 100% sequence identity with the amino acid sequence set forth in SEQ ID NO:2.

A recombinant reductase gene may vary from a naturally-occurring reductase gene because the recombinant reductase gene may be codon-optimized for expression in a eukaryotic cell, such as a plant cell, algae cell, or yeast cell. A cell may comprise a recombinant reductase gene, wherein the recombinant reductase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant reductase gene may vary from a naturally-occurring reductase gene or may be unchanged from a naturally-occurring reductase gene. For example, a recombinant reductase gene may comprise a nucleotide sequence with at least 65% sequence identity with the naturally-occurring nucleotide sequence set forth in SEQ ID NO:1, SEQ ID NO:5, SEQ ID NO:9, SEQ ID NO:13, SEQ ID NO:17, SEQ ID NO:23, SEQ ID NO:27, SEQ ID NO:33, SEQ ID NO:39, SEQ ID NO:43, SEQ ID NO:47, SEQ ID NO:51, SEQ ID NO:57, SEQ ID NO:61, SEQ ID NO:67, or SEQ ID NO:73 (e.g., at least 70%, 75%, 80%, 85%, 90%, 95%, 96%, 97%, 98%, or 99% sequence identity), and at least 5 codons of the nucleotide sequence of the recombinant reductase gene may vary from the naturally-occurring nucleotide sequence (e.g., at least 10, 15, 20, 25, 30, 35, 40, 45, 50, 60, 70, 80, 90, or 100 codons).

A reductase gene encodes a reductase protein. A reductase protein may be a protein expressed by a gram-positive species of Actinobacteria, such as Mycobacteria, Corynebacteria, Nocardia, Streptomyces, or Rhodococcus. A recombinant reductase gene may encode a naturally-occurring reductase protein even if the recombinant reductase gene is not a naturally-occurring reductase gene. For example, a recombinant reductase gene may vary from a naturally-occurring reductase gene because the recombinant reductase gene is codon-optimized for expression in a specific cell. The codon-optimized, recombinant reductase gene and the naturally-occurring reductase gene may nevertheless encode the same naturally-occurring reductase protein.

A recombinant reductase gene may encode a reductase protein selected from Mycobacterium smegmatis enzyme tmsA, Agromyces subbeticus enzyme tmsA, Amycolicicoccus subflavus enzyme tmsA, Corynebacterium glutamicum enzyme tmsA, Corynebacterium glyciniphilium enzyme tmsA, Knoella aerolata enzyme tmsA, Mycobacterium austroafricanum enzyme tmsA, Mycobacterium gilvum enzyme tmsA, Mycobacterium indicus pranii enzyme tmsA, Mycobacterium phlei enzyme tmsA, Mycobacterium tuberculosis enzyme tmsA, Mycobacterium vanbaalenii enzyme tmsA, Rhodococcus opacus enzyme tmsA, Streptomyces regnsis enzyme tmsA, Thennoblfida fusca enzyme tmsA, and Thermomonospora curvata enzyme tmsA. It is specifically contemplated that one or more of the above reductase proteins may be excluded from embodiments of this invention. A recombinant reductase gene may encode a reductase protein, and the reductase protein may be substantially identical to any one of the foregoing enzymes, but the recombinant reductase gene may vary from the naturally-occurring gene that encodes the enzyme. The recombinant reductase gene may vary from the naturally-occurring gene because the recombinant reductase gene may be codon-optimized for expression in a specific phylum, class, order, family, genus, species, or strain of cell.

The sequences of naturally-occurring reductase proteins are set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74. A recombinant reductase gene may or may not encode a protein comprising 100% sequence identity with the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74. For example, a recombinant reductase gene may encode a protein having 100% sequence identity with a biologically-active portion of an amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74.

A recombinant reductase gene may encode a reductase protein having, having at least, or having at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74, or a biologically-active portion thereof. A recombinant reductase gene may encode a reductase protein having about, at least about, or at most about 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, or 400% reductase activity relative to a protein comprising the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74. A recombinant reductase gene may encode a protein having, having at least, or having at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous amino acids starting at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 of the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74.

Substrates for the reductase protein may include any fatty acid from 14 to 20 carbons long with a methylene substitution in the A9, A10, or Δ11 position. The fatty acid substrate may be 14, 15, 16, 17, 18, 19, or 20 carbons long, or any range derivable therein. The reductase protein may be capable of catalyzing the reduction of a methylene-substituted fatty acid substrate to a (methyl)lipid. The reductase protein, together with a methyltransferase protein, may be capable of catalyzing the production of a methylated branch from any fatty acid from 14 to 20 carbons long with an unsaturated double bond in the Δ9, Δ10, or Δ11 position.

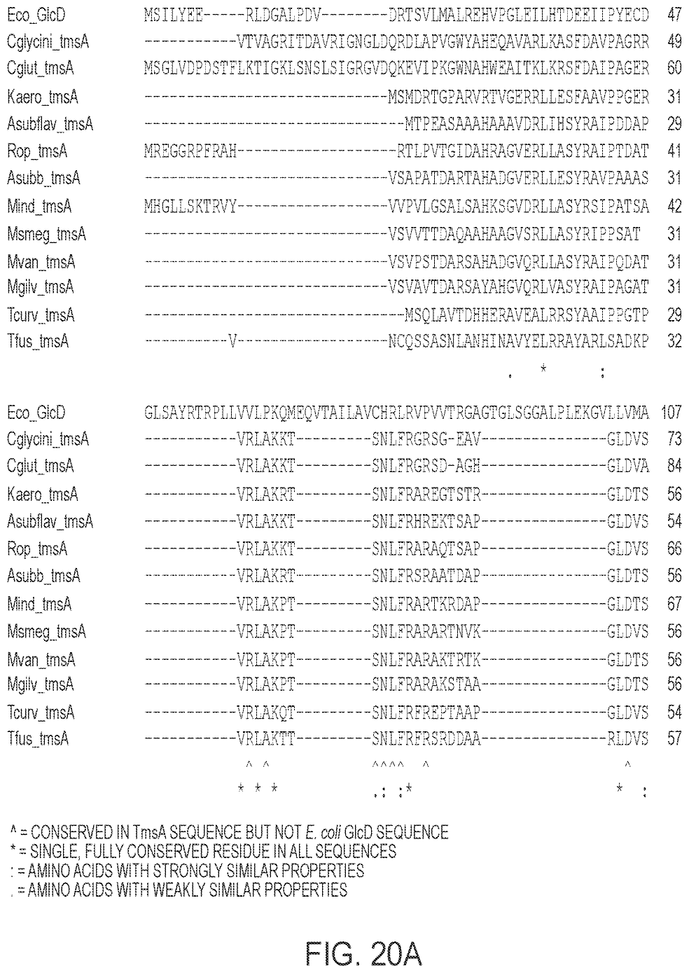

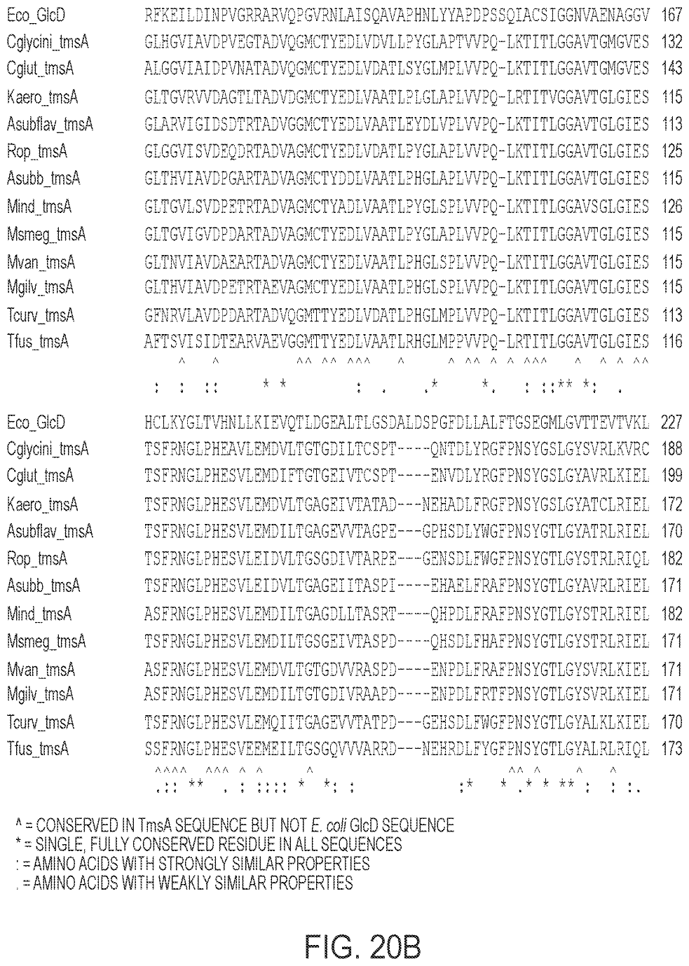

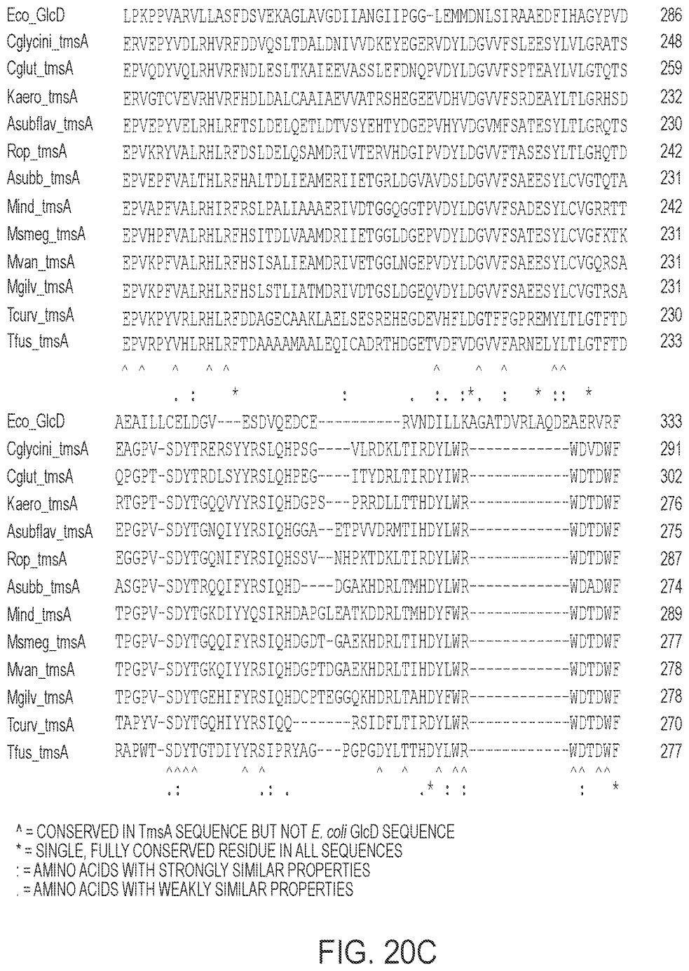

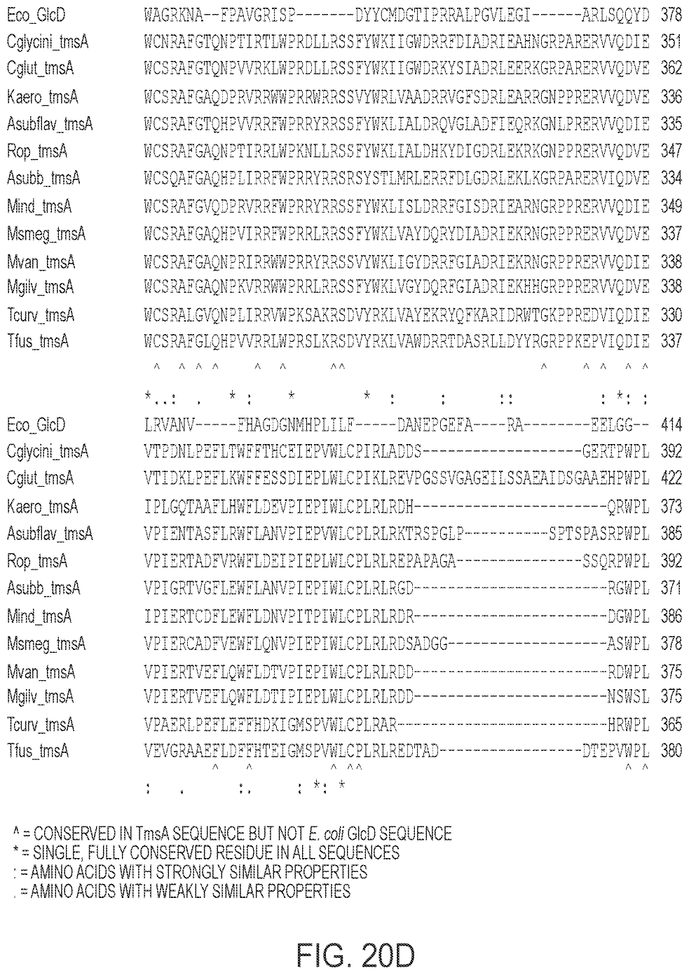

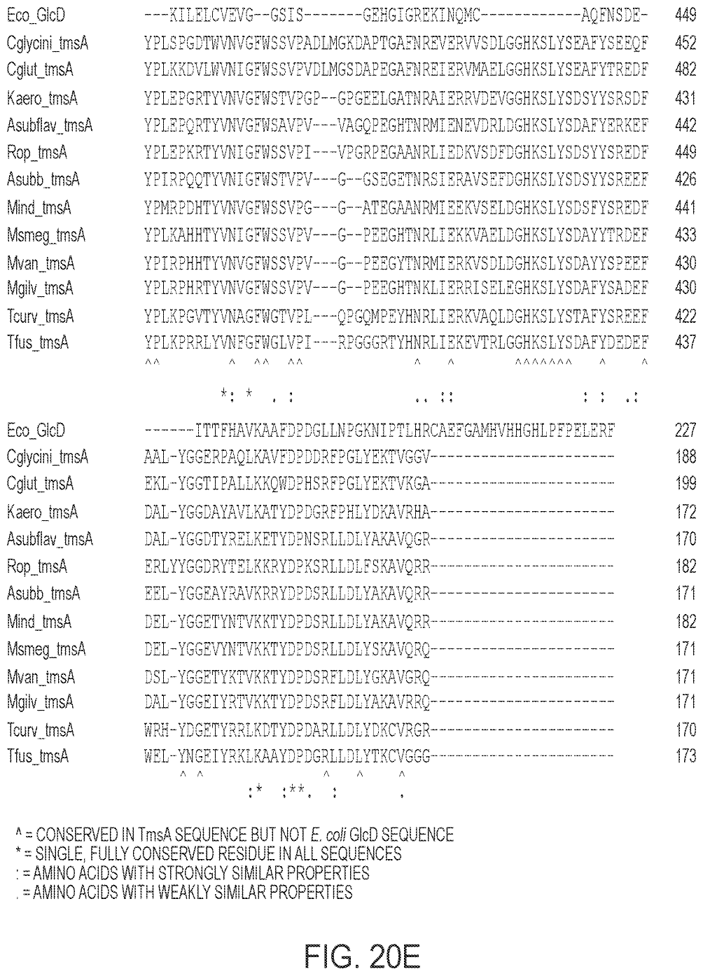

In some embodiments, the recombinant reductase gene encodes a reductase protein that includes a Flavin adenine dinucleotide (FAD) binding domain. In some embodiments, the FAD binding domain has at least 80%, 85%, 90%, 91%, 92%, 93%, 94%, 95%, 96%, 97%, 98%, 99%, 99.9%, or 100% sequence identity to amino acids 9-141 of T. curvata TmsA (SEQ ID NO:74) or to a corresponding portion of TmsA from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, or Thennobifida fusca, according to the alignment set forth in FIGS. 20A-E.

In some embodiments, the recombinant reductase gene encodes a reductase protein that includes a FAD/FMN-containing dehydrogenase domain. In some embodiments, the FAD/FMN-containing dehydrogenase domain has, has at least, or has at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to amino acids 22-444 of T. curvata TmsA (SEQ ID NO:74) or to a corresponding portion of TmsA from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, or Thennobifida fusca, according to the alignment set forth in FIGS. 20A-E.

In some embodiments, the recombinant reductase gene encodes a reductase protein that has specific amino acids unchanged from the amino acid sequence set forth in SEQ ID NO:2, SEQ ID NO:6, SEQ ID NO:10, SEQ ID NO:14, SEQ ID NO:18, SEQ ID NO:24, SEQ ID NO:28, SEQ ID NO:34, SEQ ID NO:40, SEQ ID NO:44, SEQ ID NO:48, SEQ ID NO:52, SEQ ID NO:58, SEQ ID NO:62, SEQ ID NO:68, or SEQ ID NO:74. The unchanged amino acids can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or amino acids selected from R31, A33, S37, N38, L39, F40, R43, D52, V59, D63, G73, M74, T76, Y77, D79, L80, V81, L85, P91, V93, V94, Q96, L97, T99, I100, T101, A105, G108, G110, E112, 5113, 5115, F116, R117, N118, P121, H122, E123, V125, E127, G133, P154, N155, Y157, Y162, L166, E171, V173, V177, H181, V208, G213, F216, Y222, L223, 5236, D237, Y238, T239, Y245, 5247, D254, T257, Y261, W263, R264, W265, D266, D268, W269, C272, A275, G277, Q279, R284, W287, R293, 5294, G318, E232, V325, P328, E330, F339, F343, W353, C355, P356, W363, L365, Y366, P367, N376, F379, W380, V383, P384, N395, E399, G407, H408, K409, 5410, L411, Y412, 5413, Y417, F422, Y426, G428, R443, L447, and V452 of T. curvata TmsA (SEQ ID NO:74) or corresponding amino acids in TmsA from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, or Thennobifida fusca, according to the alignment set forth in FIGS. 20A-E.

iii. Nucleic Acids Comprising a Recombinant tmsC Gene

A nucleic acid may comprise a 10-methylstearic C gene (tmsC), as described herein. A tmsC gene (e.g., a recombinant tmsC gene) may comprise any one of the nucleotide sequences set forth in SEQ ID NO:21, SEQ ID NO:31, SEQ ID NO:37, SEQ ID NO:55, SEQ ID NO:65, and SEQ ID NO:71. A tmsC gene (e.g., a recombinant tmsC gene) may be derived from a gram-positive species of Actinobacteria, such as Mycobacteria, Corynebacteria, Nocardia, Streptomyces, or Rhodococcus. A tmsC gene (e.g., a recombinant tmsC gene) may be selected from the group consisting of Corynebacterium glyciniphilium gene tmsC, Mycobacterium austroafricanum gene tmsC, Mycobacterium gilvum gene tmsC, Mycobacterium vanbaalenii gene tmsC, Streptomyces regnsis gene tmsC, and Thennobifida fusca gene tmsC.

A recombinant tmsC gene may be recombinant because it is operably-linked to a promoter other than the naturally-occurring promoter of the tmsC gene. Such genes may be useful to drive transcription in a particular species of cell. A recombinant tmsC gene may be recombinant because it contains one or more nucleotide substitutions relative to a naturally-occurring tmsC gene. Such genes may be useful to increase the translation efficiency of the tmsC gene's mRNA transcript in a particular species of cell.

A nucleic acid may comprise a recombinant tmsC gene and a promoter, wherein the recombinant tmsC gene and promoter are operably-linked. The recombinant tmsC gene and promoter may be derived from different species. For example, the recombinant tmsC gene may encode the tmsC protein of a gram-positive species of Actinobacteria, and the recombinant tmsC gene may be operably-linked to a promoter that can drive transcription in another phylum of bacteria (e.g., a Proteobacterium, such as E. coli) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise a recombinant tmsC gene, and the recombinant tmsC gene may be operably-linked to a promoter capable of driving transcription of the recombinant tmsC gene in the cell. The cell may be a species of yeast, and the promoter may be a yeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant, and the promoter may be a plant promoter.

A recombinant tmsC gene may be operably-linked to a promoter that cannot drive transcription in the cell from which the recombinant tmsC gene originated. For example, the promoter may not be capable of binding an RNA polymerase of the cell from which a recombinant tmsC gene originated. In some embodiments, the promoter cannot bind a prokaryotic RNA polymerase and/or initiate transcription mediated by a prokaryotic RNA polymerase. In some embodiments, a recombinant tmsC gene is operably-linked to a promoter that cannot drive transcription in the cell from which the protein encoded by the gene originated. For example, the promoter may not be capable of binding an RNA polymerase of a cell that naturally expresses the tmsC enzyme encoded by a recombinant tmsC gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A recombinant tmsC gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:21, SEQ ID NO:31, SEQ ID NO:37, SEQ ID NO:55, SEQ ID NO:65, or SEQ ID NO:71. A recombinant tmsC may or may not have 100% sequence identity with any one of the nucleotide sequences set forth in SEQ ID NO:21, SEQ ID NO:31, SEQ ID NO:37, SEQ ID NO:55, SEQ ID NO:65, and SEQ ID NO:71. A recombinant tmsC gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:21, SEQ ID NO:31, SEQ ID NO:37, SEQ ID NO:55, SEQ ID NO:65, and SEQ ID NO:71, and the recombinant tmsC gene may encode a tmsC protein with at least about 95%, 96%, 97%, 98%, 99%, or 100% sequence identity with the amino acid sequence set forth in SEQ ID NO:22, SEQ ID NO:32, SEQ ID NO:38, SEQ ID NO:56, SEQ ID NO:66, and SEQ ID NO:72.

A recombinant tmsC gene may vary from a naturally-occurring tmsC gene because the recombinant tmsC gene may be codon-optimized for expression in a eukaryotic cell, such as a plant cell, algae cell, or yeast cell. A cell may comprise a recombinant tmsC gene, wherein the recombinant tmsC gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant tmsC gene may vary from a naturally-occurring tmsC gene or may remain unchanged from a naturally-occurring tmsC gene. For example, a recombinant tmsC gene may comprise a nucleotide sequence with at least about 65% sequence identity with the naturally-occurring nucleotide sequence set forth in SEQ ID NO:21, SEQ ID NO:31, SEQ ID NO:37, SEQ ID NO:55, SEQ ID NO:65, or SEQ ID NO:71 (e.g., at least about 70%, 75%, 80%, 85%, 90%, 95%, 96%, 97%, 98%, or 99% sequence identity), and at least 5 codons of the nucleotide sequence of the recombinant tmsC gene may vary from the naturally-occurring nucleotide sequence (e.g., at least about 10, 15, 20, 25, 30, 35, 40, 45, 50, 60, 70, 80, 90, or 100 codons).

A tmsC gene encodes a tmsC protein. A tmsC protein may be a protein expressed by a gram-positive species of Actinobacteria, such as Mycobacteria, Corynebacteria, Nocardia, Streptomyces, or Rhodococcus. A recombinant tmsC gene may encode a naturally-occurring tmsC protein even if the recombinant tmsC gene is not a naturally-occurring tmsC gene. For example, a recombinant tmsC gene may vary from a naturally-occurring tmsC gene because the recombinant tmsC gene is codon-optimized for expression in a specific cell. The codon-optimized, recombinant tmsC gene and the naturally-occurring tmsC gene may nevertheless encode the same naturally-occurring tmsC protein.

A recombinant tmsC gene may encode a tmsC protein selected from Corynebacterium glyciniphilium enzyme tmsC, Mycobacterium austroafricanum enzyme tmsC, Mycobacterium gilvum enzyme tmsC, Mycobacterium vanbaalenii enzyme tmsC, Streptomyces regnsis enzyme tmsC, and Thennobifida fusca enzyme tmsC. A recombinant tmsC gene may encode a tmsC protein, and the tmsC protein may be substantially identical to any one of the foregoing enzymes, but the recombinant tmsC gene may vary from the naturally-occurring gene that encodes the enzyme. The recombinant tmsC gene may vary from the naturally-occurring gene because the recombinant tmsC gene may be codon-optimized for expression in a specific phylum, class, order, family, genus, species, or strain of cell.

The sequences of naturally-occurring tmsC proteins are set forth in SEQ ID NO:22, SEQ ID NO:32, SEQ ID NO:38, SEQ ID NO:56, SEQ ID NO:66, and SEQ ID NO:72. A recombinant tmsC gene may or may not encode a protein comprising 100% sequence identity with the amino acid sequence set forth in SEQ ID NO:22, SEQ ID NO:32, SEQ ID NO:38, SEQ ID NO:56, SEQ ID NO:66, and SEQ ID NO:72. For example, a recombinant tmsC gene may encode a protein having 100% sequence identity with a biologically-active portion of an amino acid sequence set forth in SEQ ID NO:22, SEQ ID NO:32, SEQ ID NO:38, SEQ ID NO:38, SEQ ID NO:56, SEQ ID NO:66, and SEQ ID NO:72. A recombinant tmsC gene may encode a tmsC protein having at least about 95%, 96%, 97%, 98%, or 99% sequence identity with the amino acid sequence set forth in SEQ ID NO:22, SEQ ID NO:32, SEQ ID NO:38, SEQ ID NO:56, SEQ ID NO:66, or SEQ ID NO:72, or a biologically-active portion thereof.

iv. Nucleic Acids Comprising a Recombinant Methyltransferase Gene and a Recombinant Reductase Gene

A nucleic acid may comprise both a recombinant methyltransferase gene and a recombinant reductase gene. The recombinant methyltransferase gene and the recombinant reductase gene may encode proteins from the same species or from different species. A nucleic acid may comprise a recombinant methyltransferase gene, a recombinant reductase gene, and/or a tmsC gene. A recombinant methyltransferase gene, recombinant reductase gene, and a tmsC gene may encode proteins from 1, 2, or 3 different species (i.e., the genes may each be from the same species, two genes may be from the same species, or all three genes may be from different species).

A nucleic acid may comprise the nucleotide sequence set forth in SEQ ID NO:77, SEQ ID NO:78, or SEQ ID NO:79. A nucleic acid may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:77, SEQ ID NO:78, SEQ ID NO:79, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, or SEQ ID NO:92.

In some embodiments, the nucleic acid encodes a fusion protein that includes both a methyltransferase and a reductase or fragments thereof. In the context of the present invention, “fusion protein” means a single protein molecule containing two or more distinct proteins or fragments thereof, covalently linked via peptide bond in a single peptide chain. In some embodiments, the fusion protein comprises enzymatically active domains from both a methyltransferase protein and a reductase protein. The nucleic acid may further encode a linker peptide between the methyltransferase and the reductase. In some embodiments, the linker peptide comprises the amino acid sequence AGGAEGGNGGGA (SEQ ID NO: 99). The linker may comprise about or at least about 2, 3, 4, 5, 6, 7, 8, 9, 10, 15, 20, 25, or 30 amino acids, or any range derivable therein. The nucleic acid may comprise any of the methyltransferase and reductase genes described herein, and the fusion protein encoded by the nucleic acid can comprise any of the methyltransferase and reductase proteins described herein, including biologically active fragments thereof. In some embodiments, the fusion protein is a tmsA-B protein, in which the TmsA protein is closer to the N-terminus than the TmsB protein. An example of such a tmsA-B protein is encoded by the nucleic acid sequence of SEQ ID NO:97. In some embodiments, the fusion protein is a tmsB-A protein, in which the tmsB protein is closer to the N-terminus than the tmsA protein. An example of such a tmsB-A protein is encoded by the nucleic acid sequence of SEQ ID NO:98. In some embodiments, the fusion protein has at least about 80%, 85%, 90%, 91%, 92%, 93%, 94%, 95%, 96%, 97%, 98%, 99%, or 99.9% identity to the amino acid sequence of a fusion protein encoded by SEQ ID NO:97 or SEQ ID NO:98.

### C. Compositions

Various aspects of the invention relate to compositions produced by the cells described herein. The composition may be an oil composition comprised of about or at least about 75%, 80%, 85%, 90%, 95%, or 99% lipids. The composition may comprise branched (methyl)lipids and/or exomethylene-substituted lipids. The branched (methyl)lipid may be a carboxylic acid (e.g., 10-methylstearic acid, 10-methylpalmitic acid, 12-methyloleic acid, 13-methyloleic acid, 10-methyl-octadec-12-enoic acid), carboxylate (e.g., 10-methylstearate, 10-methylpalmitate, 12-methyloleate, 13-methyloleate, 10-methyl-octadec-12-enoate), ester (e.g., diacylglycerol, triacylglycerol, phospholipid), thioester (e.g., 10-methylstearyl CoA, 10-methylpalmityl CoA, 12-methyloleoyl CoA, 13-methyloleoyl CoA, 10-methyl-octadec-12-enoyl CoA), or amide. The exomethylene-substituted lipid may be a carboxylic acid (e.g., 10-methylenestearic acid, 10-methylenepalmitic acid, 12-methyleneoleic acid, 13-methyleneoleic acid, 10-methylene-octadec-12-enoic acid), carboxylate (e.g., 10-methylenestearate, 10-methylenepalmitate, 12-methyleneoleate, 13-methyleneoleate, 10-methylene-octadec-12-enoate), ester (e.g., diacylglycerol, triacylglycerol, phospholipid), thioester (e.g., 10-methylenestearyl CoA, 10-methylenepalmityl CoA, 12-methyleneoleoyl CoA, 13-methyleneoleoyl CoA, 10-methylene-octadec-12-enoyl CoA), or amide. 10-methyl lipids, 10-methylene lipids, or both. It is specifically contemplated that one or more of the above lipids may be excluded from certain embodiments.

In some aspects, the composition is produced by cultivating a culture comprising any of the cells described herein and recovering the oil composition from the cell culture. The cells in the culture may contain any of the recombinant methyltransferase genes described herein and/or any of the recombinant reductase genes described herein. The culture medium and conditions can be chosen based on the species of the cell to be cultured and can be optimized to provide for maximal production of the desired lipid profile.

Various methods are known for recovering an oil composition from a culture of cells. For example, lipids, lipid derivatives, and hydrocarbons can be extracted with a hydrophobic solvent such as hexane. Lipids and lipid derivatives can also be extracted using liquefaction, oil liquefaction, and supercritical CO₂ extraction. The recovery process may include harvesting cultured cells, such as by filtration or centrifugation, lysing cells to create a lysate, and extracting the lipid/hydrocarbon components using a hydrophobic solvent.

In addition to accumulating within cells, the lipids described herein may be secreted by the cells. In that case, a process for recovering the lipid may not require creating a lysate from the cells, but collecting the secreted lipid from the culture medium. Thus, the compositions described herein may be made by culturing a cell that secretes one of the lipids described herein, such as a a linear fatty acid with a chain length of 14-20 carbons with a methyl branch at the Δ9, Δ10, or Δ11 position.

In some embodiments, the oil composition comprises about, at least about, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of a branched (methyl)lipid, such as a 10-methyl fatty acid, or any range derivable therein. In some embodiments, 10-methyl fatty acids comprise about, at least about, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of the fatty acids in the composition, or any range derivable therein.

### D. Methods of Producing a Branched (Methyl)Lipid

Various aspects of the invention relate to a method of producing a branched (methyl)lipid. The method may comprise incubating a cell or plurality of cells as described herein, supra, with media. The media may optionally be supplemented with an unbranched, unsaturated fatty acid, such as oleic acid, that serves as a substrate for methylation. The media may optionally be supplemented with methionine or s-adenosyl methionine, which may similarly serve as a substrate. Thus, the method may comprise contacting a cell or plurality of cells with oleic acid, methionine, or both. The method may comprise incubating a cell or plurality of cells as described herein, supra, in a bioreactor. The method may comprise recovering lipids from the cells and/or from the culture medium, such as by extraction with an organic solvent.

The method may comprise degumming the cell or plurality of cells, e.g., to remove proteins. The method may comprise transesterification or esterification of the lipids of the cells. An alcohol such as methanol or ethanol may be used for transesterification or esterification, e.g., thereby producing a fatty acid methyl ester or fatty acid ethyl ester.

### EXEMPLIFICATION

The present description is further illustrated by the following examples, which should not be construed as limiting in any way.

### Example 1: Identification of 10-Methylstearic Genes tmsA, tmsB, and tmsC

Two different genes have been identified as responsible for 10-methylstearate production in M. tuberculosis (see Meena, L. S., and P. E. Kolattukudy, BIOTECHNOLOGY & APPLIED BIOCHEMISTRY 60(4):412 (2013) and Meena, L. S., et al. BIOLOGICAL CHEMISTRY 394(7):871 (2013)). Curiously, neither gene is conserved throughout each Actinobacteria species that produces 10-methylstearate. While it is possible that different species of Actinobacteria each independently evolved genes that synthesize 10-methylstearate, such convergent evolution is rare. A simpler explanation is that a single common gene or set of genes is responsible for 10-methylstearate production in Actinobacteria.

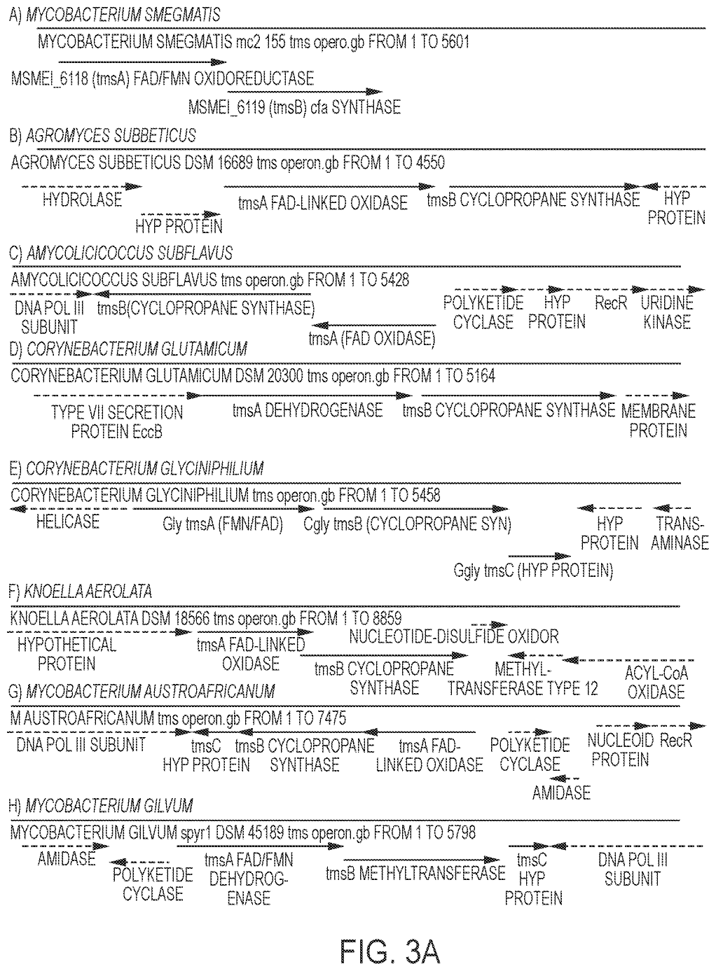

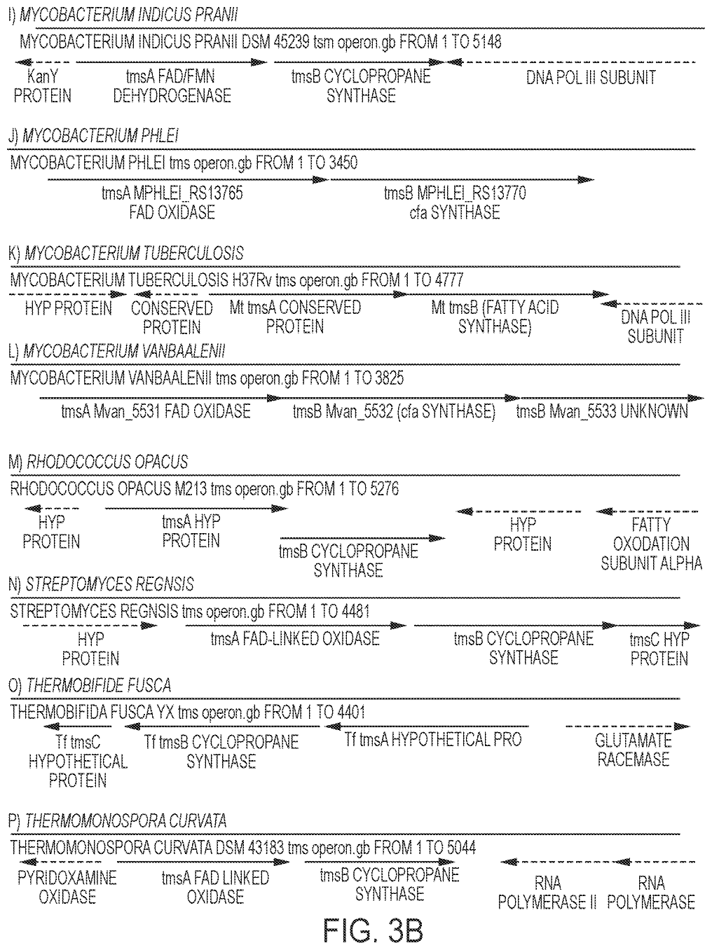

To identify genes that may be responsible for 10-methylstearate production in Actinobacteria, genes with sequence homology to those that encode enzymes that catalyze lipid synthesis reactions were aligned from various species of 10-methylstearate-producing Actinobacteria. Two unique genes were identified and named 10-methystearic A (tmsA) and 10-methylstearic B (tmsB), which each occur in the same operon within each 10-methystearate producing species of Actinobacteria (FIG. 3). A third gene named 10-methylstearic C (tmsC) was identified as occurring in the same operon as tmsA and tmsB for some of the 10-methylstearate-producing species.

The 10-methylstearate B gene has sequence homology with cyclopropane synthases, which suggests that the 10-methylstearate B gene may be capable of transferring a methyl group to a fatty acid. The 10-methylstearic A gene has sequence homology with oxidoreductases, which suggests that it may be capable of reducing the exomethylene group of a branched fatty acid.

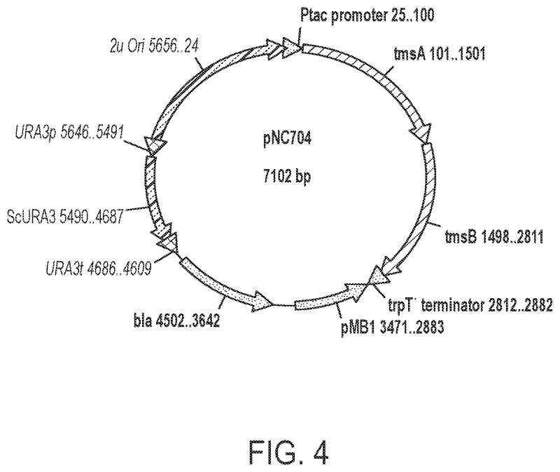

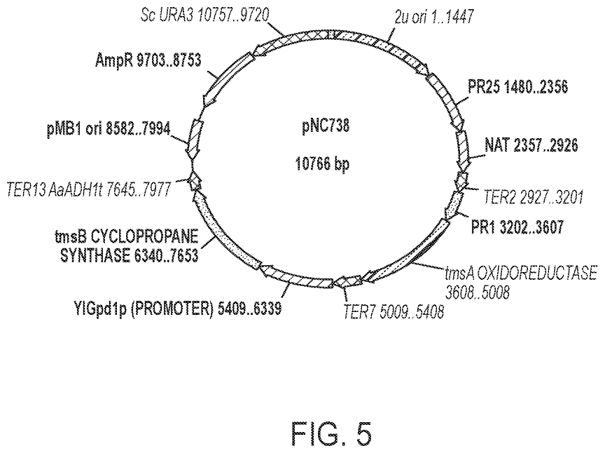

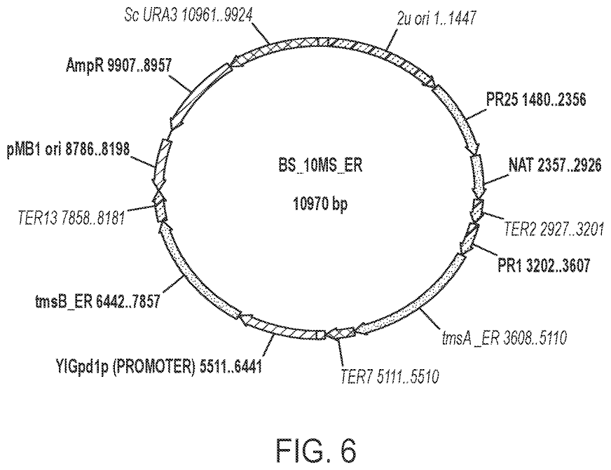

The 10-methylstearate A and 10-methylstearate B genes from M. smegmatis were cloned into a plasmid (named pNC704) for expression in E. coli (FIG. 4). The pNC704 plasmid harboring M. smegmatis tmsA and tmsB was used to transform E. coli. The transformed cells were grown for 20 hours at 37° C. in LB media supplemented with 100 μg/mL oleic acid. E. coli was transformed with an empty vector pNC53 (SEQ ID NO:81) and grown in parallel as a control. Each of two E. coli colonies transformed with pNC704 produced 10-methylstearate at a concentration of 2.0% and 2.1% of the total fatty acids in the cell (Table 1). The control did not produce 10-methylstearate

TABLE 1
Fatty acid concentration as a percentage of total cellular fatty acids.
“10-MS” corresponds to 10-methylstearate

| | Fatty acid composition | | | | |
|---|---|---|---|---|---|
| | % | % | | | |
| | 10-MS | 16:1 | % 16:0 | % 18:0 | % 18:1 |
| E. coli TOP10 + pNC53 | 0.0 | 4.0 | 56.8 | 1.4 | 30.6 |
| E. coli TOP10 + pNC704 | 2.1 | 4.2 | 55.0 | 0.8 | 30.9 |
| isolate 1 | | | | | |
| E. coli TOP10 + pNC704 | 2.0 | 3.9 | 55.5 | 0.8 | 30.8 |
| isolate 2 | | | | | |

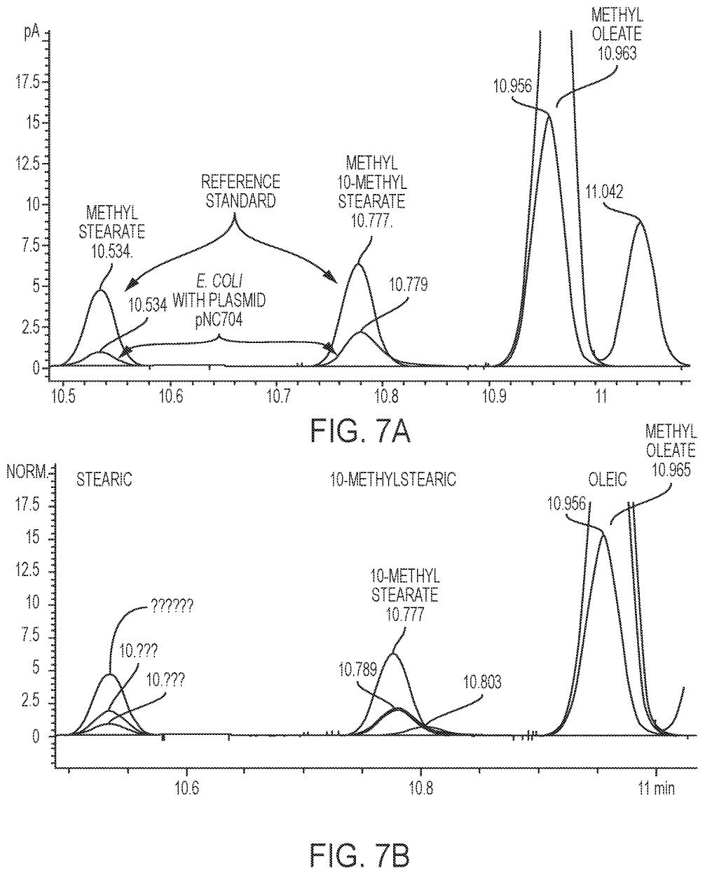

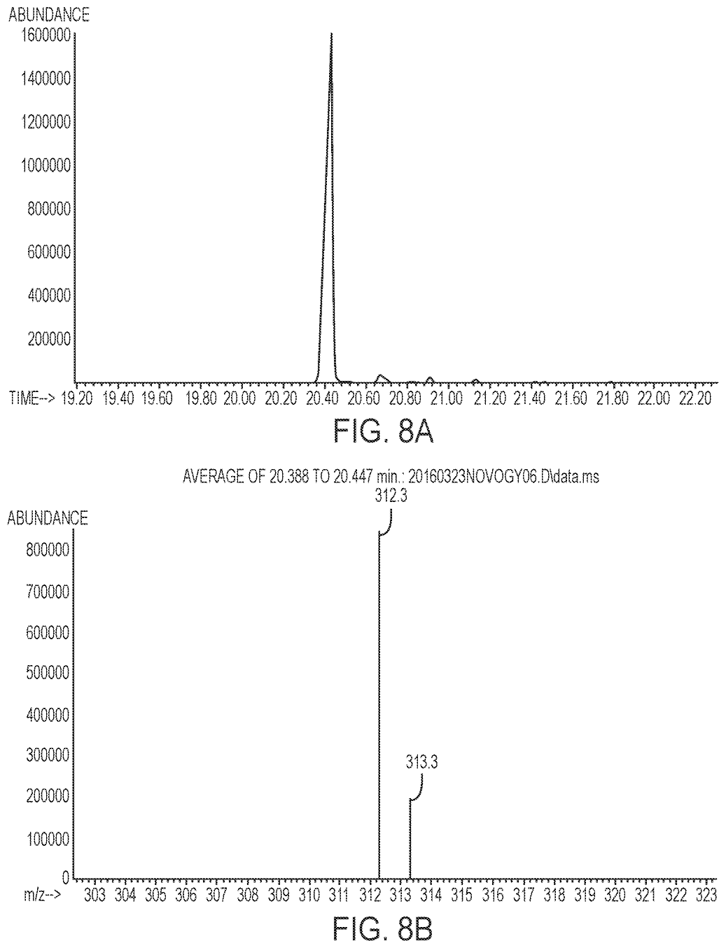

Cellular lipids were transesterified to produce fatty acid methyl esters (FAMEs) in a solution of HCl in methanol. Stearic acid, 10-methylstearic acid, and oleic acid were transesterified into FAMEs as standards. Each sample/standard was extracted into isooctane and analyzed by various gas chromatography methods (FIGS. 7 and 8). FAMEs were first analyzed by capillary gas chromatography using a flame-ionization detector (GC-FID). The FAMEs produced from E. coli displayed a GC peak corresponding to the 10-methylstearic acid FAME standard, which suggests that the M. smegmatis tmsA and tmsB genes express proteins that are capable of synthesizing 10-methylstearic acid (FIG. 7A).

FAMEs were also produced from E. coli that was transformed with the empty vector pNC53 and analyzed by GC-FID as above. This sample did not display a GC peak corresponding to the 10-methylstearic acid FAME, further suggesting that the M. smegmatis tmsA and tmsB genes express proteins that are capable of synthesizing 10-methylstearic acid (FIG. 7B).

The FAMEs produced from the tmsA/tmsB sample were analyzed using a GC-MS configured in single-ion monitoring mode (SIM), which monitored m/z at 312.3 and 313.3 amu. The mass spectrum displayed a peak at 312.3 amu, corresponding to the molecular weight of a 10-methylstearate methyl ester (FIG. 8B). Additionally, the ratio of the peak at 312.3 amu to 313.3 amu suggests that the ion observed at 312.3 amu contains 20.6 carbons, which corresponds to the actual number of carbons (20) in the 10-methylstearate methyl ester.

### Example 2: Production of 10-Methyl Fatty Acid in E. coli Using tmsB and tmsA Genes from Different Donor Organisms

Methods:

Donor bacteria genomic DNA was obtained from Deutsche Sammlung von Mikroorganismen and Zellkulturen (DSMZ), Germany. Plasmids were constructed with standard molecular biology techniques using the “yeast gap repair” method (Shanks, et al., Appl. Microbiol. Biotechnol., 48:232 (1997)). The empty E. coli expression vector pNC53 (SEQ ID NO:82) was restriction digested with enzyme Pmel (New England Biolabs, MA), creating a double strand break between the tac promoter and trpT′ terminator sequences on this vector. tmsAB gene operons were PCR amplified from genomic DNA with primer flanking sequence such that the tmsB ATG start site integrated into the end of the tac promoter via homologous recombination. E. coli transcription and translation was driven by the tac promoter. The stop codon of the tmsA gene similarly integrated into the beginning of the trpT′ terminator region. E. coli translation of the operon-embedded tmsA gene relied on native translation signals from the donor organism DNA. Where necessary, the first codon of tmsB was altered from GTG or TTG to ATG; otherwise the native codon sequence was kept in the E. coli expression vectors.

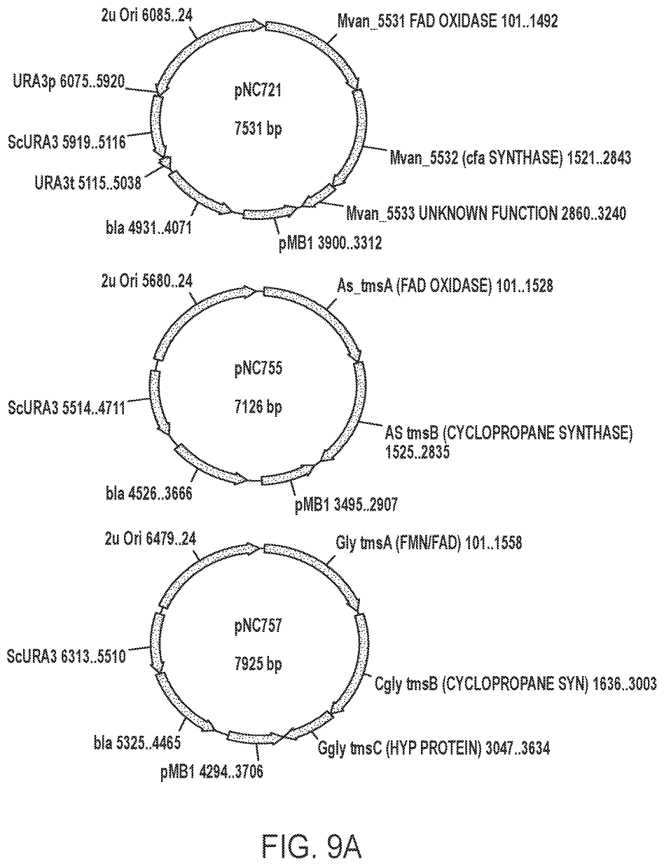

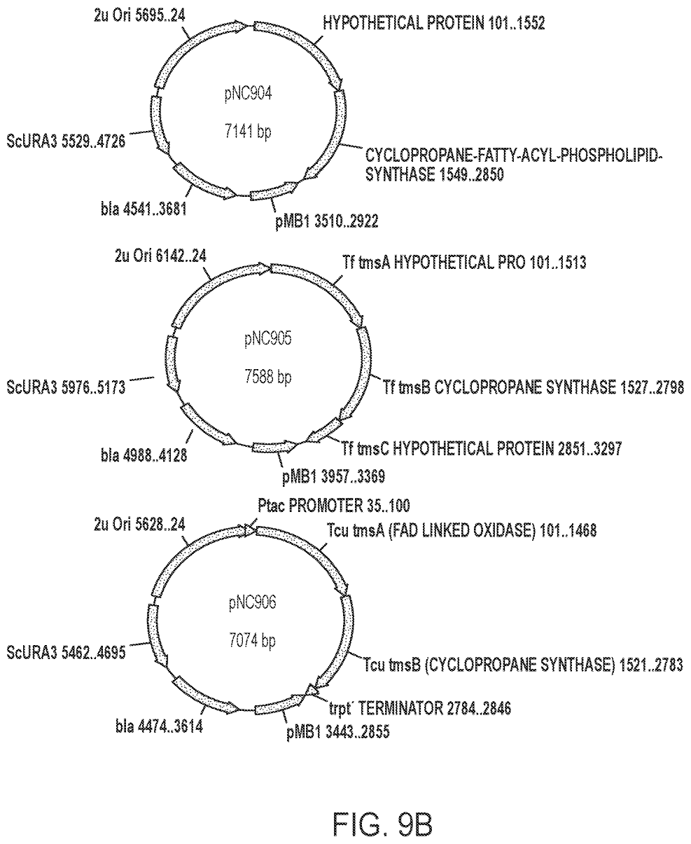

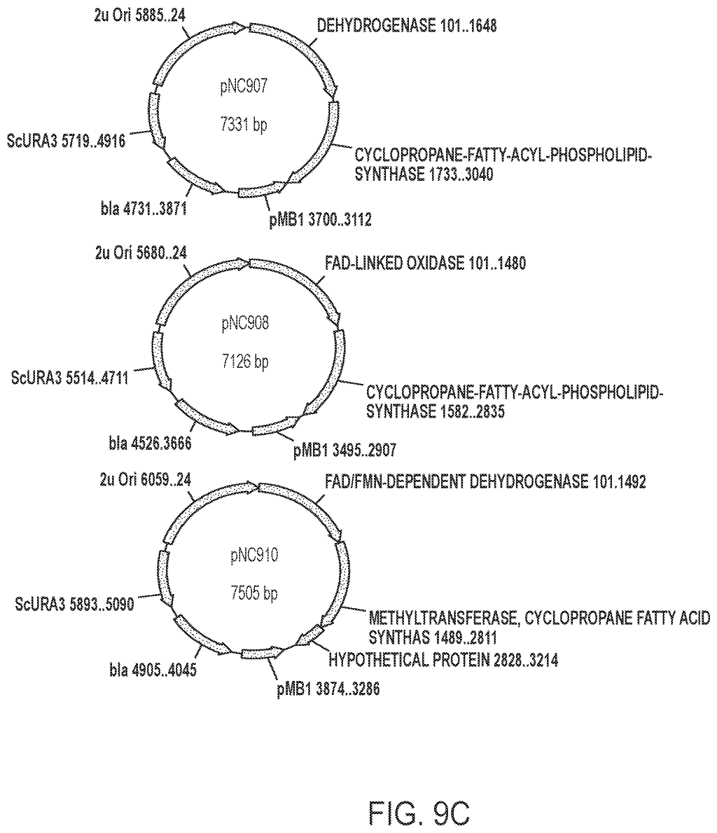

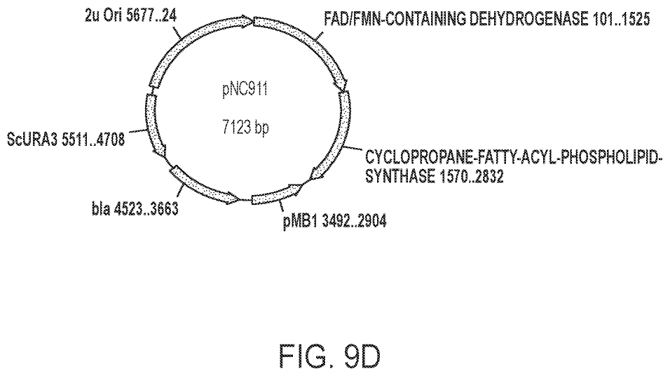

Vectors were checked by DNA sequencing and restriction digest for correct construction. The vectors created for this example are illustrated in FIG. 9. Vectors transformed into E. coli Top10 (Invitrogen) were then used for fermentation studies. Cells were inoculated in 50 mL LB medium supplemented with 100 mg/L ampicillin and 100 mg/L oleic acid from a stock solution of 100 mg/mL oleic acid in ethanol. Cultures were incubated at 37° C. and 200 rpm in baffled shake flasks for 41 hours. At the end of cultivation, cells were harvested by centrifugation at 4000 rpm for 15 minutes in an Eppendorf 5810 R clinical centrifuge, washed once with and equal volume of deionized water, resuspended in 0.1 mL deionized water, and frozen at −80° C. Cells were then lyophilized to dryness and used to perform an acid-catalyzed transesterification with a solution of 0.5 N HCl in methanol (20×1 mL ampule, Sigma) at 85° C. for 90 minutes. After the transesterification was completed, the lipid-soluble components of the reaction mixture were separated from the water-soluble components using a two-phase liquid extraction by adding water and isooctane and subsequently analyzed with a capillary gas chromatograph (GC) equipped with a robotic injector, flame ionization detector (Agilent Technologies 7890B GC system and 7396 Autosampler) and HP-INNOWAX capillary column (30 m×0.25 mm×0.15 micrometers, Agilent). A 10-methylstearic acid reference standard was obtained from Larodan AB, Sweden.

### Results

Conversion of oleic acid to 10-methylstearic acid was observed for 4 of the 11 vectors tested. Highest percent conversion occurred with tmsAB genes from Thermobifida fusca (22%) and Thermomonospora curvata (38%), as indicated in Table 2 below.

TABLE 2

| | | | % oleic acid |
|---|---|---|---|
| | | | conversion |
| | | | to 10- |
| E. coli | | | methylstearic |
| vector | Sequence | Donor organism | acid |
| pNC704 | SEQ ID NO: 77 | Mycobacterium smegmatis | 4.9% ± 0.6% |
| pNC721 | SEQ ID NO: 83 | Mycobacterium vanbaaleni | 0 |
| pNC755 | SEQ ID NO: 84 | Amycolicicoccus subflavus | 0 |
| pNC757 | SEQ ID NO: 85 | Corynebacterium | 0 |
| | | glyciniphilum | |
| pNC904 | SEQ ID NO: 86 | Rhodococcus opacus | 1.2% ± 0.2% |
| pNC905 | SEQ ID NO: 87 | Thermobifida fusca | 22.0% ± 0.3% |
| pNC906 | SEQ ID NO: 88 | Thermomonospora curvata | 38.3% ± 0.5% |
| pNC907 | SEQ ID NO: 89 | Corynebacterium | 0 |
| | | glutamicum | |
| pNC908 | SEQ ID NO: 90 | Agromyces subbeticus | 0 |
| pNC910 | SEQ ID NO: 91 | Mycobacterium gilvum | 0 |
| pNC911 | SEQ ID NO: 92 | Mycobacterium sp. indicus | 0 |

### Example 3: tmsB and tmsA Expression in Rhocococcus Opacus PD630

The oleaginous bacteria Rhocococcus opacus can produce 10-methyl fatty acids natively at low levels (0.2% of total fatty acids (Waltermann et al., Microbiology, 72:5027 (2006)), and additionally possesses native homologs of the tmsB and tmsA gens, although they have not been identified as such in the literature. In this Example, the inventors tested whether overexpression of the tmsB and tmsA genes in R. opacus can increase 10-methyl branched fatty acid content.

Methods:

Rhodococcus opacus PD630 was obtained from the German Collection of Microorganisms and Cell Cultures (DSMZ) from stock DSM 44193. The culture was revived by dilution with 4 mL LB media and incubated at 30° C. for 3 days in a drum roller. Once visible growth occurred, 10 μL broth was struck to single colonies on an LB plate and incubated an additional 3 days at 30° C. One colony was isolated and designated strain NS1104.

All R. opacus growth was performed at 30° C. Routine culturing was performed in LB medium supplemented with appropriate antibiotics. Genetic transformation was performed in Nutrient Broth medium as modified by Kalscheuer et al. (Appl. Microbiol. and Biotechnol., 52:508 (1999)), which contained 5 g/L peptone, 2 g/L yeast extract, 1 g/L beef extract, 5 g/L NaCl, 8.5 g/L glycine, and 10 g/L sucrose. Lipid production was performed in defined medium containing the following components and adjusted to pH 7.6 with NaOH and filter sterilized before use.

R. opacus fermentation medium

| Component | g/L |
|---|---|
| Glucose | 40 |
| (NH₄)₂SO₄ | 1.4 |
| MgSO₄•7H₂O | 1 |
| CaCl₂•6H₂O | 0.02 |
| KH₂PO₄ | 0.4 |
| MOPS acid | 5 |
| Trace element solution | 1 mL |
| Trace element solution | g/L stock solution |
| FeSO₄•7H₂O | 0.5 |
| CuSO₄•5H₂O | 0.005 |
| ZnSO₄•7H₂O | 0.4 |
| MnCl₂•2H₂O | 0.02 |
| Na₂MoO₄•2H₂O | 0.02 |
| CoCl₂•6H₂O | 0.05 |
| EDTA | 0.25 |
| H₃BO₃ | 0.015 |
| NiCl₂•6H₂O | 0.01 |

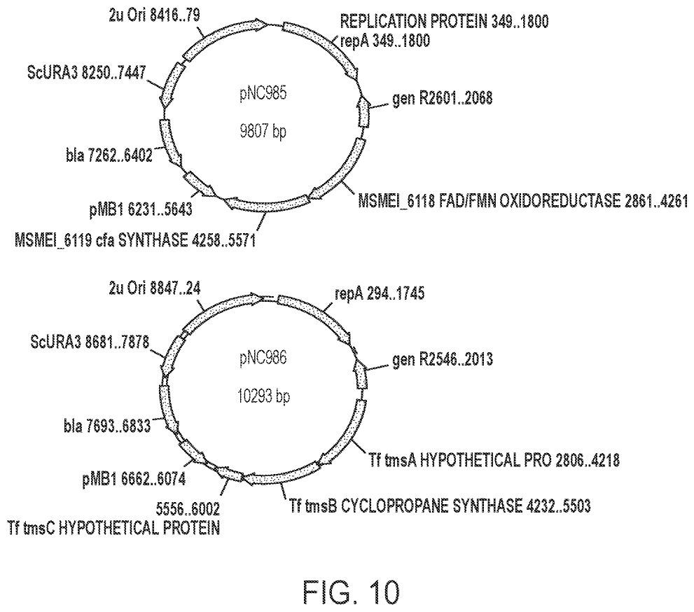

Plasmids were constructed with standard molecular biology techniques using the “yeast gap repair” method (Shanks et al., Applied and Environmental Biology 72:5207-36 (2006)). A synthetic DNA sequence containing the Rhodococcus repA origin of replication and gentamicin resistance marker (Lessard, BMC Microbiol., 4:15 (2004)) was used to create a R. opacus-E. coli-S. cerevisiae shuttle vector from two plasmids containing the tmsAB genes from Mycobacterium smegmatis and Thermobifida fusca under control of the tac promoter. Briefly, the repA and gen R synthetic DNA was constructed with approximately 50 bp flanking homology regions to the tmsAB destination plasmids. Destination plasmids were restriction digested with PacI, and the flanking homology regions repaired the gap, enabling genetic selection via the ura3 gene in S. cerevisiae. DNA was isolated from S. cerevisiae by phenol/chloroform extraction and ethanol precipitation and used to transform E. coli. Correct plasmid constructions were isolated by mini-prep (Qiagen, USA) and screened by restriction digest. Plasmids pNC985 (SEQ ID NO:93), containing M. smegmatis tmsAB, and pNC986 (SEQ ID NO:94) (FIG. 10), containing T. fusca tmsAB were isolated and used to transform R. opacus.

R. opacus was transformed following the protocol described by Kalscheuer et al. (Kalscheuer 1999). Cells were grown overnight in modified nutrient broth, then transferred to 50 mL modified nutrient broth medium at a starting optical density of 0.13. Cells were harvested at OD 0.36, washed twice in 50 mL ice cold water, and resuspended in 1.7 mL ice cold water. Cells were then subdivided to 350 μL volumes and 2 μL plasmid DNA at 400-600 ng/μL concentration. Cells plus DNA were incubated at 39° C. for 5 minutes immediately prior to cooling on ice and electrotransformation. Electric pulses were delivered using 2 mm gap cuvettes with a 2 kV pulse (600 Ω, 25 μF, 12 ms time constant). Cells were then diluted with 600 μL SOC medium and incubated overnight at 30° C. 200 μL overnight cell broth was then plated on LB agar containing 10 μg/mL gentamicin and incubated an additional 4 days at 30° C. for colony formation. Gentamicin resistant colonies were picked for further analysis, no resistant colonies were seen on control plates without added plasmid DNA.

Fermentation was performed at 30° C. for 4 days in 250 mL shake flasks (25 mL working volume with defined medium, 10 μg/mL gentamicin added as appropriate) at 200 rpm. Inoculum was prepared from 48 hour grown cultures in LB+10 μg/mL gentamicin. Inoculation amount was 1:25 v/v of the final volume. At the end of fermentation cells were harvested and resuspended in 1 mL distilled water and frozen at −80° C. After freezing, cells were lyophilized to dryness and then whole cells were transesterified in situ with methanolic HCl at 80° C. before extraction into isooctane and quantification by gas chromatography with flame ionization detection.

Results:

R. opacus was transformed with two vectors, pNC985 expressing the M. smegmatis tmsAB genes, and pNC986 expressing the T. fusca tmsAB genes. As shown in Table 3 below, one isolate of the pNC986 transformation, strain NS1155, produced 10-methylstearic acid at 7.2% by weight of total fatty acids, as compared to the control strain NS1104 at 3.6% by weight of total fatty acids.

TABLE 3
Weight percent 10-methylstearic acid measured in R. opacus
strains transformed with tmsAB expression vectors.

| | 10-methylstearic |
|---|---|
| Description | acid (% of total FA) |
| R. opacus PD630 (NS1104) | 3.6 |
| R. opacus + pNC985 #1 (Msm tmsAB) | 3.9 |
| R. opacus + pNC985 #2 | 3.3 |
| R. opacus + pNC985 #3 | 3.3 |
| R. opacus + pNC986 #1 (Tfu tmsAB) | 7.2 |
| R. opacus + pNC986 #2 | 3.0 |
| R. opacus + pNC986 #3 | 3.1 |

### Example 4: Acyl Chain Substrate Range for tmsB and tmsA

The inventors performed the following experiments to determine the acyl-chain substrate range of the tmsB and tmsA enzymes from Thermomonospora curvata, particularly the fatty acid chain length and double bond position.

Methods:

Unsaturated fatty acids were purchased from Nu-Check Prep, Inc., Elysian MN. Fatty acids were dissolved in DMSO at a concentration of 100 mg/mL, with the exceptions of palmitoleic acid, oleic acid, and vaccenic acid, which were dissolved in ethanol at a concentration of 100 mg/mL. A 10-methyl stearic acid reference standard was obtained from Larodan AB, Sweden.

E. coli strains NS1161 and NS1162 were used in this experiment; strain NS1161 was constructed by transforming the control (empty) vector plasmid into E. coli CGSC 9407 (aka JW1653-1 Keio collection) which holds a kan R disruption of the native E. coli cyclopropane fatty acid synthase (cfa) gene. Strain NS1162 was constructed by transforming plasmid pNC906 (SEQ ID NO:88) (FIG. 9B), containing the T. curvata tmsB and tmsA genes under control of the constitutive tac promoter, into E. coli CGSC 9407.

E. coli strains were grown in LB media supplemented with 100 mg/L ampicillin and 100 mg/L of fatty acid. Cultures were inoculated with a 1:1000 dilution of overnight pre-culture and grown in 14 mL plastic culture tubes with a 5 mL working volume at 37° C. in a rotary drum roller for 24 hours. At the end of cultivation cells were harvested by centrifugation at 4000 rpm for 15 minutes in an Eppendorf 5810 R clinical centrifuge, washed once with and equal volume of deionized water, resuspended in 0.1 mL deionized water, and frozen at −80° C. Cells were then lyophilized to dryness and used to perform a HCl-methanol catalyzed transesterification reaction to produce fatty acid methyl esters (FAME). These samples were dissolved in isooctane and injected into a gas chromatography system (Agilent Technologies) equipped with a flame ionization detector.

Results

When fed exogenous free fatty acids, E. coli can incorporate them into its phospholipids and other lipid structures. Strains NS1161 and NS1162 were cultured with 18 different unsaturated fatty acids and in a control medium with no fatty acid supplementation, and FAME profiles for the two strains were compared. To identify new unsaturated fatty acids, a GC peak corresponding to the supplemented fatty acid was identified via the strain NS1161 FAME profile as compared to the un-supplemented reference culture. and then the strain NS1162 FAME profile was checked for the same GC peak, and a new peak at a characteristic retention time shift (0.24 to 0.08 minutes forward, with the relative shift decreasing as overall retention time increases) corresponding to a methylated fatty acid. A 10-methyl stearic acid reference standard (Larodan AB, Sweden) was used as a control to assign retention time to 10-methylstearic acid.

As observed in Table 4 below, methylation occurred on fatty acids with 14, 15, 16, 17, 18, 19 and 20 carbons, and on Δ9, Δ10, and Δ11 double bond positions. The highest percent conversion to methylated fatty acids occurred at 16 and 18 carbon fatty acids at the Δ9 and Δ11 positions.

TABLE 4

| | | | Methyl- | |
|---|---|---|---|---|
| | | Unsaturated | branched FA | % conversion to |
| | | FA Retention | retention time | methyl branched |
| Fatty acid | Name | time (min) | (min) | FA |
| 12:1Δ11 | 11-Dodecenoic acid | 4.627 | — | 0.0% |
| 13:1Δ12 | 12-Tridecenoic acid | 5.765 | — | 0.0% |
| 14:1Δ9 | Myristoleic acid | 6.785 | 6.546 | 3.4% |
| 15:1Δ10 | 10-Pentadecenoic | 7.926 | 7.715 | 1.7% |
| | acid | | | |
| 16:1Δ9 | Palmitoleic acid | 8.907 | 8.772 | 30.4% |
| 17:1Δ10 | 10-Heptadecenoic | 9.999 | 9.859 | 11.1% |
| | acid | | | |
| 18:1Δ6 | Petroselinic acid | 10.943 | — | 0.0% |
| 18:1Δ9 | Oleic acid | 10.978 | 10.862 | 33.7% |
| 18:1Δ11 | Vaccenic acid | 11.065 | 10.917 | 21.8% |
| 18:1Δ9, 12- | Ricinoleic acid | 12.737 | — | 0.0% |
| OH | | | | |
| 18:1Δ9, 12 | Linoleic acid | 11.656 | — | 0.0% |
| 19:1Δ7 | 7-Nondecenoic acid | 11.941 | — | 0.0% |
| 19:1Δ10 | 10-Nondecenoic acid | 12.01 | 11.888 | 6.1% |
| 20:1Δ5 | 5-Eicosenoic acid | 12.652 | — | 0.0% |
| 20:1Δ8 | 8-Eicosenoic acid | 12.713 | — | 0.0% |
| 20:1Δ11 | 11-Eicosenoic acid | 12.743 | 12.666 | 2.2% |
| 22:1Δ13 | Erucic acid | 13.406 | — | 0.0% |
| 24:1Δ15 | Nervonic acid | 13.86 | — | 0.0% |

### Example 5: tmsA Co-Factor Usage

The inventors performed the following experiments to determine which redox co-factor the tmsA enzyme (10-methylene reductase) uses to produce fully saturated 10-methyl fatty acids from the intermediate 10-methylene fatty acids.

Methods:

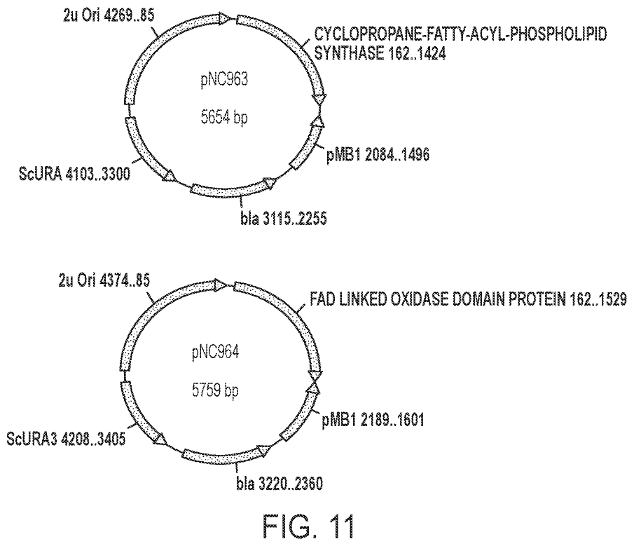

E. coli strains NS1161, NS1163, and NS1164 were used in this experiment; strain NS1161 was constructed by transforming the control (empty) vector plasmid pNC53 into E. coli CGSC 9407 (aka JW1653-1 Keio collection) which holds a kan R disruption of the native E. coli cyclopropane fatty acid synthase (cfa) gene. Strain NS1163 was constructed by transforming plasmid pNC963 (SEQ ID NO:95) (FIG. 11), containing the T. curvata tmsB gene under control of the constitutive tac promoter, into E. coli CGSC 9407. Strain NS1164 was constructed by transforming plasmid pNC964 (SEQ ID NO:96) (FIG. 11), containing the T. curvata tmsA gene under control of the constitutive tac promoter, into E. coli CGSC 9407.

Strain NS1163 was grown in 1 L LB media supplemented with 100 mg/L ampicillin for 24 hours at 37° C. (2×500 mL in 2 L baffled flasks). After cultivation, cells were harvested by centrifugation at 4000 rpm for 15 minutes in an Eppendorf 5810 R clinical centrifuge and washed twice in 100 mL PBS buffer. After concentration to 40 mL PBS buffer, cells were heat inactivated at 85° C. for 30 min. Inactivated cells were then dispensed into 1 mL aliquots and disrupted with 0.3 grams of 0.1 mm glass beads using a MP fastprep-24 on “E. coli” setting (MP biomedicals, LLC). Whole cell lysed suspension was collected by micro-centrifugation at 2000×g for 30 seconds to remove beads and then 0.7 mL of suspension per tube was transferred to new tubes and frozen at −80° C. until further use.

On the day of assay, strains NS1161 and NS1164 were grown via inoculation from overnight cultures (1:1000 dilution) in 50 mL LB medium supplemented with 100 mg/L ampicillin in 37° C. and 200 rpm in baffled shake flasks. After 4 hours of cultivation, cells were harvested at 5° C., washed 1× in ice cold PBS and then resuspended in 750 μL PBS in 1 mL plastic screw tubes. 0.3 grams of 0.1 mm glass beads were added and cells were lysed with a MP fastprep-24 on the “E. coli” setting. The cell suspension was then micro-centrifuged for 5 min at 12,000×g, and the supernatant transferred to a fresh tube and held on ice until assay.

Assay reaction: 700 μL of NS1163 whole lysate, 200 μL of 37.2 mg/mL NADPH solution (assay concentration 10 mM), 33.2 mg/mL NADH solution (assay concentration 10 mM), or PBS buffer, and 100 μL of cell free extract or PBS buffer. Assay tubes were sealed and rotated on a drum roller at 37° C. for 16 hours. To end the assay, tubes were frozen at −80° C., then lyophilized to dryness followed by in situ extraction and transesterification with methanolic HCL. Fatty acid profiles were determined by GC with flame ionization detection, and the 10-methyl fatty acid peak area was compared to the total fatty acid peak area to determine assay activity.

Results

Strain NS1163, which accumulates 10-methylene intermediate fatty acids via expression of the Thermomonospora curvata tmsB gene, was grown, harvested, inactivated, and lysed for use as a substrate for the tmsA (10-methylene reductase) assay. To this substrate cell-free extract E. coli strain NS1164 expressing the T. curvata tmsA gene or E. coli strain NS1161 containing an empty expression vector were added, along with NADPH or NADH. As observed Table 5 below, only the presence of T. curvata tmsA and NADPH resulted in synthesis of 10-methyl fatty acids in this assay.

TABLE 5

| E. coli (Δcfa | | relative | |
|---|---|---|---|
| background) cell free | co- | 10Me16 + 10Me18 | |
| extract | factor | peak area | SD |
| Tcu tmsA | NADPH | 0.059 | 0.003 |
| Tcu tmsA | NADH | ND | |
| Tcu tmsA | none | ND | |
| empty vector | NADPH | ND | |
| empty vector | NADH | ND | |
| empty vector | none | ND | |
| none | NADPH | ND | |
| none | NADH | ND | |
| none | none | ND | |

ND = Not detected by this assay

### Example 6: Expression of tmsB Genes in Yeast Yarrowia lipolytica and Arxula adeninivorans

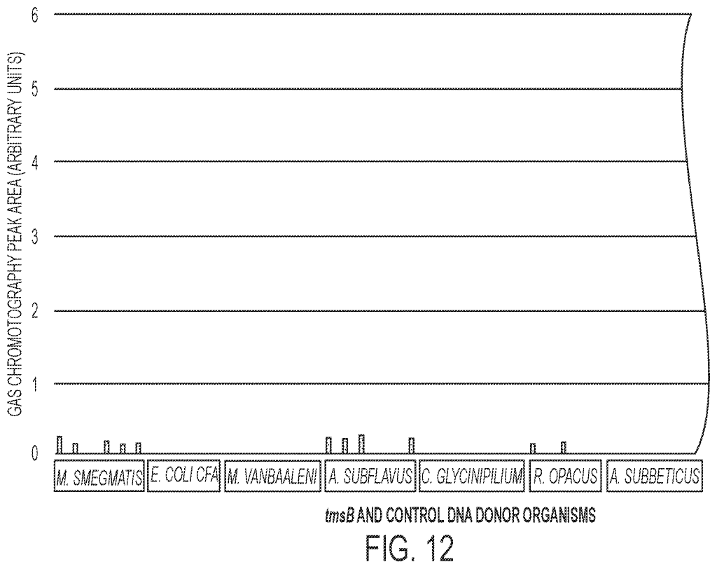

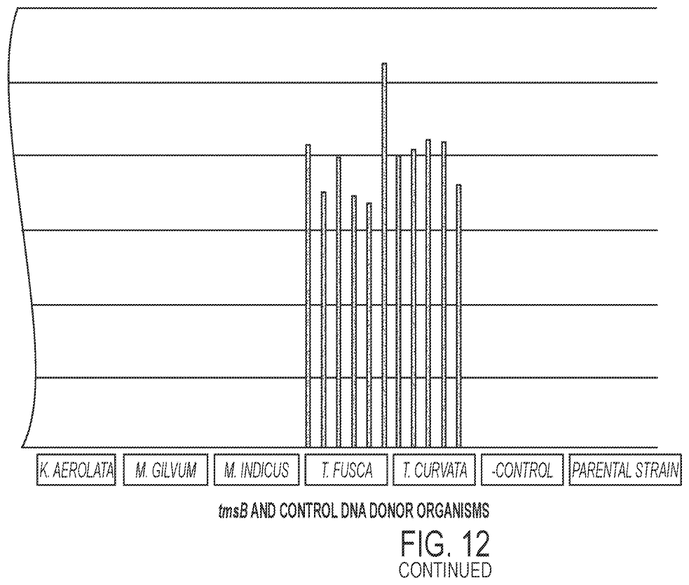

Sequences encoding the native bacterial codon tmsB sequences from Mycobacterium smegmatis, Mycobacterium vanbaaleni, Amycolicicoccus subflavus, Corynebacterium glyciniphilum, Rhodococcus opacus, Agromyces subbeticus, Knoellia aerolata, Mycobacterium gilvum, Mycobacterium sp. Indicus, Thermobifida fusca, and Thermomonospora curvata were cloned into a standard Yarrowia expression vector driven by the Y. lipolytica TEF1 promoter and containing an ARS68 Y. lipolytica replication origin, a nourseothricin antibiotic resistance gene for selection, and the 2μ origin and URA3 gene for high copy maintenance in Saccharomyces cerevisiae. Cloning was performed using the yeast-gap repair method (Shanks 2006) with selection on uracil dropout media. Y. lipolytica was transformed following a standard lithium acetate heat-shock protocol with selection on YPD medium supplemented with 500 μg/mL nourseothricin. Colonies were selected and transferred to a 96 well plate containing 300 μL nitrogen-limited lipid production media per well and incubated at 30° C. with shaking at 900 rpm for 96 hours. The medium contained 100 g/L glucose, 0.5 g/L urea, 1.5 g/L yeast extract, 0.85 g/L casamino acids, 1.7 g/L YNB base without amino acids, and 5.1 g/L potassium hydrogen phthalate at pH 5.5. After fermentation, cells were centrifuged, washed with distilled water, and frozen at −80° C. prior to lyophilization to dryness. Dried cells were transesterified in situ with 0.5 N HCl in methanol at 85° C. for 90 minutes to produce fatty acid methyl esters (FAME) suitable for gas chromatography analysis. These samples were dissolved in isooctane and injected into a gas chromatography system (Agilent Technologies) equipped with a flame ionization detector. Total C16 and C18 branched fatty acids were identified and quantified based on known standards and the 10 methylene and 10 methyl fatty acids identified in E. coli tins expression experiments. 10-methyl and 10-methylene fatty acid identities were verified by mass spec in an independent experiment. FIG. 12 shows that Y. lipolytica transformed with tmsB from T. fusca and T. curvata produced the highest amounts of 10-methylene stearic acid.

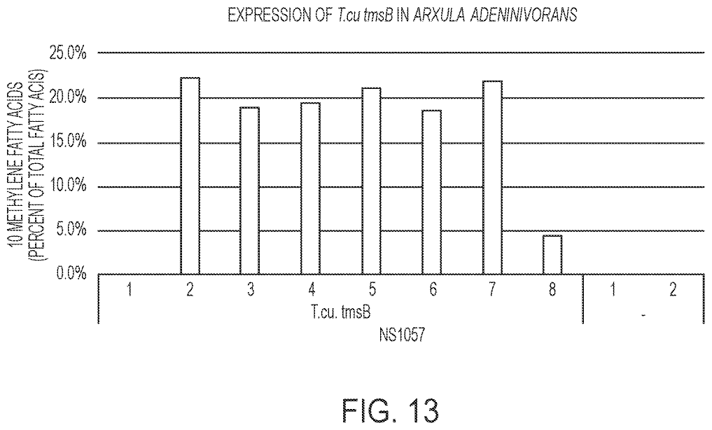

To test tmsB activity in Arxula adeninivorans, the top performing tmsB gene from Yarrowia, T. curvata tmsB (SEQ ID NO:75) was cloned into a constitutive expression vector under the Arxula ADH1 promoter, resulting in plasmid pNC1065. Individual transformant colonies were isolated and grown in a standard industrial media (with a high C:N ratio to promote lipid accumulation) for 4 days at 40° C. Cell pellets were isolated, washed once with water, and lyophilized. Total C16 and C18 fatty acids were transesterified as for Yarrowia strains and were analyzed by GC. FIG. 13 shows that A. adeninivorans transformed with tmsB from T. curvata produce 10-methylene fatty acids.

### Example 7: tmsA and tmsB Coexpression in Yarrowia lipolytica and Saccharomyces cerevisiae

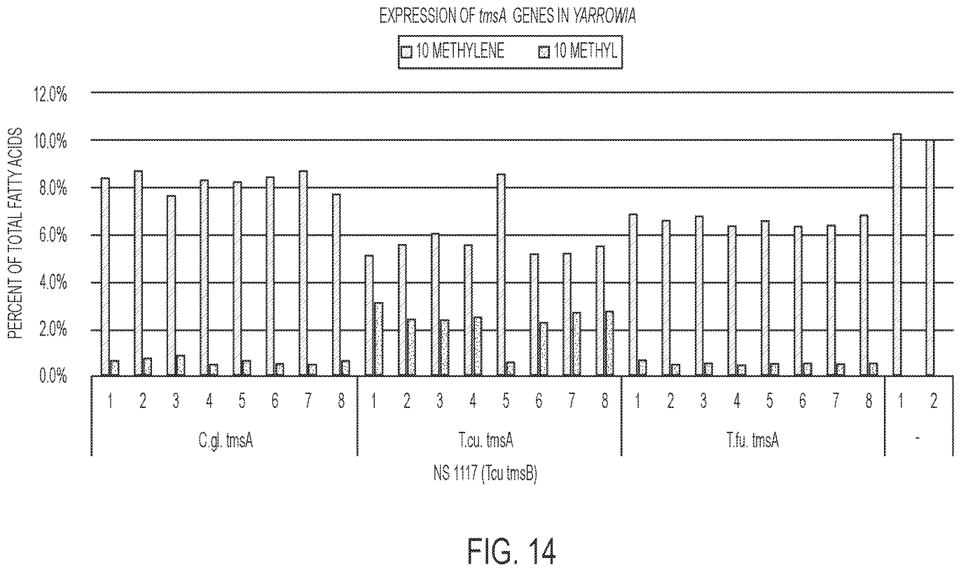

The inventors discovered that simultaneous expression of tmsA and tmsB genes can produce branched 10-methyl and 10-methylene fatty acids, respectively, in Saccharomyces and Yarrowia yeast strains. For expression in Yarrowia, plasmids constitutively expressing the native bacterial sequences for tmsA from T. curvata (pNC984), T. fusca (pNC983) and C. glutamicum (pNC991) were each transformed into strain NS1117 containing a stably integrated copy of the T. curvata tmsB gene (isolated from Example 6 above). Individual transformants were isolated and grown for 4 days at 30° C. in shake flask medium. Fatty acids were isolated and analyzed by GC as in Example 6. As shown in FIG. 14, all tmsA genes analyzed produce at detectible levels of 10 methyl fatty acids in Yarrowia, compared to the parental strain. The T. curvata tmsA gene produced more 10-methyl fatty acids than the other tmsA genes analyzed.

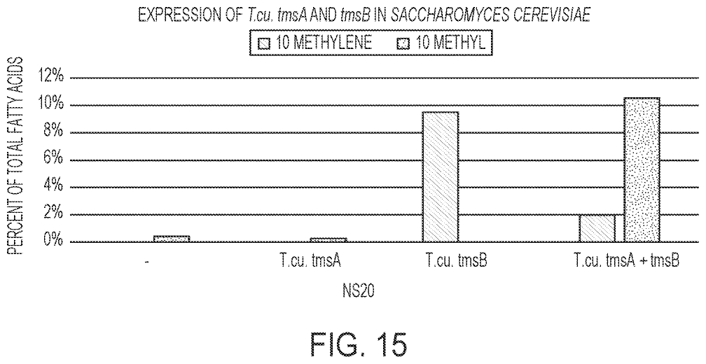

For expression in Saccharomyces, plasmids with demonstrated gene activity in Yarrowia, pNC984 (T. curvata tmsA with a NAT marker) and pNC1025 (T. curvata tmsB with a HYG marker) were transformed individually and together into S. cerevisiae strain NS20, and transformants were selected on media containing the appropriate antibiotic(s). Individual transformation isolates were grown for 2 days in YPD medium at 30° C. Cell pellets were processed, and total fatty acids were analyzed as for Yarrowia. As shown in FIG. 15, the strain transformed with only tmsB produced only 10-methylene fatty acids, and the strain transformed with both tmsA and tmsB produced a relatively high percentage of 10-methyl fatty acids.

### Example 8: Expression of a tmsA-B Fusion Protein in E. coli, Saccharomyces Ceverisiae, Yarrowia lipolytica and Arxula adeninivorans

The inventors discovered that expressing the tmsA and tmsB enzymes in a single polypeptide improves conversion of 10-methylene fatty acids to 10-methyl fatty acids. Single proteins containing both tmsA and tmsB activity were created by fusing the genes for Thermomonospora curvata tmsA and tmsB in frame, separated by a flexible linker domain. The Thermomonospora curvata tmsA and tmsB enzymes were chosen because they produced the most 10-methyl branched fatty acids in yeast. A short 12 amino acid linker with the sequence AGGAEGGNGGGA (SEQ ID NO: 99) which occurs naturally in the Yarrowia FAS2 gene was chosen to connect the two enzymes. Two fusion enzymes were tested for activity in bacteria and yeast, tmsA-B (NG540; encoded by SEQ ID NO:97) and tmsB-A (NG541; encoded by SEQ ID NO:98).

For E. coli expression, plasmids pNC1069 and pNC1070 containing the T. curvata tmsA-B and tmsB-A genes with the tac promoter and trpT′ terminator were each transformed into E. coli CGSC 9407. Individual transformed strains were grown and total fatty acids were assayed as in Example 2 above. As shown in Table 6 below, both the tmsA-B and tmsB-A genes resulted in production of methylated stearic acid in E. coli.

TABLE 6
Methylation of oleic and vaccenic acid was calculated as the percent
of C18:1 fatty acids converted into 10- and 12-methyl fatty acids.

| Vector | % C18:1 methylated |
|---|---|
| None | 0 |
| T. curvata tmsA-B | 19.4 |
| T. curvata tmsB-A | 26.25 |

For Saccharomyces cerevisiae and Yarrowia lipolytica expression, NG540 (SEQ ID NO:97) and NG541 (SEQ ID NO:98) were individually cloned into standard Yarrowia expression vectors containing a yeast 2u origin of replication for high copy retention in Saccharomyces, resulting in the respective vectors pNC1067 and pNC1068.

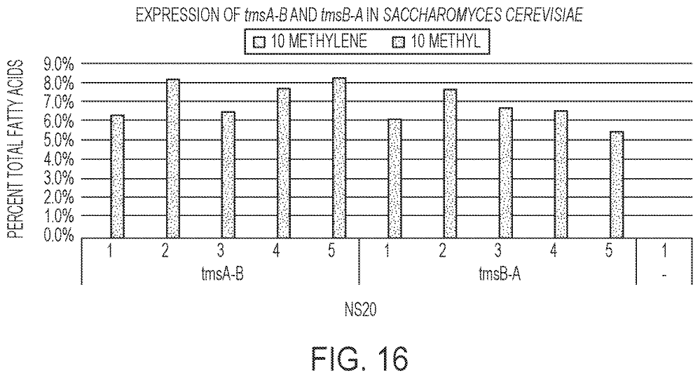

Plasmids pNC1067 and pNC1068 were transformed into Saccharomyces strain NS20 by a standard protocol and individual transformed strains were selected for assay of branched fatty acid production. Strains were grown for 2 days at 30° C. in 25 ml YPD medium. Cell pellets were lyophilized and total fatty acids were analyzed by basic transesterification and GC analysis as in Example 2. FIG. 16 shows that expression of both tmsA-B and tmsB-A in S. cerevisiae led to production of 10 methyl fatty acids.

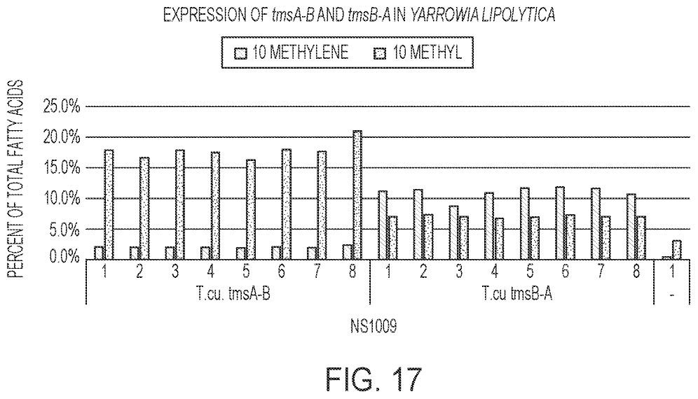

Plasmids pNC1067 and pNC1068 were transformed into Yarrowia lipolytica by a standard heat shock protocol. Individual resulting transformant strains were chosen for analysis of 10-methylene and 10-methyl fatty acid production. Strains were grown and analyzed by GC as in Example 7. FIG. 17 shows that expression of both tmsA-B and tmsB-A in Y. lipolytica led to production of 10 methyl fatty acids, although tmsA-B was more efficient at converting 10-methylene fatty acids to 10-methyl fatty acids.

For expression in Arxula adeninivorans, NG540 was cloned into a standard expression vector containing the constitutive Arxula ADH1 promoter resulting in pNC1151. pNC1151 was transformed into Arxula strain NS1166 and individual transformants were selected to assay of 10-methyl fatty acid production. Arxula strains were grown and analyzed by GC as in Example 7.

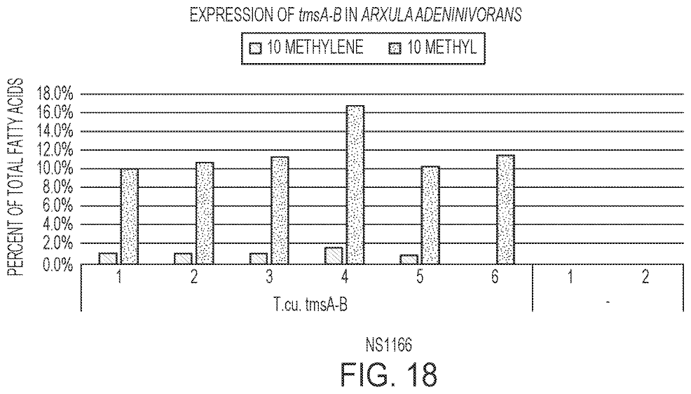

These experiments showed that 10-methyl C16 and C18 fatty acids were detected in E. coli. (Table 6), Saccharomyces cerevisiae (FIG. 16), Yarrowia lipolytica (FIG. 17), and Arxula adeninivorans (FIG. 18), indicating that the fusion enzymes contain both tmsA and tmsB activities. The low production of 10-methylene intermediates (undetectable in E. coli and Saccharomyces, at low levels in Yarrowia and Arxula) indicate that the fusion protein efficiently converts unsaturated fatty acids into 10 methyl fatty acids.

### Example 9: tmsB Sequence Analysis

TmsB protein sequences coded by the tmsB genes from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, Thermobifida fusca, and Thermomonospora curvata were aligned with the cyclopropane fatty acid synthase (Cfa) enzyme from Escherichia coli with the CLUSTAL OMEGA software program (European Molecular Biology Laboratory, EMBL). FIGS. 19A-D show the alignment of these protein sequences. E. coli Cfa shares homology to the TmsB enzyme and carries out a similar reaction to TmsB, with methylation of a fatty acid phospholipid double bond, but produces a cyclopropane moiety rather than a methylene moiety.

Certain amino acids of the E. coli Cfa enzyme are thought to bind the active site bicarbonate ion. Iwig et al., J. Am. Chem. Soc. 127:11612-13(2005). These amino acids are C139, E239, H266, 1268, and Y317 of the E. coli enzyme, which are conserved in the consensus tmsB protein sequence (C160, E266, H293, 1295, and Y348 on the T. curvata TmsB sequence SEQ ID NO:76).

Additionally, there are sixteen amino acid residues that are conserved for all twelve TmsB protein sequences, but not in the E. coli Cfa sequence. These amino acids may be specific for 10-methylene addition to fatty acid phospholipids rather than the cyclopropane addition performed by the E. coli Cfa protein. These conserved amino acids, numbered with the T. curvata TmsB sequence, are D23, G24, A59, H128, F147, Y148, L180, L193, M203, G236, A241, R313, R318, E320, L359, L400 of SEQ ID NO:76.

A BLASTp conserved domains analysis (National Center for Biotechnology Information, NCBI) identifies a S-adenosylmethionine-dependent methyltransferase domain from amino acids 192-291 of T. curvata TmsB. S-adenosylmethionine binding site amino acid residues are identified as V196, G197, C198, G199, W200, G201, G202, T219, L220, Q246, D247, Y248, and D262.

Table 7 shows the percent sequence identity of the indicated protein relative to T. curvata tmsB:

TABLE 7

| Species | % Identity |
|---|---|
| Thermomonospora curvata tmsB | 100 |
| Mycobacterium smegmatis tmsB | 60 |
| Mycobacterium vanbaaleni tmsB | 59 |
| Amycolicicoccus subflavus tmsB | 55 |
| Corynebacterium glyciniphilum tmsB | 47 |
| Corynebacterium glutamicum tmsB | 50 |
| Rhodococcus opacus tmsB | 59 |
| Agromyces subbeticus tmsB | 57 |
| Knoellia aerolata tmsB | 47 |
| Mycobacterium gilvum tmsB | 58 |
| Mycobacterium sp. Indicus tmsB | 58 |
| Thermobifida fusca tmsB | 67 |
| Escherichia coli Cfa | 34 |

As shown in Table 7, there is a great deal of variation among the tmsB protein sequences from the different species. Nevertheless, despite the sequence variation, several of the proteins are shown herein to have the same ability to catalyze the production of a methylene-substituted lipid.

### Example 10: tmsA Sequence Analysis

TmsA protein sequences coded by the tmsA genes from Mycobacterium smegmatis, Mycobacterium vanbaaleni, Amycolicicoccus subflavus, Corynebacterium glyciniphilum, Corynebacterium glutamicum, Rhodococcus opacus, Agromyces subbeticus, Knoellia aerolata, Mycobacterium gilvum, Mycobacterium sp. Indicus, Thermobifida fusca, and Thermomonospora curvata were aligned with the Glycolate oxidase subunit GlcD enzyme from Escherichia coli with the CLUSTAL OMEGA software program (European Molecular Biology Laboratory, EMBL). The E. coli GlcD enzyme does not appear to perform a similar enzymatic reaction as TmsA, but it is the most closely homologous protein to TmsA in the E. coli genome.

FIGS. 20A-E show the alignment of the TmsA proteins. There are 114 amino acid residues that are conserved for all twelve TmsA protein sequences, but not in the E. coli GlcD sequence. These amino acids are (numbered according to the T. curvata sequence (SEQ ID NO:74)): R31, A33, S37, N38, L39, F40, R43, D52, V59, D63, G73, M74, T76, Y77, D79, L80, V81, L85, P91, V93, V94, Q96, L97, T99, I100, T101, A105, G108, G110, E112, 5113, 5115, F116, R117, N118, P121, H122, E123, V125, E127, G133, P154, N155, Y157, Y162, L166, E171, V173, V177, H181, V208, G213, F216, Y222, L223, 5236, D237, Y238, T239, Y245, 5247, D254, T257, Y261, W263, R264, W265, D266, D268, W269, C272, A275, G277, Q279, R284, W287, R293, 5294, G318, E232, V325, P328, E330, F339, F343, W353, C355, P356, W363, L365, Y366, P367, N376, F379, W380, V383, P384, N395, E399, G407, H408, K409, 5410, L411, Y412, 5413, Y417, F422, Y426, G428, R443, L447, and V452.

A BLASTp conserved domains analysis (National Center for Biotechnology Information, NCBI) identifies a Flavin adenine dinucleotide (FAD) binding domain from amino acids 9-141 of T. curvata TmsA (SEQ ID NO:74), as well as a FAD/FMN-containing dehydrogenase domain from amino acids 22-444. Table 8 shows the percent sequence identity of the indicated protein relative to T. curvata tmsA:

TABLE 8

| Species | % Identity |
|---|---|
| Thermomonospora curvata tmsA | 100 |
| Mycobacterium smegmatis tmsA | 61 |
| Mycobacterium vanbaaleni tmsA | 61 |
| Amycolicicoccus subflavus tmsA | 60 |
| Corynebacterium glyciniphilum tmsA | 55 |
| Corynebacterium glutamicum tmsA | 53 |
| Rhodococcus opacus tmsA | 61 |
| Agromyces subbeticus tmsA | 59 |
| Knoellia aerolata tmsA | 60 |
| Mycobacterium gilvum tmsA | 59 |
| Mycobacterium sp. Indicus tmsA | 58 |
| Thermobifida fusca tmsA | 64 |
| Escherichia coli GlcD | 28 |

As shown in Table 8, there is a great deal of variation among the tmsA protein sequences from the different species. Nevertheless, despite the sequence variation, several of the proteins are shown herein to have the same ability to catalyze the production of a methyl-substituted lipid.

### INCORPORATION BY REFERENCE

Each of the patents, published patent applications, and non-patent references cited herein is hereby incorporated by reference in its entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A yeast cell comprising a methyltransferase protein encoded by a methyltransferase gene and either a branched (methyl)lipid or an exomethylene-substituted lipid, wherein:
the methyltransferase protein comprises an amino acid sequence with at least 90% sequence identity with the amino acid sequence of SEQ ID NO:76;
the branched (methyl)lipid or exomethylene-substituted lipid is a carboxylic acid, carboxylate, ester, thioester, or amide; and
the branched (methyl)lipid comprises a saturated or unsaturated branched aliphatic chain comprising a branching methyl group or the exomethylene-substituted lipid comprises a branched aliphatic chain that is branched because the aliphatic chain is substituted with an exomethylene group.

2. The yeast cell of claim 1, wherein the branched (methyl)lipid or the exomethylene-substituted lipid comprises a linear lipid with a chain length of 14-20 carbons and a methyl branch at the Δ9, Δ10, or Δ11 position.

3. The yeast cell of claim 2, wherein the branched (methyl)lipid or the exomethylene-substituted lipid is a diacylglycerol, triacylglycerol, or phospholipid, and the diacylglycerol, triacylglycerol, or phospholipid comprises an ester of 10-methylstearate or an ester of 10-methylenestearate.

4-6. (canceled)

7. The yeast cell of claim 1, wherein at least 1% by weight of the fatty acids of the yeast cell are one or more linear fatty acids with a chain length of 14-20 carbons and a methyl branch at the Δ9, Δ10, or Δ11 position.

8. The yeast cell of claim 1, wherein the yeast cell comprises at least 1% lipid as measured by % dry cell weight.

9-11. (canceled)

12. The yeast cell of claim 1, further comprising a recombinant reductase gene.

13. The yeast cell of claim 12, wherein the recombinant reductase gene encodes tmsA from Thermomonospora curvata.

14. The yeast cell of claim 1, wherein:
the methyltransferase gene is codon-optimized for the yeast cell; or
the yeast cell further comprises a reductase gene and the reductase gene is codon-optimized for the yeast cell.

15-19. (canceled)

20. The yeast cell of claim 1, wherein the yeast cell is selected from the group consisting of Arxula, Saccharomyces, and Yarrowia.

21. The yeast cell of claim 20, wherein the yeast cell is selected from the group consisting of Arxula adeninivorans, Saccharomyces cerevisiae, and Yarrowia lipolytica.

22-23. (canceled)

24. The yeast cell of claim 1, wherein the methyltransferase gene comprises either:
a nucleotide sequence with at least about 95% sequence identity with the nucleotide sequence of SEQ ID NO:75; or
the nucleotide sequence of SEQ ID NO:75.

25-28. (canceled)

29. A method of producing a branched (methyl)lipid or exomethylene-substituted lipid, comprising contacting the yeast cell of claim 1 with oleic acid, methionine, or both oleic acid and methionine.

30-49. (canceled)

50. The yeast cell of claim 12, wherein the reductase protein is in a fusion protein with the tmsB enzyme.


SEQUENCE LISTING (101 sequences)

SEQ ID NO: 1 - DNA, 1401 bp, Mycobacterium smegmatis
  source 1..1401: mol_type = genomic DNA
gtgtctgtggttactactgacgcacaggctgcccatgccgccggcgtctcgcgtcttctggccagctaccgggcgatcccgcccagcgcgacagtgcgccttgcgaaaccgacgtccaacctgttccgcgcccgcgcccgcaccaatgtgaagggtctcgacgtctcgggcctgaccggtgtgatcggtgtcgacccggacgcgcgcaccgccgatgtggcgggcatgtgcacctacgaggacctggtggcggccacgcttccgtacggccttgccccactggtggtgccgcagctcaagaccatcacgctcggtggcgcggtcaccggtctgggcatcgagtccacgtcgttccgcaacggtctgccgcacgaaagtgtcctggagatggacatcttgaccggttcgggcgagatcgtcacggcctcaccggatcagcactcggatctgttccatgcgttccccaattcatatggaacccttggttattccacccggctgcgcatcgaactggagcccgtgcacccgtttgtggcgttgcgccacctgcgctttcactcgatcaccgatctggtcgcggcgatggaccggatcatcgagaccggcgggctggacggtgaacccgtcgactacctcgacggcgtggtgttcagcgcgactgagagttacctgtgtgttggcttcaagacgaaaacgccggggccggtcagcgattacacaggtcagcagatcttctaccggtcgatccagcatgacggcgacaccggcgccgagaaacacgaccggctgaccatccacgactacctgtggcgctgggacaccgactggttctggtgctcacgggcattcggcgctcagcatccggtgatccgcaggttctggccgcggcggctgcgccgcagcagcttctactggaagctggtggcctacgaccagcggtacgacatcgccgaccgtatcgagaagcgcaacgggcgcccgccgcgcgagcgggtggtccaggacgtcgaggtgcccatcgagcggtgcgcggacttcgtcgagtggttcctgcagaatgtgccgatcgagccgatctggctgtgccccctacggttgcgtgacagcgccgacggcggtgcctcgtggcccctgtatccgctgaaggcgcaccacacctacgtcaacatcggtttctggtcatcagtgccggtgggccccgaggagggccacaccaaccgcctcatcgagaaaaaagtcgcggagctggacgggcacaaatctttgtactcggacgcttattacacacgtgacgaattcgacgagctgtacggcggtgaggtctacaacaccgtcaagaagacgtacgacccggattcacgtctgctagacctgtattcgaaggcggtgcaaagacaatga

SEQ ID NO: 2 - protein, 466 aa, Mycobacterium smegmatis
VSVVTTDAQAAHAAGVSRLLASYRAIPPSATVRLAKPTSNLFRARARTNVKGLDVSGLTGVIGVDPDARTADVAGMCTYEDLVAATLPYGLAPLVVPQLKTITLGGAVTGLGIESTSFRNGLPHESVLEMDILTGSGEIVTASPDQHSDLFHAFPNSYGTLGYSTRLRIELEPVHPFVALRHLRFHSITDLVAAMDRIIETGGLDGEPVDYLDGVVFSATESYLCVGFKTKTPGPVSDYTGQQIFYRSIQHDGDTGAEKHDRLTIHDYLWRWDTDWFWCSRAFGAQHPVIRRFWPRRLRRSSFYWKLVAYDQRYDIADRIEKRNGRPPRERVVQDVEVPIERCADFVEWFLQNVPIEPIWLCPLRLRDSADGGASWPLYPLKAHHTYVNIGFWSSVPVGPEEGHTNRLIEKKVAELDGHKSLYSDAYYTRDEFDELYGGEVYNTVKKTYDPDSRLLDLYSKAVQRQ

SEQ ID NO: 3 - DNA, 1314 bp, Mycobacterium smegmatis
  source 1..1314: mol_type = genomic DNA
atgaccacattcaaagaacgcgagacgtccacagcggaccgcaagctcaccctggccgagatcctcgagatcttcgccgcgggtaaggagccgctgaagttcactgcgtacgacggcagctcggccggtcccgaggacgccacgatgggtctggacctcaagaccccgcgtgggaccacctatctggccacggcacccggcgatctgggcctggcccgtgcgtatgtctccggtgacctggagccgcacggcgtgcatcccggcgatccctacccgctgctgcgcgccctggccgaacgcatggagttcaagcgcccgcctgcgcgtgtgctggcgaacatcgtgcgctccatcggcatcgagcacctcaagccgatcgcaccgccgccgcaggaggcgctgccccggtggcgccgcatcatggagggcctgcggcacagcaagacccgcgacgccgaggccatccaccaccactacgacgtgtcgaacacgttctacgagtgggtgctgggcccgtcgatgacctacacgtgcgcgtgctaccccaccgaggacgcgaccctcgaagaggcccaggacaacaagtaccgcctggtgttcgagaagctgcgcctgaagcccggtgaccggttgctcgacgtgggctgcggctggggcggcatggtccgctacgcggcccgccacggcgtcaaggcgctcggtgtcacgctcagccgcgaacaggcgacgtgggcgcagaaggccatcgcccaggaaggtctcaccgatctggccgaggtgcgtcacggtgattaccgcgacgtcatcgaatccgggttcgacgcggtgtcctcgatcgggctgaccgagcacatcggcgtgcacaactacccggcgtacttcaacttcctcaagtcgaagctgcgcaccggtggcctgctgctcaaccactgcatcacccgcccggacaaccggtcggcgccatcggccggcgggttcatcgacaggtacgtgttccccgacggggagctcaccggctcgggccgcatcatcaccgaggcccaggacgtgggccttgaggtgatccacgaggagaacctacgcaatcactatgcgatgacgctgcgcgactggtgccgcaacctggtcgagcactgggacgaggcggtcgaagaggtcgggctgcccaccgcgaaggtgtggggcctgtacatggccggctcacgtctgggcttcgagaccaatgtggttcagctgcaccaggttctggcggtcaagcttgacgatcagggcaaggacggcggactgccgttgcggccctggtggtccgcctag

SEQ ID NO: 4 - protein, 437 aa, Mycobacterium smegmatis
MTTFKERETSTADRKLTLAEILEIFAAGKEPLKFTAYDGSSAGPEDATMGLDLKTPRGTTYLATAPGDLGLARAYVSGDLEPHGVHPGDPYPLLRALAERMEFKRPPARVLANIVRSIGIEHLKPIAPPPQEALPRWRRIMEGLRHSKTRDAEAIHHHYDVSNTFYEWVLGPSMTYTCACYPTEDATLEEAQDNKYRLVFEKLRLKPGDRLLDVGCGWGGMVRYAARHGVKALGVTLSREQATWAQKAIAQEGLTDLAEVRHGDYRDVIESGFDAVSSIGLTEHIGVHNYPAYFNFLKSKLRTGGLLLNHCITRPDNRSAPSAGGFIDRYVFPDGELTGSGRIITEAQDVGLEVIHEENLRNHYAMTLRDWCRNLVEHWDEAVEEVGLPTAKVWGLYMAGSRLGFETNVVQLHQVLAVKLDDQGKDGGLPLRPWWSA

SEQ ID NO: 5 - DNA, 1380 bp, Agromyces subbeticus
  source 1..1380: mol_type = genomic DNA
gtgtccgctcctgcgaccgatgcacgaaccgcccacgccgacggcgtggagcgattgctcgagagttatcgggcggtgccggcggccgcatcggtgcggctcgccaagcgcacctcgaacctcttccggtcccgagcggcgacggatgcccctggcctcgacacctccggcctgacccacgtcatcgcggtcgaccccggggcgcgcacggccgacgtcgccggcatgtgcacctacgacgacctcgtcgccgcgacactgccgcatgggctcgcgccactcgtggtgccgcaactgaagaccatcaccctcgggggcgccgtaacgggactcggcatcgagtcgacgtcgttccgcaacggtctgccgcacgagtcggtgctcgagatcgacgtgctcaccggcgcaggcgagatcatcacggcgtcgccgatcgagcacgcagagctgttccgcgccttccccaactcgtacggcaccctcggctacgccgtgcgcctgcgcatcgagctcgagccggtcgagccgttcgtcgcactcacgcaccttcggttccatgcgctcaccgacctcatcgaggcaatggagcgcatcatcgagaccggtcgactcgacggggttgccgtcgattccctcgacggcgtggtgttcagcgctgaagagagctacctgtgcgtcggcacgcagaccgcggcatccggcccggtcagcgactacacccgccagcagatcttctatcgctccatccagcatgacgacggtgcgaagcacgaccggctcaccatgcacgactacctgtggcgctgggacgccgactggttctggtgctcgcaggcgttcggcgcgcagcatccgctgattcgccggttctggccgcggcgataccggcgcagccgctcgtactcgacgctcatgcgcctcgaacggcgattcgacctcggcgatcgcctcgagaagctcaagggccggccggcgcgcgaacgcgtgatccaagacgtcgaggtgccgatcgggcgcaccgtcggcttcctcgaatggttcctcgcgaacgtgccgatcgagccgatctggttgtgcccgctgcgcctgcggggcgaccgcggctggcctctctacccgatccggccgcagcagacctacgtcaacatcggcttctggtcgacggttccggtgggcggctccgagggcgagacgaaccgctcgatcgagcgcgccgtgagcgagttcgacggacacaagtcgctgtactccgactcgtactactcgcgcgaggagttcgaggagctctacggcggcgaggcgtaccgggccgtgaagcggcgatacgaccccgactctcgactgctcgacctctatgcgaaggcggtgcaacggcgatga

SEQ ID NO: 6 - protein, 459 aa, Agromyces subbeticus
VSAPATDARTAHADGVERLLESYRAVPAAASVRLAKRTSNLFRSRAATDAPGLDTSGLTHVIAVDPGARTADVAGMCTYDDLVAATLPHGLAPLVVPQLKTITLGGAVTGLGIESTSFRNGLPHESVLEIDVLTGAGEIITASPIEHAELFRAFPNSYGTLGYAVRLRIELEPVEPFVALTHLRFHALTDLIEAMERIIETGRLDGVAVDSLDGVVFSAEESYLCVGTQTAASGPVSDYTRQQIFYRSIQHDDGAKHDRLTMHDYLWRWDADWFWCSQAFGAQHPLIRRFWPRRYRRSRSYSTLMRLERRFDLGDRLEKLKGRPARERVIQDVEVPIGRTVGFLEWFLANVPIEPIWLCPLRLRGDRGWPLYPIRPQQTYVNIGFWSTVPVGGSEGETNRSIERAVSEFDGHKSLYSDSYYSREEFEELYGGEAYRAVKRRYDPDSRLLDLYAKAVQRR

SEQ ID NO: 7 - DNA, 1254 bp, Agromyces subbeticus
  source 1..1254: mol_type = genomic DNA
atcctcgagatcgtcgtcgccggtcggctgccgctgaggttcaccgcctacgacgggagctcggcggggccgcctgacgccctgttcggcctcgacctgaagactccgcgaggaacgacctatctcgccaccggccgcggcgatctcggcctcgcccgcgcctacatcgcgggcgacctcgagatacagggggtgcaccccggagacccctacgagctgctcaaggcactcgccgacagcctggtcttcaagctgccaccgccgcgggtgatgacccagatcatccgttcgatcggcgtcgaacatctgcggccgatcgcgccgccgccgcaagaggtgccgccccggtggcgccgcatcgccgaggggctccgacacagcaagggccgcgacgccgaagcgatccaccaccactacgacgtgtcgaacaccttctacgaatgggtgctcgggccgtcgatgacctacacgtgcgcgtgctacccgggcctcgacgcatccctcgacgaggcgcagcagaacaagtaccggctcgtgttcgagaagctgcggctgaagccgggcgaccgactgctcgacgtcggctgcgggtggggcggcatggtgcgctacgccgcgcgccacggcgtgcaggcgttgggcgtgaccctgtcgcgagagcagacggcgtgggcgcagcaggcgatcgccgtcgagggcctcgccgacctcgccgaggtgcgctacggcgactaccgcgacatcgccgaagacggcttcgatgcggtgtcatcgatcgggctgctcgagcacatcggcgtgcgcaactacgcttcgtatttcggctttctgcagtcgcgcttgcggcccgggggactcttgctcaaccactgcatcacccggcccgacaatcgctccgagccgtcggcgcgcggcttcatcgaccggtacgtgttccccgacggagagctcaccggctcgggccgcatcatcaccgaggcgcaggatgtcggcttcgaagtgctgcacgaagagaacctgcgtcagcattatgcactgacactgcgcgattggtgcgccaacctcgtcgcgcactgggaagaggcggtcgccgaggtcgggctgccgaccgcgaaggtgtggggcctctacatggccgggtcacggctcgcgttcgagagcggcggcatccagttgcaccaggtgctggcggtcagaccagacgatcgcagcgacgccgcccagctgccgctgcggccgtggtggacgccatag

SEQ ID NO: 8 - protein, 417 aa, Agromyces subbeticus
MLEIVVAGRLPLRFTAYDGSSAGPPDALFGLDLKTPRGTTYLATGRGDLGLARAYIAGDLEIQGVHPGDPYELLKALADSLVFKLPPPRVMTQIIRSIGVEHLRPIAPPPQEVPPRWRRIAEGLRHSKGRDAEAIHHHYDVSNTFYEWVLGPSMTYTCACYPGLDASLDEAQQNKYRLVFEKLRLKPGDRLLDVGCGWGGMVRYAARHGVQALGVTLSREQTAWAQQAIAVEGLADLAEVRYGDYRDIAEDGFDAVSSIGLLEHIGVRNYASYFGFLQSRLRPGGLLLNHCITRPDNRSEPSARGFIDRYVFPDGELTGSGRIITEAQDVGFEVLHEENLRQHYALTLRDWCANLVAHWEEAVAEVGLPTAKVWGLYMAGSRLAFESGGIQLHQVLAVRPDDRSDAAQLPLRPWWTP

SEQ ID NO: 9 - DNA, 1428 bp, Amycolicicoccus subflavus
  source 1..1428: mol_type = genomic DNA
atgacgcctgaagctagtgcggcggcgcacgccgctgcggtggatcgcctcatccatagctatcgggcgattcctgatgacgcgccggtgcggctggcgaagaagacgtcaaacctattccgccacagggaaaagacttctgctcctgggcttgacgtatccggcctggctcgcgtgattgggatcgactcagacactcgcactgccgacgttggcggcatgtgcacatacgaggaccttgtcgcggcgacgctcgaatacgatctggtccccctggtcgtcccgcaactcaaaacgatcactctcggcggcgcggtgacgggcctgggaattgagtccacctcgttccgcaatgggcttccccatgaatctgttctcgaaatggatatcctgacgggcgccggggaggtcgtcacggccggcccggaaggcccccatagcgatttgtactgggggtttccgaattcgtacggcacgctcggctatgcgacgcgcctgcgcatcgaactagaaccggtcgagccgtacgtcgaactcaggcacctgcggttcactagcctcgatgagcttcaggagacacttgacaccgtttcgtacgaacacacgtatgacggggaacccgttcattacgtcgatggagtcatgttctcagccacggaaagctacctcacgcttggccgtcagacgagcgaacccggcccggtcagcgactacaccggaaaccagatctactaccgttcaatacagcacggtggcgctgaaactcccgtcgtcgaccggatgaccattcatgactatctatggcgctgggatactgactggttctggtgctcgcgtgccttcggaacgcaacacccagtggtccggagattctggccacgccgctatcgccgcagcagcttctactggaagctgatcgcgcttgaccgccaggttgggctcgcggacttcatcgaacaacggaagggcaacctcccccgggaacgcgtagtccaggacatcgaggtcccgatcgagaacactgcgagcttcttgcggtggttcttggcgaacgtgccgatcgagccggtatggctatgcccgctgcgcctgcgaaaaacacgcagccccggcctgccttcgccgacgtccccggcttcacgcccatggcccctctatccgctcgagcctcagcgcacatacgtcaatgttggcttctggtcagcggtgccggtcgtggccggccagcccgaggggcacaccaaccggatgatcgagaacgaagtcgatcgccttgacggtcacaaatcgctgtactcagatgcgttttacgagcgaaaagagtttgacgcgctgtacggcggcgatacctatagagaactcaaagagacctacgacccaaacagccggttacttgatctctatgcaaaggcggtgcaaggacgatga

SEQ ID NO: 10 - protein, 475 aa, Amycolicicoccus subflavus
MTPEASAAAHAAAVDRLIHSYRAIPDDAPVRLAKKTSNLFRHREKTSAPGLDVSGLARVIGIDSDTRTADVGGMCTYEDLVAATLEYDLVPLVVPQLKTITLGGAVTGLGIESTSFRNGLPHESVLEMDILTGAGEVVTAGPEGPHSDLYWGFPNSYGTLGYATRLRIELEPVEPYVELRHLRFTSLDELQETLDTVSYEHTYDGEPVHYVDGVMFSATESYLTLGRQTSEPGPVSDYTGNQIYYRSIQHGGAETPVVDRMTIHDYLWRWDTDWFWCSRAFGTQHPVVRRFWPRRYRRSSFYWKLIALDRQVGLADFIEQRKGNLPRERVVQDIEVPIENTASFLRWFLANVPIEPVWLCPLRLRKTRSPGLPSPTSPASRPWPLYPLEPQRTYVNVGFWSAVPVVAGQPEGHTNRMIENEVDRLDGHKSLYSDAFYERKEFDALYGGDTYRELKETYDPNSRLLDLYAKAVQGR

SEQ ID NO: 11 - DNA, 1311 bp, Amycolicicoccus subflavus
  source 1..1311: mol_type = genomic DNA
atgaaggcagtgttgacggcgtttacggctccccaactcgaaaggatgaacgtcgctgagatactcagcgcggtactcgggcgagatttcccgatccggttcactgcgtacgacggcagcgcgctcggccccgaaaccgcccgctacggcttgcacctcacgacgccgcgcgggctgacctacctcgctaccgcgcccggtgatctcgggctcgcacgcgcgtacgtgtccggcgacctcgaggtcagtggggttcatcagggtgacccgtacgagataatgaagatcctcgcgcatgacgtccgggtgcggcggccctcgccagcaacgatcgcttcgatcatgcggtccctcggctgggaacgcttgcgaccggtcgcgccgcccccgcaagagaacatgccccgttggcgccggatggcccttggcctgctgcactcgaagagccgtgatgctgcggcaatccaccatcattacgacgtgtcgaacgagttttacgagcacatcctcggcccgtcgatgacgtacacatgcgcggcctaccccagcgcagacagttccctggaggaagcacaggacaacaagtaccgactcgtcttcgagaaacttggcctgaaagccggggatcgcctgcttgacgtcgggtgcgggtggggcggcatggtgcggttcgccgctaagcgcggcgttcatgtcatcggtgcgacattgtcccgcaaacaggcggaatgggctcagaagatgattgcccatgaaggattgggcgatctggcggaagtccgtttctgcgactaccgcgatgtcacagaggcgggcttcgacgcagtgtcgtcgatcggcctcactgaacacatcggtttggcgaactacccgtcgtacttcggcttcctgaaggacaagttgcggccaggcggacgactgctgaaccattgcatcactcgcccgaacaaccttcaaagcaaccgcgcaggtgacttcattgaccggtacgttttccctgacggagagctcgccggacctggcttcatcatttcagctgtccacgacgccggtttcgaggtgcggcacgaagagaacctccgcgagcactacgcactgacgctgcgggactggaaccgcaacctcgctcgcgactgggacgcgtgtgtgcacgcctccgacgagggcaccgcccgcgtctggggactgtacatttccggttcacgagtcgcgtttgaaacgaactcgattcagctgcaccaggtcctggcggtcaaaaccgcgcggaatggcgaagcgcaggtcccgttgggtcagtggtggacccgctga

SEQ ID NO: 12 - protein, 436 aa, Amycolicicoccus subflavus
MKAVLTAFTAPQLERMNVAEILSAVLGRDFPIRFTAYDGSALGPETARYGLHLTTPRGLTYLATAPGDLGLARAYVSGDLEVSGVHQGDPYEIMKILAHDVRVRRPSPATIASIMRSLGWERLRPVAPPPQENMPRWRRMALGLLHSKSRDAAAIHHHYDVSNEFYEHILGPSMTYTCAAYPSADSSLEEAQDNKYRLVFEKLGLKAGDRLLDVGCGWGGMVRFAAKRGVHVIGATLSRKQAEWAQKMIAHEGLGDLAEVRFCDYRDVTEAGFDAVSSIGLTEHIGLANYPSYFGFLKDKLRPGGRLLNHCITRPNNLQSNRAGDFIDRYVFPDGELAGPGFIISAVHDAGFEVRHEENLREHYALTLRDWNRNLARDWDACVHASDEGTARVWGLYISGSRVAFETNSIQLHQVLAVKTARNGEAQVPLGQWWTR

SEQ ID NO: 13 - DNA, 1548 bp, Corynebacterium glutamicum
  source 1..1548: mol_type = genomic DNA
atgagcggattagttgacccggatagtacttttttaaagaccatcggaaaactgagcaacagcttgtccattggtcgtggagtagatcaaaaagaggtaatccccaaaggctggaacgcccattgggaggcaattacaaagcttaagagaagctttgacgcgattcctgctggggagcgggtgcgtttagctaagaaaacctccaacctgttccgtggacgctccgatgcaggtcacggcctagatgtggcagcgcttgggggagtgattgccattgatccggtcaatgccaccgccgatgtacagggcatgtgcacgtatgaagacctggtagatgccactttaagttatggtctgatgccgttggttgtgcctcaactgaaaaccatcacgcttggtggcgcagtgaccggaatgggcgtggaatccacatccttccgcaacggtttgccacacgaatcagtgctggagatggatatttttaccggcactggtgagatcgtgacttgctcgcccacagaaaatgtcgacctttacagaggttttcccaactcttatggttcgctgggatacgcggtgcggctaaaaattgagctggaaccagtgcaagattacgtccagctgcgccacgtgcgcttcaacgatttagagtctttgaccaaagcgattgaggaagtcgcgtcttctctggagtttgataaccaacccgtcgattaccttgacggcgtggtgttttcacccacggaagcctacttagttcttggcacgcaaacctcacaacctggccccaccagcgattacaccagggatttaagctactaccgctccctgcaacacccagagggcatcacctatgaccgcctgacaatccgcgattacatctggcgctgggacaccgactggttctggtgttcacgcgcattcggcacccaaaaccccgtggtgcgcaaactctggcccagggatctgctgcgctcgagtttctattggaagatcatcggctgggatcgaaaatactccatcgctgatcgcctggaagagcgcaaaggccgcccggctagggaacgggtggtccaagacgtggaagttacgattgataaactgccagaatttttgaaatggttctttgaaagcagcgacatcgagccgctgtggctgtgcccgatcaagcttcgggaggtaccaggtagttcggttggtgctggagaaattttgagctccgctgaagcaatcgactccggtgctgctgaacacccttggccgctgtatcccttgaagaaggacgtgctgtgggtcaacatcggattctggtcctcagtgccggttgatctgatgggctccgatgcaccagagggagcatttaacagagaaatcgaacgcgtcatggcagagctaggcggacataaatcgctgtactccgaagcgttctacaccagggaagactttgaaaaactttatggcggaaccatcccggcgctgctaaaaaagcagtgggatccccacagccgattccccggtttgtatgaaaagacagtaaaaggcgcctag

SEQ ID NO: 14 - protein, 515 aa, Corynebacterium glutamicum
MSGLVDPDSTFLKTIGKLSNSLSIGRGVDQKEVIPKGWNAHWEAITKLKRSFDAIPAGERVRLAKKTSNLFRGRSDAGHGLDVAALGGVIAIDPVNATADVQGMCTYEDLVDATLSYGLMPLVVPQLKTITLGGAVTGMGVESTSFRNGLPHESVLEMDIFTGTGEIVTCSPTENVDLYRGFPNSYGSLGYAVRLKIELEPVQDYVQLRHVRFNDLESLTKAIEEVASSLEFDNQPVDYLDGVVFSPTEAYLVLGTQTSQPGPTSDYTRDLSYYRSLQHPEGITYDRLTIRDYIWRWDTDWFWCSRAFGTQNPVVRKLWPRDLLRSSFYWKIIGWDRKYSIADRLEERKGRPARERVVQDVEVTIDKLPEFLKWFFESSDIEPLWLCPIKLREVPGSSVGAGEILSSAEAIDSGAAEHPWPLYPLKKDVLWVNIGFWSSVPVDLMGSDAPEGAFNREIERVMAELGGHKSLYSEAFYTREDFEKLYGGTIPALLKKQWDPHSRFPGLYEKTVKGA

SEQ ID NO: 15 - DNA, 1308 bp, Corynebacterium glutamicum
  source 1..1308: mol_type = genomic DNA
atgagtaacgccgtagcgcaggacctcatgaccatcgccgacatcgtcgaggccacgaccactgcacccatcccattccacatcactgccttcgatggaagcttcactggccctgaagatgctccctaccagctgtttgttgccaacacggatgcagtatcctacatcgcaacagcgccaggagatttgggtttggcacgtgcctacctcatgggagacctcatcgtggaaggtgagcatcccggccatccttatgggatctttgatgcgttgaaggagttctaccgctgcttcaaacgcccagatgcatccaccaccttgcagatcatgtggactctgcggaaaatgaatgccttaaaattccaggaaattccaccaatggaacaagcccctgcatggcgtaaagcactgatcaacgggctagcatccaggcactcgaaatcccgcgacaagaaagccattagctaccactacgacgtgggcaatgagttctactccctgtttttagatgattccatgacctatacctgcgcgtattatccaacgccagaatcaagtttggaagaagcccaagaaaacaaataccgcctcatctttgaaaaactgcgtctgaaagaaggcgatcgcctcctagacgtgggatgcggttggggaggcatggtccgctacgccgccaaacacggtgtgaaagccatcggagttacgctgtctgaacagcaatatgagtggggtcaagcagagatcaaacgccaaggtttggaagacctcgcggaaattcgcttcatggattaccgcgatgttccagaaactggattcgatgcgatctcagcaatcggcatcattgaacacatcggtgtgaacaactatcccgactactttgaattgctcagcagcaaactcaaaacaggcggactgatgctcaaccacagcatcacctacccagacaaccgcccccgccacgcaggtgcatttattgatcgctacattttccccgacggtgaactcactggctctggcaccctgatcaagcacatgcaggacaacggtttcgaagtgctgcacgaagaaaacctccgctttgattaccaacgcaccctgcacgcgtggtgcgaaaacctcaaagaaaattgggaggaagcagttgaactcgccggtgaacccactgcacgactctttggcctgtacatggcaggttcggaatggggatttgcccacaacatcgtccagctgcaccaagtactgggtgtgaaactcgatgagcagggaagtcgcggagaagttcctgaaagaatgtggtggactatctaa

SEQ ID NO: 16 - protein, 435 aa, Corynebacterium glutamicum
MSNAVAQDLMTIADIVEATTTAPIPFHITAFDGSFTGPEDAPYQLFVANTDAVSYIATAPGDLGLARAYLMGDLIVEGEHPGHPYGIFDALKEFYRCFKRPDASTTLQIMWTLRKMNALKFQEIPPMEQAPAWRKALINGLASRHSKSRDKKAISYHYDVGNEFYSLFLDDSMTYTCAYYPTPESSLEEAQENKYRLIFEKLRLKEGDRLLDVGCGWGGMVRYAAKHGVKAIGVTLSEQQYEWGQAEIKRQGLEDLAEIRFMDYRDVPETGFDAISAIGIIEHIGVNNYPDYFELLSSKLKTGGLMLNHSITYPDNRPRHAGAFIDRYIFPDGELTGSGTLIKHMQDNGFEVLHEENLRFDYQRTLHAWCENLKENWEEAVELAGEPTARLFGLYMAGSEWGFAHNIVQLHQVLGVKLDEQGSRGEVPERMWWTI

SEQ ID NO: 17 - DNA, 1458 bp, Corynebacterium glyciniphilum
  source 1..1458: mol_type = genomic DNA
gtgaccgtcgccggcaggatcactgacgcggtacgcataggaaatggacttgaccagcgagatctagcccccgtcgggtggtacgcacacgaacaggccgtggcgcgactgaaggccagtttcgacgcggtccccgccgggcgtcgcgtgcggctggcgaagaagacgtccaaccttttccgcgggcgttccggcgaggcagtcgggctcgacgtgtcggggctgcacggcgtcatcgccgtcgaccccgttgagggcaccgctgacgtccagggcatgtgcacgtacgaggacctggtggacgtcctgctgccctacggtctggcgcccaccgtcgttccgcagctgaagaccatcactctcggcggtgcggtgaccggcatgggggtggaatccacctccttccgcaacggcctgccgcacgaagccgtcctggaaatggatgtgctcaccggtaccggagacatcctcacctgttcgccgacccagaacaccgacctctaccgcggcttccccaactcctacggttccctgggatacagcgtgcggctgaaggtgcggtgcgaacgggtggaaccctacgtcgacctgcggcatgtacgcttcgatgacgttcagtcgctcaccgacgccctcgacaacatcgtcgtggacaaggagtacgagggtgaacgggtcgactatctcgacggtgtggtcttcagcctggaggagagctacctcgtcctgggacgggcgaccagcgaggccggccccgttagcgactacacccgcgagcgcagttactaccgttctctgcagcatccgtcgggggtcctgcgcgacaagttgaccatccgcgactacctctggcggtgggacgtcgactggttctggtgcaaccgggccttcggtacccagaaccccaccatccgtactctgtggccgcgggatctcctgcggtcgagcttctactggaagatcatcggctgggaccgacgcttcgacatcgcggaccggatcgaggcacacaacgggcgccccgcacgcgagcgcgtcgtccaggacatcgaggtcacccccgacaacctgccggagttcctcacgtggttcttcacccactgcgagatcgagccggtgtggctgtgccccattcgactggccgacgactcgggcgagcggacaccgtggcccctgtacccgctgtcacccggcgacacctgggtcaacgtgggattctggagctcggtgcccgccgacctgatggggaaggacgccccgaccggagccttcaaccgggaggtggagagagtcgtctcggacctcggcggacacaagtcgttgtactccgaggcattctattctgaggaacagttcgccgccctctacggcggtgaacgtcccgcacaactcaaggcggtcttcgacccggatgaccggttccccgggttgtacgagaagaccgtgggcggcgtctga

SEQ ID NO: 18 - protein, 485 aa, Corynebacterium glyciniphilum
VTVAGRITDAVRIGNGLDQRDLAPVGWYAHEQAVARLKASFDAVPAGRRVRLAKKTSNLFRGRSGEAVGLDVSGLHGVIAVDPVEGTADVQGMCTYEDLVDVLLPYGLAPTVVPQLKTITLGGAVTGMGVESTSFRNGLPHEAVLEMDVLTGTGDILTCSPTQNTDLYRGFPNSYGSLGYSVRLKVRCERVEPYVDLRHVRFDDVQSLTDALDNIVVDKEYEGERVDYLDGVVFSLEESYLVLGRATSEAGPVSDYTRERSYYRSLQHPSGVLRDKLTIRDYLWRWDVDWFWCNRAFGTQNPTIRTLWPRDLLRSSFYWKIIGWDRRFDIADRIEAHNGRPARERVVQDIEVTPDNLPEFLTWFFTHCEIEPVWLCPIRLADDSGERTPWPLYPLSPGDTWVNVGFWSSVPADLMGKDAPTGAFNREVERVVSDLGGHKSLYSEAFYSEEQFAALYGGERPAQLKAVFDPDDRFPGLYEKTVGGV

SEQ ID NO: 19 - DNA, 1368 bp, Corynebacterium glyciniphilum
  source 1..1368: mol_type = genomic DNA
atgagcaggggattcacgccgctgacggtgggacagatcgtggacaaggtcatcacaccgccggcaccgttccgggtgaccgctttcgacggatccaccgcggggccggcagacgcggaactggcactggagatcacatcgccggacgccctggcctatatcgtgaccgcgccgggcgacctcggactggcacgcgcctacatcaccggaagcctccgcgtcaccggtgacgagcccggccacccgtacctcgtctttgaccacctccagcacctttacgaccagatccgacgcccctcggcgaaggacctgctggatatcgcccgctcgctgaaggccatgggggcgatcaaggtgcagccggcaccggagcaggagaccctcccgggctggaagagggccatactcgaggggctgtcccggcactctccggaacgggacaaggaggtcgtgagccgccactacgacgtgggcaatgacttctacgagctcttcctcggcgattccatggcctacacctgtgcctactatcccgagtttgacggtgagaaccaggtcaccggtcccaccggcgggtggcggtacgacgactgggagaaagggccgaccgccaacgggccgttgacccaggcgcaggacaacaagcatcgcctggtcttcgacaagctgcgactcaacccgggtgaccggttgttggacgtcggctgcgggtggggcggtatggtgcggtacgccgcccgccacggcgtgaaggccatcggtgtcacgctgtcccgagagcagtacgagtggggtaaggcgaagatcgaggaggagggtctgcaggacctcgccgaggtccggtgtatggactaccgtgacgtgccggagtccgacttcgacgcggtcagtgccatcggcatcctcgagcacatcggcgtgcccaactacgaggactacttcacccgcctgttcgccaagctgcgcccgggcggtcggatgctgaaccactgcatcacccgtccgcacaaccggaagacgaagaccggccagttcatcgaccgctacatcttccccgacggtgagctgaccggctcgggccggatcatcacgatcatgcaggacaccggattcgacgtcgtccacgaggagaatctgcgaccgcactaccagcgcacgttgcatgactggtgtgaactgttggccaccaactgggaccaggccgtccatctcgtgggcgaggagacggctcgtctgttcggcctgtacatggcggggtcggaatggggtttcgaacacaacgtgatccagctccaccaggttctcggcgtgaagccggacgcggcaggcagttccggggtgccggtccgccagtggtggaggtcctga

SEQ ID NO: 20 - protein, 455 aa, Corynebacterium glyciniphilum
MSRGFTPLTVGQIVDKVITPPAPFRVTAFDGSTAGPADAELALEITSPDALAYIVTAPGDLGLARAYITGSLRVTGDEPGHPYLVFDHLQHLYDQIRRPSAKDLLDIARSLKAMGAIKVQPAPEQETLPGWKRAILEGLSRHSPERDKEVVSRHYDVGNDFYELFLGDSMAYTCAYYPEFDGENQVTGPTGGWRYDDWEKGPTANGPLTQAQDNKHRLVFDKLRLNPGDRLLDVGCGWGGMVRYAARHGVKAIGVTLSREQYEWGKAKIEEEGLQDLAEVRCMDYRDVPESDFDAVSAIGILEHIGVPNYEDYFTRLFAKLRPGGRMLNHCITRPHNRKTKTGQFIDRYIFPDGELTGSGRIITIMQDTGFDVVHEENLRPHYQRTLHDWCELLATNWDQAVHLVGEETARLFGLYMAGSEWGFEHNVIQLHQVLGVKPDAAGSSGVPVRQWWRS

SEQ ID NO: 21 - DNA, 588 bp, Corynebacterium glyciniphilum
  source 1..588: mol_type = genomic DNA
gtggcggtgctgtgcacaccgttgctgctcggagcctgcaccatcggcgacgcgggaccgggggacgagaccacggaccctgtcgtggacactgaagcaccgcccgataaaccggtgccggactctgcggcggaatccggcgctgaagacggacctgattctgaggtgccggacgaccccgaccagcctgatgctgagccggtggagactgatcccgacgccccgggggcccggggactggcgatcggtgactgcgtcgccgacatggaccagctcgacggcaccggcgacatcgacgtcgtcgactgcgccggcccccatgccggcgaggtgtacgcacaggcggatatcgcaggtaagaacctgttccccggcaacgagccgttggggcaggaggcgggagcgatctgcgggggtgactccttcaccggctatgtcggcatcggattccccgagtcctcgctggacgtcgtcacgatgatgccgtccaaggagagctgggcgcaggaggaccggacggtgacctgtgtggtcaccgacccgaacctcgagcagatcgccggcacgctcgagcagagctggcgttag

SEQ ID NO: 22 - protein, 195 aa, Corynebacterium glyciniphilum
VAVLCTPLLLGACTIGDAGPGDETTDPVVDTEAPPDKPVPDSAAESGAEDGPDSEVPDDPDQPDAEPVETDPDAPGARGLAIGDCVADMDQLDGTGDIDVVDCAGPHAGEVYAQADIAGKNLFPGNEPLGQEAGAICGGDSFTGYVGIGFPESSLDVVTMMPSKESWAQEDRTVTCVVTDPNLEQIAGTLEQSWR

SEQ ID NO: 23 - DNA, 1395 bp, Knoellia aerolata
  source 1..1395: mol_type = genomic DNA
atgagcatggaccggaccggaccggccagggtgcggaccgtgggggagcggcggctgctcgagagcttcgccgccgtccccccgggcgaacgcgtgcggctggccaagcgcacgtccaacctcttccgcgcccgggagggcacctcgacacgcgggctcgacacgagcggactgaccggcgtgcgcgtggtcgacgcaggcaccctcacggccgacgtcgacggaatgtgcacgtacgaggacctcgtcgccgcaacgctgccgctcgggctcgcgccgctcgtcgtgccccagctgcggaccatcaccgtcggcggggcggtcaccggtctcgggatcgagtcgacgtcgttccgcaacgggttgccgcacgagtccgtcctcgagatggacgtcctcacgggtgccggcgagatcgtcactgccacagcggacaacgagcacgccgacctcttccgcggcttccccaactcctacgggtcgctgggctacgcgacgtgcctgcgcatcgagctcgagcgtgtgggtacctgtgtggaggtgaggcacgtccgcttccacgacctcgacgccctgtgcgccgccatcgccgaggtcgtggcgacgagatcgcacgagggcgaggaggtcgaccacgtggacggggtggtcttctcccgcgacgaggcgtacctcacgctgggtcgtcactccgaccggaccggaccgaccagcgactacaccgggcagcaggtctactaccggtcgatccagcacgacggcccctctccacggcgcgacctgctcaccactcacgactacctctggcgctgggacaccgactggttctggtgctcgcgcgccttcggggcccaggacccgcgcgtccggcggtggtggccgcgccggtggcgccggtcgagcgtgtactggaggctcgtggcggcggaccggcgcgtcgggttctcggaccgcctcgaggcacgtcggggcaacccgccgcgggagcgggtggtccaggacgtcgagatcccgctcgggcagaccgcggccttcctccactggttcctcgacgaggtgccgatcgaaccgatctggctgtgcccgttgcgtcttcgcgaccatcagaggtggccgctctatccgctcgagcccggacgcacctacgtcaacgtggggttctggtcgaccgtgccggggcccggaccgggcgaggagctgggcgccaccaaccgcgccatcgagcgccgtgtcgacgaggtcggcggccacaagtccctgtactccgactcctactactcccggtccgacttcgacgccctctacggcggggacgcgtatgccgtgctgaaggccacctacgacccggacgggcggttccctcacctctacgacaaggcggtgcgacacgcatga

SEQ ID NO: 24 - protein, 464 aa, Knoellia aerolata
MSMDRTGPARVRTVGERRLLESFAAVPPGERVRLAKRTSNLFRAREGTSTRGLDTSGLTGVRVVDAGTLTADVDGMCTYEDLVAATLPLGLAPLVVPQLRTITVGGAVTGLGIESTSFRNGLPHESVLEMDVLTGAGEIVTATADNEHADLFRGFPNSYGSLGYATCLRIELERVGTCVEVRHVRFHDLDALCAAIAEVVATRSHEGEEVDHVDGVVFSRDEAYLTLGRHSDRTGPTSDYTGQQVYYRSIQHDGPSPRRDLLTTHDYLWRWDTDWFWCSRAFGAQDPRVRRWWPRRWRRSSVYWRLVAADRRVGFSDRLEARRGNPPRERVVQDVEIPLGQTAAFLHWFLDEVPIEPIWLCPLRLRDHQRWPLYPLEPGRTYVNVGFWSTVPGPGPGEELGATNRAIERRVDEVGGHKSLYSDSYYSRSDFDALYGGDAYAVLKATYDPDGRFPHLYDKAVRHA

SEQ ID NO: 25 - DNA, 1284 bp, Knoellia aerolata
  source 1..1284: mol_type = genomic DNA
atgagccacacgaccgatgagatccgcacggtcgccgacctcgtcgacgaggtggtcgtcggcccgctgccggtgcgggtcacggcctacgacgggtcgaagacggggccggacagcgccccgcgaaccatccacatcgccaaccagcgagcggtcgcctacctcgccaccgcgcccggggacctcggcatggcccgcgcctacaccaccggtgacctcgtcgtcgagggcgtgcacccgggcaacccctacgaggccctggtcgacctcgaacgtgtgcacttccgccgcccggacccgcggctgctcctcgacctcgcgcgcatcgtcgggccacgcaacctcgcgcccccgcccccgccgccgcaggaggctgtgccgaggtggcggcgggtggccgagggcctgcgccactcgtacgggcgggacagcgaggcgatccgccaccactacgacgtctccaaccacttctacgagcaggtgctcggcccgagcatgacctacacctgcgcggtcttccccgaccacgacaccgggctcgacgaggcgcaggaggagaagtaccgcctcgtcttcgagaagctcgcgctgcgtcccggtgaccggttgctcgacatcggctgcgggtggggcgggatggtccggtacgccgcacggcggggggtgcgagcgctcggcgtgacactgtccggtgagcaggcggcgtgggcacaggtcgccatcgcccgcgaggggctgggggagctcgccgccgtccggcacgaggactaccgccacgtcgccgagaccgggttcgacgccatctcctcgatcggcatcaccgagcacatcggggtgcgcaactaccccacgtacttcgactggatgctccaccacgtcaagccgggagggctcgtgctcaaccactgcatcaccagacccgagaaccgggccaagagcgtcggccggttcatcgaccgctacatcttccccgacggcgagctcaccgggtccggccggatcatcacgaccatgcaggacaacggtttcgaggtcgtgcactccgagaacctgcgagagcactacgccctcaccctggcggcctggggcgagaacctcgtcgagcactgggcctcctgcgtggccgacgtgggggaggggacggcgaaggtctggggcctctacctcgcgggctcgcgtcgtggcttcgagcgcaacgtcgtccagctgcaccaggtgctggccgcgaggccggtgccgtcccgactcccgcaggtgccgctgcgccagtggtggacctcgtga

SEQ ID NO: 26 - protein, 427 aa, Knoellia aerolata
MSHTTDEIRTVADLVDEVVVGPLPVRVTAYDGSKTGPDSAPRTIHIANQRAVAYLATAPGDLGMARAYTTGDLVVEGVHPGNPYEALVDLERVHFRRPDPRLLLDLARIVGPRNLAPPPPPPQEAVPRWRRVAEGLRHSYGRDSEAIRHHYDVSNHFYEQVLGPSMTYTCAVFPDHDTGLDEAQEEKYRLVFEKLALRPGDRLLDIGCGWGGMVRYAARRGVRALGVTLSGEQAAWAQVAIAREGLGELAAVRHEDYRHVAETGFDAISSIGITEHIGVRNYPTYFDWMLHHVKPGGLVLNHCITRPENRAKSVGRFIDRYIFPDGELTGSGRIITTMQDNGFEVVHSENLREHYALTLAAWGENLVEHWASCVADVGEGTAKVWGLYLAGSRRGFERNVVQLHQVLAARPVPSRLPQVPLRQWWTS

SEQ ID NO: 27 - DNA, 1392 bp, Mycobacterium austroafricanum
  source 1..1392: mol_type = genomic DNA
gtgtctgttccttcgaccgacgcacgttctgctcacgccgacggcgtgcagcggcttctcgccagctatcgggcgattccccaagacgccacggtccggctggccaaacccacgtcgaacctcttccgtgcccgcgcgaaaaccaggaccaagggtctggacacgtctgggttgacgaacgtgatcgcggtcgacgcggaggcacgcaccgccgatgtggcagggatgtgcacctacgaagacctggtcgcggccacgctgccgcatggactttcgccgctggtggtgccgcagttgaagacgatcaccctcggcggggcggtcaccggactcgggatcgagtccgcctcgttccgcaacggcctgccacacgaatcggttctcgagatggacgtcctcaccggcaccggtgatgtcgtgcgcgcctcccccgacgagaaccctgacctgtttcgggcgtttccgaattcctatggcacgttgggctattcggttcggctcaagatcgagctggaaccggtgaagccgttcgtcgcgctgcgccacctccgtttccattcgctgtcggctctcatcgaggcgatggaccgcatcgtcgaaaccggcggcctcaacggcgaaccggtggactacctcgacggcgtcgtgttcagtgccgaggagagttacctgtgcgtggggcagcgctccgcgacaccgggcccggtcagcgactacacgggcaagcagatctactaccgctcgattcagcacgacggcccgaccgatggcgccgagaagcacgaccggctgaccatccacgactacctgtggcgctgggacaccgactggttctggtgctcaagggcattcggcgcgcagaacccgcggatccggcgctggtggccgcgccggtaccggcgcagcagtgtgtactggaagctgatcggctacgaccggcgtttcggtatcgccgatcgcatcgagaagcgcaacggccgacccccgcgcgagcgggtggtccaggacatcgaggtgcccatcgagcggaccgtcgagtttctgcagtggtttctcgacaccgtgcccatcgaaccgatctggttgtgcccgttgcggctccgcgacgaccgcgattggcccctgtatccgatccgaccccaccacacctacgtcaacgtgggtttctggtcgtcggtgccggtgggcccggaggagggctacaccaacaggatgatcgaacggaaagtcagcgacctcgacggtcacaaatcgctgtattccgatgcgtactactcgccggaagagtttgattcgctctatggcggggagacgtacaagacggtgaagaagacatacgacccagactctcgtttcctggacctgtacggcaaagcagtggggcggcaatga

SEQ ID NO: 28 - protein, 463 aa, Mycobacterium austroafricanum
VSVPSTDARSAHADGVQRLLASYRAIPQDATVRLAKPTSNLFRARAKTRTKGLDTSGLTNVIAVDAEARTADVAGMCTYEDLVAATLPHGLSPLVVPQLKTITLGGAVTGLGIESASFRNGLPHESVLEMDVLTGTGDVVRASPDENPDLFRAFPNSYGTLGYSVRLKIELEPVKPFVALRHLRFHSLSALIEAMDRIVETGGLNGEPVDYLDGVVFSAEESYLCVGQRSATPGPVSDYTGKQIYYRSIQHDGPTDGAEKHDRLTIHDYLWRWDTDWFWCSRAFGAQNPRIRRWWPRRYRRSSVYWKLIGYDRRFGIADRIEKRNGRPPRERVVQDIEVPIERTVEFLQWFLDTVPIEPIWLCPLRLRDDRDWPLYPIRPHHTYVNVGFWSSVPVGPEEGYTNRMIERKVSDLDGHKSLYSDAYYSPEEFDSLYGGETYKTVKKTYDPDSRFLDLYGKAVGRQ

SEQ ID NO: 29 - DNA, 1323 bp, Mycobacterium austroafricanum
  source 1..1323: mol_type = genomic DNA
ttgacgacatttcgggacggcgcggccgacaccggcctgcacggagaccgcaagctcaccctggcggaggtcttggaggtcttcgcctcgggccgactgcctctgaagttcacggcgtacgacggcagcagcgcgggcccggacgacgccacgctcgggctggacctgctgaccccccgcgggaccacgtacctcgcaacggctcccggcgatctcggcctggcccgggcctacgtctccggtgacctgcagttgcagggggtgcaccctggcgacccgtacgacctgctcaacgcactggtgcagaaactggacttcaagcgaccgtccgcccgggtgctggcgcaggtcgtccgatcgatcgggatcgagcacctgaaaccgatcgcgccaccgccgcaggaggcgctgccgcggtggcggcgcatcgcagaaggactgcggcacagcaagacccgtgacgccgacgcgatccaccaccattacgatgtctccaacaccttctacgagtgggtgctcgggccgtcgatgacctacacctgcgcctgctacccgcatcccgacgccaccctcgaggaggcgcaggagaacaaatatcggctggtgttcgagaaactgcgcctcaagccgggcgaccgccttctcgacgtgggttgcgggtggggcggaatggtgcgctacgcggcccgtcacggcgtcaaggcgatcggggtgacgctgtccagggagcaggcgcagtgggcacgcgccgccatcgaacgggacggcctgggtgacctcgccgaggtccgccacagcgactaccgcgatgtgcgcgagtcccagttcgacgccgtgtcttcgctggggctcaccgagcacatcggggtcgccaactatccgtcgtacttccggttcctcaagtcgaagttgcgcccgggcggcctactgctcaaccactgcatcacccggcacaacaatcgcaccggccccgccgccgggggattcatcgaccggtatgtgttcccggacggggagctgaccggatcgggccggatcatcaccgagatccaggacgtcggtttggaggtgatgcacgaagagaacctgcgccggcactatgcgctgacacttcgggactggtgccggaatctggtgcagcactgggacgaagcggtcgcagaggtcggcctgcccaccgccaaggtgtggggtctgtacatggctgcctcgcgggtcggcttcgagcagaacagcattcagctgcatcaggtactggcggtgaagctcgacgaacgtggcggggacggcggtttgccgttgcggccctggtggaccgcgtag

SEQ ID NO: 30 - protein, 440 aa, Mycobacterium austroafricanum
LTTFRDGAADTGLHGDRKLTLAEVLEVFASGRLPLKFTAYDGSSAGPDDATLGLDLLTPRGTTYLATAPGDLGLARAYVSGDLQLQGVHPGDPYDLLNALVQKLDFKRPSARVLAQVVRSIGIEHLKPIAPPPQEALPRWRRIAEGLRHSKTRDADAIHHHYDVSNTFYEWVLGPSMTYTCACYPHPDATLEEAQENKYRLVFEKLRLKPGDRLLDVGCGWGGMVRYAARHGVKAIGVTLSREQAQWARAAIERDGLGDLAEVRHSDYRDVRESQFDAVSSLGLTEHIGVANYPSYFRFLKSKLRPGGLLLNHCITRHNNRTGPAAGGFIDRYVFPDGELTGSGRIITEIQDVGLEVMHEENLRRHYALTLRDWCRNLVQHWDEAVAEVGLPTAKVWGLYMAASRVGFEQNSIQLHQVLAVKLDERGGDGGLPLRPWWTA

SEQ ID NO: 31 - DNA, 381 bp, Mycobacterium austroafricanum
  source 1..381: mol_type = genomic DNA
gtgatccgctttctgctgcgcgtcgcggtctttctcggatcgtcggcgatcgggctactggtggccggctggctggtgccgggggtgtcgctgtcggtgctgggcttcgtcaccgcggtggtgatcttcacggtggcacaagggattctgtcgccgttcttcctgaagatggccagccgctacgcgtcggccttcctcggcggcatcggcctggtgtccacgttcgtggcgctgctgctcgcgtcgctgctgtccaacgggctcagcatccgcggcgtcgggtcgtggatcgcggccacggtggtggtctggctggtcacagccctggcgaccgtcgtgctgcccgttctggtgctgcgggagaagaagaaagcagcctga

SEQ ID NO: 32 - protein, 126 aa, Mycobacterium austroafricanum
VIRFLLRVAVFLGSSAIGLLVAGWLVPGVSLSVLGFVTAVVIFTVAQGILSPFFLKMASRYASAFLGGIGLVSTFVALLLASLLSNGLSIRGVGSWIAATVVVWLVTALATVVLPVLVLREKKKAA

SEQ ID NO: 33 - DNA, 1392 bp, Mycobacterium gilvum
  source 1..1392: mol_type = genomic DNA
gtgtctgttgccgtaaccgacgcacgatccgcctacgcccacggcgtgcagcggctggtcgcgagttaccgcgccatccccgccggcgccaccgtccgcctggccaaacccacgtccaacctgttccgcgccagggcgaagagcaccgcggcgggcctcgacacctccggcctgacacatgtgatcgccgtggaccccgagacgcgcaccgccgaggtcgcggggatgtgcacctacgaggacctggtggcggcgacgctgccccacgggctttcaccgctggtggtcccgcaactcaagacgatcaccctcggcggcgccgtcaccgggctcggcatcgagtcggcgtcgttccgcaacggccttccgcacgaatcggtcctggagatggacatcctcaccgggaccggcgacatcgtgcgcgccgcgcccgacgagaatcccgaccttttccgcaccttcccgaattcttatggaacgctgggttactcggttcggctgaagatcgagctggagccggtgaagccgttcgtggcgttacgccatctccgcttccactcactgtcgacactcatcgcgacgatggaccgcatcgtcgacaccgggagtctcgacggtgagcaggtcgactatctcgacggagtggtgttcagcgccgaggagagctacctgtgcgtcggaacacgttccgcgacaccgggtcctgtcagcgactacaccggcgagcacatcttctaccggtcgatccagcacgattgcccgaccgaaggcggacagaagcacgaccggctgacggcgcacgactacttctggcgctgggacaccgactggttctggtgctcaagggcattcggcgcgcagaacccgaaggtccgtcggtggtggccccgacggctccggcgcagcagcttctactggaagctcgtcggctacgaccagcgtttcggcatcgccgaccggatcgagaaacaccacggccggccaccgcgcgaacgcgtcgtccaggacgtcgaggtccccatcgagcgcaccgtcgaattcctgcagtggttcctcgacacgatcccgatagagccgctctggttgtgcccgttgcgacttcgcgatgacaacagctggtcgctgtacccgctccggccccatcgcacgtatgtcaacgtgggattctggtcgtcggtgcccgtcgggccggaggagggtcacaccaacaagctgatcgaacgcaggatcagcgagctggagggacacaagtcgctgtactccgacgccttctattcggccgacgagttcgacgcgctgtacggcggcgagatctaccggaccgtgaagaagacctacgacccagattctcgtttcctcgacctctatgcgaaggcggtgcgacggcaatga

SEQ ID NO: 34 - protein, 463 aa, Mycobacterium gilvum
VSVAVTDARSAYAHGVQRLVASYRAIPAGATVRLAKPTSNLFRARAKSTAAGLDTSGLTHVIAVDPETRTAEVAGMCTYEDLVAATLPHGLSPLVVPQLKTITLGGAVTGLGIESASFRNGLPHESVLEMDILTGTGDIVRAAPDENPDLFRTFPNSYGTLGYSVRLKIELEPVKPFVALRHLRFHSLSTLIATMDRIVDTGSLDGEQVDYLDGVVFSAEESYLCVGTRSATPGPVSDYTGEHIFYRSIQHDCPTEGGQKHDRLTAHDYFWRWDTDWFWCSRAFGAQNPKVRRWWPRRLRRSSFYWKLVGYDQRFGIADRIEKHHGRPPRERVVQDVEVPIERTVEFLQWFLDTIPIEPLWLCPLRLRDDNSWSLYPLRPHRTYVNVGFWSSVPVGPEEGHTNKLIERRISELEGHKSLYSDAFYSADEFDALYGGEIYRTVKKTYDPDSRFLDLYAKAVRRQ

SEQ ID NO: 35 - DNA, 1323 bp, Mycobacterium gilvum
  source 1..1323: mol_type = genomic DNA
atgacgacttttcgggaacataccgacagttcggcgtccgacccggatcggaaactcactttggcagaggtgttggagatcttcgccgcgggtcgccgtccgctgaagttcaccgcctatgacggaagtagttgcgggcctgaggatgcgacactgggcctcgacctgctgaccccgcggggcacgacctacctggccacggcgccgggtgatctcggcctggcgcgggcctacatcgccggcgatctgcgcctcagtggtgtgcatcccggcgatccccatgacctgctcacggcgctgacggaacgcctggagtacaggcgtccgccggtgcgagtgctggccaatgttctgcgctccatcgggatcgagcacctcaagcccgtcgcgccgccaccccaggagcacctgccgcggtggcggcggatcgcagaggggttgcggcacagcaagacccgtgacgctgaggccatccagcaccactacgacgtctcgaacacgttctactcatgggtcctgggtccgtcgatgacctacacctgcgcctgctatccacacccggatgccacgctggaggaggcgcaggagaacaagtaccggctggtgttcgagaagcttcgactcaagcccggtgaccggctgctcgacgtcggttgcggctggggcggaatggtccgctacgccgcccggcacggggtcaaggtcctgggggtgacgctgtcgaaggagcaggcgcagtgggcggccgacgcagtcgagcgggacggcctgggtgagttggccgaggtccgccacggcgactaccgcgacgtgcgcgagtcgcacttcgacgcagtgtcctcgctcgggctcaccgagcacatcggcgtcgcgaactacccgtcgtacttccgcttcctgaagtcgaaactgcggccgggtggcctgctgctcaaccactgcatcacccgaaacaacaaccggagtcacgccaccgcaggcggattcatcgatcgctatgtctttcccgacggggagctgacggggtcggggcgaatcatcaccgaaatgcaggacgtcggactcgaggtcgtgcacgaggagaatctgcgtcaccactacgcgctgacgctgcgcgactggagccgcaacctggtcgcgcactgggacgacgcggtgaccgaggtcggtctgccgactgccaaggtgtggggcctctacatcgccgcgtcgcgagtcggcttcgagcagaacgccattcagctgcaccaggtgctgtcggtcaagctcgacgagcgtggctcggacggcggactgccgttacgaccctggtggaacgcctag

SEQ ID NO: 36 - protein, 440 aa, Mycobacterium gilvum
MTTFREHTDSSASDPDRKLTLAEVLEIFAAGRRPLKFTAYDGSSCGPEDATLGLDLLTPRGTTYLATAPGDLGLARAYIAGDLRLSGVHPGDPHDLLTALTERLEYRRPPVRVLANVLRSIGIEHLKPVAPPPQEHLPRWRRIAEGLRHSKTRDAEAIQHHYDVSNTFYSWVLGPSMTYTCACYPHPDATLEEAQENKYRLVFEKLRLKPGDRLLDVGCGWGGMVRYAARHGVKVLGVTLSKEQAQWAADAVERDGLGELAEVRHGDYRDVRESHFDAVSSLGLTEHIGVANYPSYFRFLKSKLRPGGLLLNHCITRNNNRSHATAGGFIDRYVFPDGELTGSGRIITEMQDVGLEVVHEENLRHHYALTLRDWSRNLVAHWDDAVTEVGLPTAKVWGLYIAASRVGFEQNAIQLHQVLSVKLDERGSDGGLPLRPWWNA

SEQ ID NO: 37 - DNA, 387 bp, Mycobacterium gilvum
  source 1..387: mol_type = genomic DNA
atgatccggttcctgctgcgcatcgcggtctttctgggctcatcagcgatcgggctcctcgtcgccggatggctggtgcccggggtgtcgctgtcggtgtggggcttcgtcacggcagtggtgatcttcaccgtggcgcaggcgatcctgtccccgttcttcctcaagatggccagccgctacgcctcggcgttcctcggcgggatcggtctggtgtcgacgtttgccgcgctgctgctcgtctcgctgctgtccaacggtctgagcatccgcggcatcggatcctggatcgccgcaaccgtggtggtctggttggtgaccgccctggcgacgctggtgctgccgatgttggtgctgcgcgagaagaaaaccgcgtcgcgcgtctga

SEQ ID NO: 38 - protein, 128 aa, Mycobacterium gilvum
MIRFLLRIAVFLGSSAIGLLVAGWLVPGVSLSVWGFVTAVVIFTVAQAILSPFFLKMASRYASAFLGGIGLVSTFAALLLVSLLSNGLSIRGIGSWIAATVVVWLVTALATLVLPMLVLREKKTASRV

SEQ ID NO: 39 - DNA, 1425 bp, Mycobacterium sp.
  source 1..1425: mol_type = genomic DNA; note = subspecies indicus pranii
atgcacgggctgttgtcgaagactagggtatatgtggtgcctgtccttggatctgcactctcggcccacaagtcgggcgttgaccggctgctggcaagctatcgatccattcccgcaacgtccgcggtccggctggccaaaccgacgtcaaacctgttccgcgcccgcaccaaacgtgacgcgcccggcttggacacctcggggctgaccggcgtcctgagcgtggatcccgaaacccgcaccgcggacgtcgccggcatgtgcacctacgcggacctggtggccgcaacgctgccctacggcctgtcgccgctggtcgtcccgcagctgaagaccatcaccctcggcggggcggtcagcggcctggggatcgagtcggcgtcgtttcgcaacgggctgccgcacgaatcggtgctggagatggatatcctcaccggcgctggcgatttgctcaccgcatcacgtacccagcacccggacctgttccgcgccttcccgaattcctatgggacactggggtattcgacccggcttcggatcgagctggaacccgtcgcaccgttcgtcgcgctgcgccacatccgcttccgctcgctgcccgcgctgatcgccgcggccgaacgcatcgtcgacaccggcgggcagggcggaaccccggtcgactacctcgacggggtggtcttcagcgccgacgaaagctacctgtgcgtgggccggcggaccaccacccccggcccggtcagcgactacaccggcaaggacatctactaccagtccatccggcacgacgccccgggcctggaggcgaccaaggatgaccggctgaccatgcacgactacttctggcgctgggacaccgattggttctggtgctcgcgcgcgttcggcgtgcaggacccgcgggtgcgacgcttctggccgcgccgttatcggcgcagcagcttctactggaagctgatttccctggaccggcgcttcgggatctccgaccgcatcgaggcgcgcaacgggcggcccccacgcgaacgggtggtgcaagacatcgagattccaatcgaacggacctgcgacttcctggagtggttcctggacaacgtgccaatcacgccgatctggttgtgcccgttgcgccttcgcgaccgcgacggctggccgttgtacccgatgcggccggatcacacgtacgtcaacgtcggcttctggtcgtcggtgccggggggcgcgaccgagggcgccgccaaccggatgatcgaagaaaaggtgagcgaactcgacgggcacaagtccctgtactccgattccttctactcccgcgaggacttcgacgagctgtacggcggcgagacctacaacaccgtcaagaaaacctacgaccccgattctcgtttactcgacctctacgcaaaggcggtgcaacggcgatga

SEQ ID NO: 40 - protein, 474 aa, Mycobacterium sp.
  source 1..474: note = subspecies indicus pranii
MHGLLSKTRVYVVPVLGSALSAHKSGVDRLLASYRSIPATSAVRLAKPTSNLFRARTKRDAPGLDTSGLTGVLSVDPETRTADVAGMCTYADLVAATLPYGLSPLVVPQLKTITLGGAVSGLGIESASFRNGLPHESVLEMDILTGAGDLLTASRTQHPDLFRAFPNSYGTLGYSTRLRIELEPVAPFVALRHIRFRSLPALIAAAERIVDTGGQGGTPVDYLDGVVFSADESYLCVGRRTTTPGPVSDYTGKDIYYQSIRHDAPGLEATKDDRLTMHDYFWRWDTDWFWCSRAFGVQDPRVRRFWPRRYRRSSFYWKLISLDRRFGISDRIEARNGRPPRERVVQDIEIPIERTCDFLEWFLDNVPITPIWLCPLRLRDRDGWPLYPMRPDHTYVNVGFWSSVPGGATEGAANRMIEEKVSELDGHKSLYSDSFYSREDFDELYGGETYNTVKKTYDPDSRLLDLYAKAVQRR

SEQ ID NO: 41 - DNA, 1263 bp, Mycobacterium sp.
  source 1..1263: mol_type = genomic DNA; note = subspecies indicus pranii
atggccgagatcctggaggtcttcgccgccaccggccgacatccgctgaagttcaccgcctacgacggcagcatcgccggcaacgaggacgccgaactgggcctggaccttcgcagcccccgcggcgccacctatctggcgaccgcccccggcgaactcggcctcgcccgcgcctacgtgtcgggcgacctgcaggcctacggcgtccatcccggcgacccgtaccaactgctcaagacgctcaccgatcgggtggaattcaagcggcccccggtgcgggtgctggccaacgtcgtgcggtcgctggggttcgagcggttgctgccggtcgcgccgcccccgcaggaggcgctgccccggtggcggcgcatcgccgacgggctgatgcacacgaggacccgcgacgccgaggccatccaccaccactacgacgtgtccaacaccttctacgaattggtgttggggccgtcgatgacctacacctgcgcggtgtatcccgatgccgacgcgacactcgaacaggcgcaggagaacaagtaccggctgatcttcgagaagctgcggctgaaggcgggcgaccggctgctcgacgtcggctgcggctggggcggcatggtgcgctacgcggcccggcgcggcgtccgggccaccggcgccaccctgtcggccgaacaggcgaagtgggcgcagaaggcgatcgccgaggaaggccttgcggacctggccgaggtgcgccacaccgactatcgggacgtgggcgaggcggcgttcgacgccgtgtcctcgatcgggctgaccgagcacatcggcgtcaagaattaccccgcctacttcggcttcttgaagtcgaagctgcgcaccggcggcctgctgctcaatcactgcatcacccgccacgacaacacgtcgacgtcgttcgcgggcggattcaccgatcgctatgtcttcccggacggggagctgaccggctcgggccgcatcacctgcgacgtccaggactgcggcttcgaggtgctgcacgcggagaacttccgccaccactacgcgatgacgctgcgcgactggtgccgcaatctggtcgagaactgggacgccgcggtcagcgaggtcggcctaccgaccgcgaaggtctggggcctgtacatggcggcgtcacgggttgcgttcgagcagaacaaccttcagctgcatcacgtgctggcggccaagaccgacgcgcggggcgacgacgacctgccgctgcggccgtggtggacggcctga

SEQ ID NO: 42 - protein, 420 aa, Mycobacterium sp.
  source 1..420: note = subspecies indicus pranii
MAEILEVFAATGRHPLKFTAYDGSIAGNEDAELGLDLRSPRGATYLATAPGELGLARAYVSGDLQAYGVHPGDPYQLLKTLTDRVEFKRPPVRVLANVVRSLGFERLLPVAPPPQEALPRWRRIADGLMHTRTRDAEAIHHHYDVSNTFYELVLGPSMTYTCAVYPDADATLEQAQENKYRLIFEKLRLKAGDRLLDVGCGWGGMVRYAARRGVRATGATLSAEQAKWAQKAIAEEGLADLAEVRHTDYRDVGEAAFDAVSSIGLTEHIGVKNYPAYFGFLKSKLRTGGLLLNHCITRHDNTSTSFAGGFTDRYVFPDGELTGSGRITCDVQDCGFEVLHAENFRHHYAMTLRDWCRNLVENWDAAVSEVGLPTAKVWGLYMAASRVAFEQNNLQLHHVLAAKTDARGDDDLPLRPWWTA

SEQ ID NO: 43 - DNA, 1380 bp, Mycobacterium phlei
  source 1..1380: mol_type = genomic DNA
gtgtctgaaccccgaaccgacgcacgtgttgttcaggccgcgggcgtgcacaagctgctggagagctaccgcgcgatcccgcccgaggccaccgtccggctggccaaacccacctcgaacctgttccgggcgcgcgccaagacctcggtcaagggtctcgatgtctcgggcctgacccatgtgatctccgtcgaccccgacgagcgcaccgctgaggtggccgggatgtgcacctacgaggacctggtcgccgcgacgctgccgtacgggctgtcaccgctggtggtgccgcagctcaagaccatcaccctcggcggcgccgtgacgggtctgggcatcgagtcggcgtcgttccgtaacggcctgccgcacgagtcggtgctggagatggacatcctcaccggatcgggcgagatcctcaccgcctcccgcgaccagcaccccgacctgttccgggcgttcccgaactcctatggcacgctgggctattcggtgcggctgaagatcgagttggagaccgtcaaaccgttcgtcgcggtccgtcacctgcggttccacgacatcgaggacctggtcgccgagatggaccgcattgtcgagaccggcggctacgacggcaccccggtcgactatctcgacggtgtggtgttctcggcccgcgagagctacctgacgctgggcttccagaccgccaccccgggcccggtcagcgactacaccggccagcagatctactaccgctcgatccagcacgaggacggcgtcaaggacgaccggctgacgatccacgactacttctggcgctgggacaccgactggttctggtgctcgcgggcgttcggcgtgcagaacccgacgatccgccggttctggccgcgccggctcaagcgcagcagcttctactggaagctggtcgcctacgaccgcaagttcaacatcgccgatcgcatcgagatgcacaacggccgcccgccccgcgagcgcgtcgtgcaggacatcgaggtgccgatcgagcgggtcgccgagtttttgggctggttcctcgacaacgtgccgatcgagccgatctggctgtgcccgttgcgtcttcgcgacgacgccggctggccgctgtacccgatccgggcgcagcacacctacgtcaacgtggggttctggtcctcggtgccggtggggcccaccgaggggcacacgaaccggctgatcgagcgcaaggtcagcgagctcgacgggcacaagtcgctgtactcggacgcgtactactcgcgcgacgagttcgaccagctctacggcggcgaaatctacaaaaccgttaaaaaggcctacgatccagattcacgactgctcgacctgtacgcgaaggcggtgcagcgccagtga

SEQ ID NO: 44 - protein, 459 aa, Mycobacterium phlei
VSEPRTDARVVQAAGVHKLLESYRAIPPEATVRLAKPTSNLFRARAKTSVKGLDVSGLTHVISVDPDERTAEVAGMCTYEDLVAATLPYGLSPLVVPQLKTITLGGAVTGLGIESASFRNGLPHESVLEMDILTGSGEILTASRDQHPDLFRAFPNSYGTLGYSVRLKIELETVKPFVAVRHLRFHDIEDLVAEMDRIVETGGYDGTPVDYLDGVVFSARESYLTLGFQTATPGPVSDYTGQQIYYRSIQHEDGVKDDRLTIHDYFWRWDTDWFWCSRAFGVQNPTIRRFWPRRLKRSSFYWKLVAYDRKFNIADRIEMHNGRPPRERVVQDIEVPIERVAEFLGWFLDNVPIEPIWLCPLRLRDDAGWPLYPIRAQHTYVNVGFWSSVPVGPTEGHTNRLIERKVSELDGHKSLYSDAYYSRDEFDQLYGGEIYKTVKKAYDPDSRLLDLYAKAVQRQ

SEQ ID NO: 45 - DNA, 1314 bp, Mycobacterium phlei
  source 1..1314: mol_type = genomic DNA
atgacggcgatcaaagagaacccggtcctgacttcggccaggaagctgtccctggccgagattctggaaatccttgccgggggcgaactcccggtgcgtttcacggcctacgacggcagctcggcgggcccggcggactccccgctcggcctggagctgctgaccccgcgcggcaccacctatctggccaccgccccgggcgatctcgggctggcacgcgcctacatcgccggtgacctgcagccgcacggcgtgcatccgggcgatccgtacgagctgctcaaggccctgtcggagaagatggagttcaagcggccgcccgcgaaggtgctggccaacatcgtgcgctccatcggtatcgagcacctcaagccgatcgcaccgccgccgcaggaggcgcagccgcgctggcgccggatcgcggaagggttgcggcacagcaagactcgcgacgccgaggcgatccaccaccactacgacgtgtccaacacgttctacgagtgggtgctcggcccgtcgatgacctacacctgcgcgtgctacccggacgtcgacgcaaccctggagcaggcgcaggagaacaagtaccgcctggtgttcgagaagctgcgcctgaagccgggcgaccggctgctcgacgtgggctgcggctggggcggcatggtgcgctacgccgcccagcacggggtcaaggccatcggcgtcacgctgtctcgggagcaggcgacgtgggcgcagaaggcgatcgccgagcaggggctcagcgatctggccgaggtccgccacggcgactaccgcgacattcgcgagtccgggttcgacgcggtgtcctcgatcgggctgaccgagcacatcggcgtggccaactacccgtcgtacttccggttcctgcagtccaagctgcgtgtcggcgggctgctgctcaaccactgcatcacccggccggacaacaagtcgcaggccagcgcgggcgggttcatcgaccgctacgtgttccccgacggggagctcaccgggtccggccgcatcatcgccgcggcccaggacgtcggcctcgaggtggtgcacgaggagaacctgcgccagcactacgcgatgacgctgcgcgactggtgccgcaacctcgtcgagcactgggacgaggcggtcgccgaggtcggcctggaacgcgccaagatctggggcctgtacatggccggctcccggctcggcttcgagacgaacatcgtgcagctgcaccaggtgctggcggtcaagctggaccgcaggggcggcgacggcgggctgccgttgcgcccgtggtggacgccctag

SEQ ID NO: 46 - protein, 437 aa, Mycobacterium phlei
MTAIKENPVLTSARKLSLAEILEILAGGELPVRFTAYDGSSAGPADSPLGLELLTPRGTTYLATAPGDLGLARAYIAGDLQPHGVHPGDPYELLKALSEKMEFKRPPAKVLANIVRSIGIEHLKPIAPPPQEAQPRWRRIAEGLRHSKTRDAEAIHHHYDVSNTFYEWVLGPSMTYTCACYPDVDATLEQAQENKYRLVFEKLRLKPGDRLLDVGCGWGGMVRYAAQHGVKAIGVTLSREQATWAQKAIAEQGLSDLAEVRHGDYRDIRESGFDAVSSIGLTEHIGVANYPSYFRFLQSKLRVGGLLLNHCITRPDNKSQASAGGFIDRYVFPDGELTGSGRIIAAAQDVGLEVVHEENLRQHYAMTLRDWCRNLVEHWDEAVAEVGLERAKIWGLYMAGSRLGFETNIVQLHQVLAVKLDRRGGDGGLPLRPWWTP

SEQ ID NO: 47 - DNA, 1413 bp, Mycobacterium tuberculosis
  source 1..1413: mol_type = genomic DNA
atgcaggggcagttgtcgaggactagggtatatacggtgcctgtccctggatctgcacagtcggcttacgcctgcggcgtcgagcggttgctggcgagctatcgatccatccccgcgactgcatccatccggcttgccaagcccacctcaaatctgttccgcgcccgcgtcaaacacgatgcacgcggcctggacgcatcgggactgaccggtgtcatcggtatcgatcccgaggcccgcaccgccgacgtggccggcatgtgcacatacgaggacctaatcgccgcgacactgcactacggtctgtcaccattggtggttccgcagctgaggacgatcacattgggcggagcggtcaccggcttgggtatcgagtcggcgtcgttccgcaacggcctgccccacgagtcggtgctggagatggatatcctcaccggcgcaggagaacttctcaccgtctcgcccggacagcactccgacttgtaccgtgcattccctaactcgtatgggacactgggctattcaacccggcttcgaatccagctggagccggtccggccgtttgtcgcgctgcggcacatccgatttagctcgttgacggcgatggtggccgcaatggagcgcatcatcgacaccggcggactggacggcgaatcggtggactatctcgacggggtggttttcagcgctgacgaaagctacctgtgcatcggcatgcagacgagcgtaccgggcccggtcagcgactacaccggacaagacatctactaccggtcgatccaacacgaggcggggatcaaggaagaccggttgaccatccacgattacttctggcgctgggacaccgattggttctggtgctcacgatcgtttggtgcccaaaacccgcggctgcgccgctggtggccgcggcgctaccggcgtagcagtgtctactggaggttgatggcgctcgatcagcgcttcgggatcgccgaccggttcgagaacagcaggggtcgtcccgcgcgtgaacgggtggtgcaggatatcgaagtgccgatcgaacggacctgcgagtttctggagtggttcggggaaaacgtgcccatttcgccaatctggttgtgcccgttgcggctacgcgatcacgccggctggccgctgtacccgatccggcctgaccgtagctatgtcaacatcgggttctggtcgtcggtgccggttggcgccaccgagggcgccaccaaccgcaagatcgagaacaaggtgagtgcgctcgacgggcacaagtcgctctactccgactccttctatacccgcgaggagttcgacgagctctacggcggcgagacttacaacactgtgaagaaagcctacgatcccgattcgcgtctcctcgatctttacgcaaaggcggtgcaacgacgatga

SEQ ID NO: 48 - protein, 470 aa, Mycobacterium tuberculosis
MQGQLSRTRVYTVPVPGSAQSAYACGVERLLASYRSIPATASIRLAKPTSNLFRARVKHDARGLDASGLTGVIGIDPEARTADVAGMCTYEDLIAATLHYGLSPLVVPQLRTITLGGAVTGLGIESASFRNGLPHESVLEMDILTGAGELLTVSPGQHSDLYRAFPNSYGTLGYSTRLRIQLEPVRPFVALRHIRFSSLTAMVAAMERIIDTGGLDGESVDYLDGVVFSADESYLCIGMQTSVPGPVSDYTGQDIYYRSIQHEAGIKEDRLTIHDYFWRWDTDWFWCSRSFGAQNPRLRRWWPRRYRRSSVYWRLMALDQRFGIADRFENSRGRPARERVVQDIEVPIERTCEFLEWFGENVPISPIWLCPLRLRDHAGWPLYPIRPDRSYVNIGFWSSVPVGATEGATNRKIENKVSALDGHKSLYSDSFYTREEFDELYGGETYNTVKKAYDPDSRLLDLYAKAVQRR

SEQ ID NO: 49 - DNA, 1263 bp, Mycobacterium tuberculosis
  source 1..1263: mol_type = genomic DNA
atggccgagatcctggagatcttcaccgcgaccgggcaacacccgctgaagttcaccgcgtatgacggcagcaccgcgggacaagacgacgccacactgggcctggatcttcggacgccccgcggcgccacctacttagctaccgctcccggcgaactcggcctggcccgcgcttatgtgtcgggtgacctacaggcacacggagtacatcccggcgatccgtacgaactgctcaaaacgctgaccgaaagggtcgacttcaaacggccgtcggcgcgggtgctggctaatgtggtgcgctcgatcggcgttgagcacatactgcccatcgcgccgccaccccaggaggcgcgaccccggtggcgtcgaatggctaatggcttgctgcacagcaagacccgtgacgccgaggctatccatcaccactacgacgtctccaacaacttctacgagtgggtgctcgggccatcgatgacctacacgtgcgcggtgtttccgaacgctgaggcttcgctggagcaggcccaagagaacaaataccgactcattttcgaaaagctacggctagagccgggtgaccggctactcgacgtcggctgcggctggggcggcatggtgcgctacgccgcccgacgcggtgtccgggtgatcggcgccacgctctcggccgagcaggccaagtggggccagaaagcagtcgaggacgagggattgagcgacctcgcgcaggtgcggcattccgactaccgcgacgtagccgagaccggtttcgacgccgtttcttcgatcgggctaaccgagcacatcggcgtcaagaattacccgttctacttcgggtttctcaagtcgaagttgcgcaccggcggcttgctgctcaatcactgcatcacccgccacgacaacaggtcgacgtcctttgccggcgggttcaccgaccgttacgttttccccgacggggagctgacgggctcgggacgtattaccaccgagatccagcaggtcggcttggaagtgctgcacgaggagaacttccgccatcactacgcgatgacgctgcgcgactggtgcggcaacctcgtcgaacactgggacgacgcggtcgccgaggtcggtctgccgaccgccaaggtgtggggcctgtacatggcggcttcgcgggtggccttcgaacgaaacaacctgcagctacatcacgtattggcgaccaaggtggacccccggggcgacgacagcttgccactgcggccctggtggcagccctag

SEQ ID NO: 50 - protein, 420 aa, Mycobacterium tuberculosis
MAEILEIFTATGQHPLKFTAYDGSTAGQDDATLGLDLRTPRGATYLATAPGELGLARAYVSGDLQAHGVHPGDPYELLKTLTERVDFKRPSARVLANVVRSIGVEHILPIAPPPQEARPRWRRMANGLLHSKTRDAEAIHHHYDVSNNFYEWVLGPSMTYTCAVFPNAEASLEQAQENKYRLIFEKLRLEPGDRLLDVGCGWGGMVRYAARRGVRVIGATLSAEQAKWGQKAVEDEGLSDLAQVRHSDYRDVAETGFDAVSSIGLTEHIGVKNYPFYFGFLKSKLRTGGLLLNHCITRHDNRSTSFAGGFTDRYVFPDGELTGSGRITTEIQQVGLEVLHEENFRHHYAMTLRDWCGNLVEHWDDAVAEVGLPTAKVWGLYMAASRVAFERNNLQLHHVLATKVDPRGDDSLPLRPWWQP

SEQ ID NO: 51 - DNA, 1392 bp, Mycobacterium vanbaalenii; identical to SEQ ID NO: 27
  source 1..1392: mol_type = genomic DNA

SEQ ID NO: 52 - protein, 463 aa, Mycobacterium vanbaalenii; identical to SEQ ID NO: 28

SEQ ID NO: 53 - DNA, 1323 bp, Mycobacterium vanbaalenii; identical to SEQ ID NO: 29
  source 1..1323: mol_type = genomic DNA

SEQ ID NO: 54 - protein, 440 aa, Mycobacterium vanbaalenii
MTTFRDGAADTGLHGDRKLTLAEVLEVFASGRLPLKFTAYDGSSAGPDDATLGLDLLTPRGTTYLATAPGDLGLARAYVSGDLQLQGVHPGDPYDLLNALVQKLDFKRPSARVLAQVVRSIGIEHLKPIAPPPQEALPRWRRIAEGLRHSKTRDADAIHHHYDVSNTFYEWVLGPSMTYTCACYPHPDATLEEAQENKYRLVFEKLRLKPGDRLLDVGCGWGGMVRYAARHGVKAIGVTLSREQAQWARAAIERDGLGDLAEVRHSDYRDVRESQFDAVSSLGLTEHIGVANYPSYFRFLKSKLRPGGLLLNHCITRHNNRTGPAAGGFIDRYVFPDGELTGSGRIITEIQDVGLEVMHEENLRRHYALTLRDWCRNLVQHWDEAVAEVGLPTAKVWGLYMAASRVGFEQNSIQLHQVLAVKLDERGGDGGLPLRPWWTA

SEQ ID NO: 55 - DNA, 381 bp, Mycobacterium vanbaalenii; identical to SEQ ID NO: 31
  source 1..381: mol_type = genomic DNA

SEQ ID NO: 56 - protein, 126 aa, Mycobacterium vanbaalenii; identical to SEQ ID NO: 32

SEQ ID NO: 57 - DNA, 1452 bp, Rhodococcus opacus
  source 1..1452: mol_type = genomic DNA
atgcgggagggtggacgccccttccgtgcgcatcgcactctgcccgtcaccgggatcgacgctcaccgcgccggcgtcgaacggcttctcgcgtcctaccgcgcgattcccacggacgccaccgtgcgactcgcgaagaagacgtccaacctgttccgggcgcgggcccagaccagcgcacccggcctcgacgtctccgggctcggcggagtcatctcggtcgacgagcaggaccggaccgcggatgtcgccggaatgtgcacgtacgaagacctggtggacgccaccctcccgtacgggctggcgccgctggtggttccgcaactcaagaccatcacactcggcggcgcggtcaccggcctcggcatcgagtcgacgtcgttccgcaacgggctcccccacgaatcggtcctcgagatcgacgtcctgaccggaagcggcgacatcgtcaccgcgagaccggaaggcgagaactccgacctgttctgggggttccccaactcctacggaaccctcggctactccacccgactgcgcatccagctcgaacccgtcaaacggtatgtggcactgcgccatctgcgtttcgactccctggacgagctgcagtcggcaatggatcgcatcgtcaccgagcgcgtccacgacggcatccccgtcgactatctggacggcgtcgtgttcaccgcgtccgagagttacctgacactgggccatcagaccgacgagggcggccccgtcagcgactacaccgggcagaacatcttctaccggtccatccagcacagttccgtgaaccaccccaaaacggacaaactcaccatccgagactacctgtggcgctgggacaccgactggttctggtgctcgcgcgccttcggcgcccagaaccccaccatccgccggctgtggccgaagaacctcctccgcagcagcttctactggaagctcatcgccctcgaccacaagtacgacatcggcgaccgactcgagaagcgcaagggcaacccgccacgcgaacgcgtcgtgcaggacgtcgaagtgcccatcgagcgcaccgcggacttcgtccgctggttcctcgacgaaatcccgatcgaaccgctgtggctgtgcccgttgcggttgcgggaacctgcccccgccggcgcgtcctcgcaacgcccctggcccctgtaccccctcgaaccgaaacgcacgtacgtgaacatcggattctggtcatcggtgcccatcgttccgggccgacccgagggggccgcgaatcggctgatcgaagacaaggtcagtgacttcgacggacacaagtccctctactccgattcgtactattcacgcgaagatttcgaacgcctctactacggcggcgatcgatacacggaactgaaaaaacgctacgacccgaaatcacgattactggaccttttctccaaggcggtgcaacgtcgatga

SEQ ID NO: 58 - protein, 483 aa, Rhodococcus opacus
MREGGRPFRAHRTLPVTGIDAHRAGVERLLASYRAIPTDATVRLAKKTSNLFRARAQTSAPGLDVSGLGGVISVDEQDRTADVAGMCTYEDLVDATLPYGLAPLVVPQLKTITLGGAVTGLGIESTSFRNGLPHESVLEIDVLTGSGDIVTARPEGENSDLFWGFPNSYGTLGYSTRLRIQLEPVKRYVALRHLRFDSLDELQSAMDRIVTERVHDGIPVDYLDGVVFTASESYLTLGHQTDEGGPVSDYTGQNIFYRSIQHSSVNHPKTDKLTIRDYLWRWDTDWFWCSRAFGAQNPTIRRLWPKNLLRSSFYWKLIALDHKYDIGDRLEKRKGNPPRERVVQDVEVPIERTADFVRWFLDEIPIEPLWLCPLRLREPAPAGASSQRPWPLYPLEPKRTYVNIGFWSSVPIVPGRPEGAANRLIEDKVSDFDGHKSLYSDSYYSREDFERLYYGGDRYTELKKRYDPKSRLLDLFSKAVQRR

SEQ ID NO: 59 - DNA, 1302 bp, Rhodococcus opacus
  source 1..1302: mol_type = genomic DNA
atgacaactctgaaagcttcacgctcccaggaccacaagctgaccatcgcagagattctcgaaactctgtccgacggcatgctccccctgcggttctccgcctacgacggcagcgccgccggcccggaggacgccccctacggtctccacctcaagacgacccgaggcaccacctacctggcgaccgcccccggcgacctcggcatggcccgggcctacgtgtccggcgacctcgaggcccgcggcgtccaccccggcgacccgtacgagatcctccgcgtgatgggcgacgaactgcacttccgccgtccgtccgcgctcacgctcgccgccatcacgcgctcgctcggctgggatctgctgcgccccatcgcccctcccccgcaggagcatctcccgcggtggcgtcgagtcgcggaagggttgcggcactccaagtcccgcgacgccgaggtcatccaccaccactacgacgtctcgaacaccttctacgagtatgtcctcggcccgtccatgacgtacacgtgcgcctgctacgagaacgccgagcagaccctcgaagaggcacaggacaacaagtaccgcctcgtcttcgagaagctcggcctccagcccggcgaccgactgctcgacatcggttgcggctggggatcgatggtccggtacgccgcccgccgcggcgtcaaggtcatcggcgccaccctgtcccgagagcaggccgaatgggcacagaaggccatcgccgaagaaggactgtccgacctcgccgaggtccggttctccgactaccgtgacgtccccgagaccggattcgacgccatctcctcgatcggcctgaccgagcacatcggcgtcggcaactaccccgcctacttcggactgctgcagagcaagctccgcgagggcggccggctgctgaaccactgcatcacccggcccgacaaccagagtcaggcacgcgcgggcggcttcatcgaccggtacgtcttccccgacggcgaactcaccggctccggacgcatcatcaccgagatccagaacgtcggactcgaggtgcggcacgaggagaatctgcgcgagcactacgcactcaccctcgccggctggtgccagaacctcgtcgacaactgggacgcctgcgtcgccgaggtcggcgaaggcaccgcacgtgtgtggggtctctacatggccgggtcgcgactgggcttcgaacgcaacgtcgttcagctgcaccaggtcctcgccgtcaagctcggacccaagggcgaggcgcatgtgccgctgcgtccgtggtggaagtag

SEQ ID NO: 60 - protein, 433 aa, Rhodococcus opacus
MTTLKASRSQDHKLTIAEILETLSDGMLPLRFSAYDGSAAGPEDAPYGLHLKTTRGTTYLATAPGDLGMARAYVSGDLEARGVHPGDPYEILRVMGDELHFRRPSALTLAAITRSLGWDLLRPIAPPPQEHLPRWRRVAEGLRHSKSRDAEVIHHHYDVSNTFYEYVLGPSMTYTCACYENAEQTLEEAQDNKYRLVFEKLGLQPGDRLLDIGCGWGSMVRYAARRGVKVIGATLSREQAEWAQKAIAEEGLSDLAEVRFSDYRDVPETGFDAISSIGLTEHIGVGNYPAYFGLLQSKLREGGRLLNHCITRPDNQSQARAGGFIDRYVFPDGELTGSGRIITEIQNVGLEVRHEENLREHYALTLAGWCQNLVDNWDACVAEVGEGTARVWGLYMAGSRLGFERNVVQLHQVLAVKLGPKGEAHVPLRPWWK

SEQ ID NO: 61 - DNA, 1428 bp, Streptomyces regensis
  source 1..1428: mol_type = genomic DNA
atgatcacactggcaggccgggccggtgcgcgcgatcatgggtgtatggccttcggtgccgccatccccacggggtcgggacacgccgggtacgccgagcgcgtcgcaacccttcgcgcccacctggccgacctcccggaggggacgccggtccggctggcgaagggcacctcgaacctgttccggccgcggtcccgcgccacggcggggctcgacgtgtcggccttcgaccacgtgctgtcgatcgatccgcagaaccggaccgccgacgtcgagggcatggtcacctacgagcggctcgtcgacgcgacgttgccgcacggcctgatgccgctcgtcgttccgcagctcaagacgatcacgctgggcggggcggtcacgggactgggcatcgagtcgtcgtcgttccgcgagggcatgccccacgaatccgtggtggagatggacatcctcacgggtgcgggagacgtggtgaccgcgaccccggacggcgagcacagcgacctgttcttcgggttccccaactcctacggaacgctgggatacgcgctgcgcctgcggatcgaactcgcgccggtgcgcccgtacgtacgactcgaacacctgcgtttctccgatccggcacgctacttcgagcgcctggcgcgtgcgtgccgcgaccgggaggccgacttcgtcgacggcaccgtcttcgctcccgacgagctgtacctgacgttggccacgttcagcggcgagcccgacgaggtcagcgactacacgtggatggacgtctactaccgctcgatcagggagaagacggtcgaccatctgccgatccgcgactacctgtggcggtgggacaccgactggttctggtgttcgcgcgcgctcggagcgcagaaccggctcgtgcggctgctcgcgggtccacgtctgctgcgttccgatgtgtactggaagatcgtcggtttcgaacgcaggcaccggctgtgggagcgtgcgagccggctgctgggcaggcccgagcgcgaagcggtgatgcaggacatcgaggtgccggtgcaccgcgccgaggagttcctgacgttcctgcaccgggagatccccatcagtccggtgtggatctgcccgctgagtgggcgggacgcgcgccggtggccgctgtacgagctcgacccggacgagctgtacgtcaacttcggtttctggggcacggtgccgctcgagccaggcgaaccgcagggttcgcacaaccggcgggtggagaacgtggttaccgaactcgacggacggaaatccctgtactcggagagtttctacgaccgcgacacgttctggcggttgtacggagggaatcaaggacagacgtaccaggccctgaagcatcgctacgacccgaacgggagattgctggacctgtacgccaagtgcgttcaagcgaggtga

SEQ ID NO: 62 - protein, 475 aa, Streptomyces regensis
MITLAGRAGARDHGCMAFGAAIPTGSGHAGYAERVATLRAHLADLPEGTPVRLAKGTSNLFRPRSRATAGLDVSAFDHVLSIDPQNRTADVEGMVTYERLVDATLPHGLMPLVVPQLKTITLGGAVTGLGIESSSFREGMPHESVVEMDILTGAGDVVTATPDGEHSDLFFGFPNSYGTLGYALRLRIELAPVRPYVRLEHLRFSDPARYFERLARACRDREADFVDGTVFAPDELYLTLATFSGEPDEVSDYTWMDVYYRSIREKTVDHLPIRDYLWRWDTDWFWCSRALGAQNRLVRLLAGPRLLRSDVYWKIVGFERRHRLWERASRLLGRPEREAVMQDIEVPVHRAEEFLTFLHREIPISPVWICPLSGRDARRWPLYELDPDELYVNFGFWGTVPLEPGEPQGSHNRRVENVVTELDGRKSLYSESFYDRDTFWRLYGGNQGQTYQALKHRYDPNGRLLDLYAKCVQAR

SEQ ID NO: 63 - DNA, 1317 bp, Streptomyces regensis
  source 1..1317: mol_type = genomic DNA
ttggcgtcgtcggggccaccgctgcccgccagggcggggtcccgatcggctgactcgacggcgttggacgcgatcctgcgccgcgtgctcggggacgacccgcccgtggccgtgaccgcgttcgacggcacggtggtcggtgacccggactcggcgctgcagctgcacatccgcacgccgacggccctgagctacgtgctcaccgcgcccaacgaactcgggttggcgcgggcctacgtcacgggacatctcgacgtgaccggcgacgtctaccaggtgctgcgcgcactgacgagcgtggccgagaacctcacgacggccgatcggatgtggctggccggccgtctcgcacgggacttcaccgaccggctgcggccggtgccgatccccgtcgaggaggcgccgtcgcggctccgcaggaccgcacgtggcctccggcattccaaggcgcgcgacagcgacgcgatctcccggcactacgacgtctcgaaccgcttctacgagctggtgctcggcccgtcgatggcctacacgtgcgcctgctacccggaggatgcggccacgctggagcaggcacagttccacaagttcgacctcgtgtgccgaaagctcggtctgaagccggggatgcgcctgctcgacgtgggctgcggttggggcggcatggtcgcccacgccgtggagcactacggggtgcgggcgatcggcgtcaccctctcgcgccagcaggcggagtggggacagcgggacctcgaggccaggggcctggccgatcgcggcgagatccgccatctggactaccgcgacgtgcccgagaccgggttcgacgcggtgtcgtccatcgggctcaccgaacacatcggcgcgcggaacctgccgtcgtacttccgcttcctgcactcgaagttgcgtcccggcggacggttgctcaaccactgcatcgtgcgcccgcacacctacgactcccatcggacgggcccgttcatcgaccgctacgtcttcccggacggcgaactcgagggcgtcgggacgatcgtgtcggcgatgcaggaccacgggttcgaggtacggcacgcggagaacctgcgggaacactacgggcgcaccctcgcggcgtggtgcgccaatctcgacgcgcactgggaggcggcggtggccgaggcgggcgtgcagcgggccagggtgtgggcgctgtacatggcggcctcccggctgtcgttcgaacgtcatgagctcgagctgcagcaggtgctcggcgtgaaacccgacgccgcgggcgggtcgtcgatgccgcttcgcccggactggggggtgtga

SEQ ID NO: 64 - protein, 438 aa, Streptomyces regensis
LASSGPPLPARAGSRSADSTALDAILRRVLGDDPPVAVTAFDGTVVGDPDSALQLHIRTPTALSYVLTAPNELGLARAYVTGHLDVTGDVYQVLRALTSVAENLTTADRMWLAGRLARDFTDRLRPVPIPVEEAPSRLRRTARGLRHSKARDSDAISRHYDVSNRFYELVLGPSMAYTCACYPEDAATLEQAQFHKFDLVCRKLGLKPGMRLLDVGCGWGGMVAHAVEHYGVRAIGVTLSRQQAEWGQRDLEARGLADRGEIRHLDYRDVPETGFDAVSSIGLTEHIGARNLPSYFRFLHSKLRPGGRLLNHCIVRPHTYDSHRTGPFIDRYVFPDGELEGVGTIVSAMQDHGFEVRHAENLREHYGRTLAAWCANLDAHWEAAVAEAGVQRARVWALYMAASRLSFERHELELQQVLGVKPDAAGGSSMPLRPDWGV

SEQ ID NO: 65 - DNA, 501 bp, Streptomyces regensis
  source 1..501: mol_type = genomic DNA
gtgcgcgtggcaccgccccgcatcggtgccacacccggcgcggtgggcgcaccggactacgcctccgccttccgcgtgccgacggcggcggcccgcaggcgttcgccgcgggaatggacgcgtgcggtgttcgagggcgcgcccgcgccgttggcgctgttcgtgcgttggggatggctggccgtgctccggttgcgcctcagtgaggaccccgaggcggtggcgggctggagacccacgacgctcgaccccggcacctccgacgcccccgacacctctgagacagccggaaactccgacgctgccgcactggaggccgaatcgccgctgctggaggcgtgcaacgtggcgttcgtcgacgacgacggtgtcacgtgggcgacctacgtccggttccgtggtggcctcggccgcgcggtgtgggcggtggcggcgcggatccaccacgtcgtcatcccctacctgctgcggcgggcggtgcggcgcacggaacgggagtga

SEQ ID NO: 66 - protein, 166 aa, Streptomyces regensis
VRVAPPRIGATPGAVGAPDYASAFRVPTAAARRRSPREWTRAVFEGAPAPLALFVRWGWLAVLRLRLSEDPEAVAGWRPTTLDPGTSDAPDTSETAGNSDAAALEAESPLLEACNVAFVDDDGVTWATYVRFRGGLGRAVWAVAARIHHVVIPYLLRRAVRRTERE

SEQ ID NO: 67 - DNA, 1413 bp, Thermobifida fusca
  source 1..1413: mol_type = genomic DNA
gtgaactgtcagtcttccgcgtccaacctcgccaaccacatcaacgcggtgtacgagctgcgccgcgcctatgcgcggctgtccgccgacaagccggtgcgcctggcgaagaccacctccaacctcttccgcttccgcagccgggacgatgccgcgcgtctcgacgtcagcgctttcacctcggtgatcagcatcgacacggaggcgcgggtcgcggaggtgggcggcatgaccacctacgaggacctggtcgccgccaccctgcggcatggcctgatgccgccggtggttccgcaactgcgcacgatcaccctgggcggtgcggtcaccgggctggggatcgaatcctcgtccttccgcaacgggctcccgcacgagtcagtggaagagatggagatcctcaccggcagcggccaggtggtggtggcccggcgcgacaacgagcaccgcgacctgttctacggtttccccaactcgtacggcaccctcggttacgcgctgcggctccgcatccagctcgaaccggtccgcccctacgtccacctgcggcacctgcggttcaccgatgccgcagcggccatggccgcgctggagcagatctgcgcggaccgcacccacgacggggagaccgtcgacttcgtcgacggcgtcgtgttcgcccgcaacgagctgtacctgaccttggggacgttcaccgaccgggctccgtggaccagcgactacaccggaaccgacatctactaccggtcgatcccccgctacgcgggccccggccccggcgactacctcaccacgcacgactacctgtggcggtgggacaccgactggttctggtgctcccgcgccttcggactgcagcatcccgtggtgcgccgcctgtggccgcgttccttgaaacgctccgacgtctaccgcaagctcgtcgcctgggaccggcgcactgacgcgagccgcctgctcgactactaccgcgggcgcccgcccaaggaaccggtgatccaggacatcgaggttgaggtggggcgggctgccgagttcctcgacttcttccacaccgagatcggcatgtccccggtgtggctgtgcccgctgcggctgcgagaagacacagccgacgatacggaaccggtctggccgctctaccccctcaaaccccgccgcctctacgtcaacttcgggttttggggcctcgttccgatccgtcccggtggaggcaggacataccacaaccggctgatcgaaaaagaagtgacccggttgggcgggcacaagtcgctctactcggacgccttctacgacgaggacgagttctgggagctctacaacggggagatctaccgcaagctcaaagctgcctacgaccccgacggtcgactgctcgacctgtacaccaagtgcgtcggcggcgggtga

SEQ ID NO: 68 - protein, 470 aa, Thermobifida fusca
VNCQSSASNLANHINAVYELRRAYARLSADKPVRLAKTTSNLFRFRSRDDAARLDVSAFTSVISIDTEARVAEVGGMTTYEDLVAATLRHGLMPPVVPQLRTITLGGAVTGLGIESSSFRNGLPHESVEEMEILTGSGQVVVARRDNEHRDLFYGFPNSYGTLGYALRLRIQLEPVRPYVHLRHLRFTDAAAAMAALEQICADRTHDGETVDFVDGVVFARNELYLTLGTFTDRAPWTSDYTGTDIYYRSIPRYAGPGPGDYLTTHDYLWRWDTDWFWCSRAFGLQHPVVRRLWPRSLKRSDVYRKLVAWDRRTDASRLLDYYRGRPPKEPVIQDIEVEVGRAAEFLDFFHTEIGMSPVWLCPLRLREDTADDTEPVWPLYPLKPRRLYVNFGFWGLVPIRPGGGRTYHNRLIEKEVTRLGGHKSLYSDAFYDEDEFWELYNGEIYRKLKAAYDPDGRLLDLYTKCVGGG

SEQ ID NO: 69 - DNA, 1272 bp, Thermobifida fusca
  source 1..1272: mol_type = genomic DNA
atgcgactggcggaggtattcgaacgtgtcgtcggacccgatgcgcccgtccacttccgggcctacgacggcagcactgcgggagatccacgcagtgaagtcgctatcgtggttcgccacccggcagccgtcaactacatcgtccaagcgccgggagcactcggtttgacccgcgcctacgtggcgggatacctcgacgtcgaaggggacatgtacaccgcgctgcgggcaatggccgacgtggtgttccaggaccggccgcggctgtcccccggggaactgctgcggatcatccgcgggatcgggtgggtgaagttcgtcaaccggcttccaccgccgccgcaggaggtgcgccagtcccgcctcgccgccctgggctggcgccactccaagcagcgcgacgccgaagccatccagcaccactacgacgtctccaacgccttctacgccctggtcttgggcgagtcgatgacctacacctgcgcggtctacccgaccgagcaggccacgctggagcaggcacagttcttcaagcacgagctgatcgcccgcaagctcggtcttgcccctgggatacgactgctggatgtggggtgcggctggggcggcatggtcatccacgcggcccgggagcacggggtcaaagccctgggggtgaccctgtccaaagagcaggctgagtgggcgcagaagcggatcgcccacgagggcctgggcgacctggcagaagtccggcacatggactaccgggacctgcccgacggcgagtacgacgcgatcagctcgatcgggttgaccgagcacgtcggcaaaaagaacgtgcccgcctacttcgcgtcgctgtaccgcaagctcgtcccgggaggccgcctgctcaaccactgcatcacccggccccgcaacgacctgccgcccttcaaacgcggcggggtgatcaaccgctacgtcttccccgatggggagctggaagggcccggctggctgcaggcggcgatgaacgacgccgggttcgaaatccgccaccaggagaacctgcgggagcactacgcacggaccctgcgggactggctggccaacctggaccgcaactgggatgccgcggtgcgggaagtgggggagggcacggcccgagtgtggcggctctacatggccgggtgcgtgctcggcttcgaacgcaacgtggtgcaactgcaccagatcctcggggtgaagctcgacgggaccgaggcgcggatgccgctgcgccccgacttcgaaccgccgctgccttaa

SEQ ID NO: 70 - protein, 423 aa, Thermobifida fusca
MRLAEVFERVVGPDAPVHFRAYDGSTAGDPRSEVAIVVRHPAAVNYIVQAPGALGLTRAYVAGYLDVEGDMYTALRAMADVVFQDRPRLSPGELLRIIRGIGWVKFVNRLPPPPQEVRQSRLAALGWRHSKQRDAEAIQHHYDVSNAFYALVLGESMTYTCAVYPTEQATLEQAQFFKHELIARKLGLAPGIRLLDVGCGWGGMVIHAAREHGVKALGVTLSKEQAEWAQKRIAHEGLGDLAEVRHMDYRDLPDGEYDAISSIGLTEHVGKKNVPAYFASLYRKLVPGGRLLNHCITRPRNDLPPFKRGGVINRYVFPDGELEGPGWLQAAMNDAGFEIRHQENLREHYARTLRDWLANLDRNWDAAVREVGEGTARVWRLYMAGCVLGFERNVVQLHQILGVKLDGTEARMPLRPDFEPPLP

SEQ ID NO: 71 - DNA, 447 bp, Thermobifida fusca
  source 1..447: mol_type = genomic DNA
atggctgcgaccgatgacgaccggcaccacaccaccgtcgccctcgacctcatcgacgcgtatgtgcgcgccgaccgcagaatgatcggtgaacgttccgcggggatcagcgcggaggcgggggagcggatcgtctccaccctgaaagtgtgcgcggccttccttgcccgccgggtccaggagaccggggtgccgtggcgcgccgcggactcccgggaagcggtcgcccgcaccgtcgccgacctgctggaacccgaggtggaattcgcggtcgtctccgcctgggaggcgtacgcgatcggggagcacgaggccgcctgggtccgggcgcacggcgatccgctggtcttcgtccacatgctggccgcgttctccgctgctatcggcacagcggtctacggccgtgaggagctgctgcccacgctgcgcagggtgacagcacgataa

SEQ ID NO: 72 - protein, 148 aa, Thermobifida fusca
MAATDDDRHHTTVALDLIDAYVRADRRMIGERSAGISAEAGERIVSTLKVCAAFLARRVQETGVPWRAADSREAVARTVADLLEPEVEFAVVSAWEAYAIGEHEAAWVRAHGDPLVFVHMLAAFSAAIGTAVYGREELLPTLRRVTAR

SEQ ID NO: 73 - DNA, 1368 bp, Thermomonospora curvata
  source 1..1368: mol_type = genomic DNA
atgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctga

SEQ ID NO: 74 - protein, 455 aa, Thermomonospora curvata
MSQLAVTDHHERAVEALRRSYAAIPPGTPVRLAKQTSNLFRFREPTAAPGLDVSGFNRVLAVDPDARTADVQGMTTYEDLVDATLPHGLMPLVVPQLKTITLGGAVTGLGIESTSFRNGLPHESVLEMQIITGAGEVVTATPDGEHSDLFWGFPNSYGTLGYALKLKIELEPVKPYVRLRHLRFDDAGECAAKLAELSESREHEGDEVHFLDGTFFGPREMYLTLGTFTDTAPYVSDYTGQHIYYRSIQQRSIDFLTIRDYLWRWDTDWFWCSRALGVQNPLIRRVWPKSAKRSDVYRKLVAYEKRYQFKARIDRWTGKPPREDVIQDIEVPAERLPEFLEFFHDKIGMSPVWLCPLRARHRWPLYPLKPGVTYVNAGFWGTVPLQPGQMPEYHNRLIERKVAQLDGHKSLYSTAFYSREEFWRHYDGETYRRLKDTYDPDARLLDLYDKCVRGR

SEQ ID NO: 75 - DNA, 1263 bp, Thermomonospora curvata
  source 1..1263: mol_type = genomic DNA
atgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtga

SEQ ID NO: 76 - protein, 420 aa, Thermomonospora curvata
MTLAKVFEELVGADAPVELTAYDGSRAGRLGSDLRVHVKSPYAVSYLVHSPSALGLARAYVAGHLDAYGDMYTLLREMTQLTEALTPKARLRLLAGVLQDPLLRAAASRRLPPPPQEVRTGRTSWFRHTKRRDAKAISHHYDVSNTFYEWVLGPSMTYTCACFPTEDATLEEAQFHKHDLVAKKLGLRPGMRLLDVGCGWGGMVMHAAKHYGVRALGVTLSKQQAEWAQKAIAEAGLSDLAEVRHQDYRDVTEGDFDAISSIGLTEHIGKANLPSYFGFLYGKLKPGGRLLNHCITRPDNTQPAMKKDGFINRYVFPDGELEGPGYLQTQMNDAGFEIRHQENLREHYARTLAGWCRNLDEHWDEAVAEVGEGTARVWRLYMAGSRLGFELNWIQLHQILGVKLGERGESRMPLRPDWGV

SEQ ID NO: 77 - DNA, 7102 bp, synthetic construct
  source 1..7102: mol_type = other DNA
gtttgtggaagcggtattcgcaatttaattaaagctggtgacaattaatcatcggctcgtataatgtgtggaattgaatcgatataaggaggttaatcatgtgtctgtggttactactgacgcacaggctgcccatgccgccggcgtctcgcgtcttctggccagctaccgggcgatcccgcccagcgcgacagtgcgccttgcgaaaccgacgtccaacctgttccgcgcccgcgcccgcaccaatgtgaagggtctcgacgtctcgggcctgaccggtgtgatcggtgtcgacccggacgcgcgcaccgccgatgtggcgggcatgtgcacctacgaggacctggtggcggccacgcttccgtacggccttgccccactggtggtgccgcagctcaagaccatcacgctcggtggcgcggtcaccggtctgggcatcgagtccacgtcgttccgcaacggtctgccgcacgaaagtgtcctggagatggacatcttgaccggttcgggcgagatcgtcacggcctcaccggatcagcactcggatctgttccatgcgttccccaattcatatggaacccttggttattccacccggctgcgcatcgaactggagcccgtgcacccgtttgtggcgttgcgccacctgcgctttcactcgatcaccgatctggtcgcggcgatggaccggatcatcgagaccggcgggctggacggtgaacccgtcgactacctcgacggcgtggtgttcagcgcgactgagagttacctgtgtgttggcttcaagacgaaaacgccggggccggtcagcgattacacaggtcagcagatcttctaccggtcgatccagcatgacggcgacaccggcgccgagaaacacgaccggctgaccatccacgactacctgtggcgctgggacaccgactggttctggtgctcacgggcattcggcgctcagcatccggtgatccgcaggttctggccgcggcggctgcgccgcagcagcttctactggaagctggtggcctacgaccagcggtacgacatcgccgaccgtatcgagaagcgcaacgggcgcccgccgcgcgagcgggtggtccaggacgtcgaggtgcccatcgagcggtgcgcggacttcgtcgagtggttcctgcagaatgtgccgatcgagccgatctggctgtgccccctacggttgcgtgacagcgccgacggcggtgcctcgtggcccctgtatccgctgaaggcgcaccacacctacgtcaacatcggtttctggtcatcagtgccggtgggccccgaggagggccacaccaaccgcctcatcgagaaaaaagtcgcggagctggacgggcacaaatctttgtactcggacgcttattacacacgtgacgaattcgacgagctgtacggcggtgaggtctacaacaccgtcaagaagacgtacgacccggattcacgtctgctagacctgtattcgaaggcggtgcaaagacaatgaccacattcaaagaacgcgagacgtccacagcggaccgcaagctcaccctggccgagatcctcgagatcttcgccgcgggtaaggagccgctgaagttcactgcgtacgacggcagctcggccggtcccgaggacgccacgatgggtctggacctcaagaccccgcgtgggaccacctatctggccacggcacccggcgatctgggcctggcccgtgcgtatgtctccggtgacctggagccgcacggcgtgcatcccggcgatccctacccgctgctgcgcgccctggccgaacgcatggagttcaagcgcccgcctgcgcgtgtgctggcgaacatcgtgcgctccatcggcatcgagcacctcaagccgatcgcaccgccgccgcaggaggcgctgccccggtggcgccgcatcatggagggcctgcggcacagcaagacccgcgacgccgaggccatccaccaccactacgacgtgtcgaacacgttctacgagtgggtgctgggcccgtcgatgacctacacgtgcgcgtgctaccccaccgaggacgcgaccctcgaagaggcccaggacaacaagtaccgcctggtgttcgagaagctgcgcctgaagcccggtgaccggttgctcgacgtgggctgcggctggggcggcatggtccgctacgcggcccgccacggcgtcaaggcgctcggtgtcacgctcagccgcgaacaggcgacgtgggcgcagaaggccatcgcccaggaaggtctcaccgatctggccgaggtgcgtcacggtgattaccgcgacgtcatcgaatccgggttcgacgcggtgtcctcgatcgggctgaccgagcacatcggcgtgcacaactacccggcgtacttcaacttcctcaagtcgaagctgcgcaccggtggcctgctgctcaaccactgcatcacccgcccggacaaccggtcggcgccatcggccggcgggttcatcgacaggtacgtgttccccgacggggagctcaccggctcgggccgcatcatcaccgaggcccaggacgtgggccttgaggtgatccacgaggagaacctacgcaatcactatgcgatgacgctgcgcgactggtgccgcaacctggtcgagcactgggacgaggcggtcgaagaggtcgggctgcccaccgcgaaggtgtggggcctgtacatggccggctcacgtctgggcttcgagaccaatgtggttcagctgcaccaggttctggcggtcaagcttgacgatcagggcaaggacggcggactgccgttgcggccctggtggtccgcctagcctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 78 - DNA, 10766 bp, synthetic construct
  source 1..10766: mol_type = other DNA
ttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgtttaaactgcgtcggaacgggatatgcattcccctagtttcgccgcagtgcagaatcaggcggtttctttgcaccacaccacatacggaggatgacgggcattattgatgttgaatagtaacctgatcgtgactagtatgacggaacccaacagcaacagccgaccgtttgtgagcgtttttgcggccggtcaggcgagtttttccggcctgccaatggtccttccgtaccctttaccctgtacgctgtacctgccacggataggccgtgctccacctgctcactatggtgggtgcggggaaaacaacaggcaggctcaattgctctgcaaatgggttgagggggtgattgatgtcactggtacaccaacaggggaatgctcggcgttgattttgggccacctcttttgtttgccagagcttgtctctattgtcaaatttaacggtctgcaactgttgcccaaaatgggacaatgatccgatgcctgcatagacaccctgcttgagggtgcgatcgccctaatacgaggcaaaccaagttttccaattgaccttcaattgacgagcggttgttgcgacaggggactggagtgctacctgtttagagttcaaatccgtcacccagcattgaaagtttttccccgcattggatgattgcaatgccgctaacccgctcatccgccaaagttcatagtcccaccctgcctcgacttatcggaccacatggggctcccttatgcgcgcgcatatggcgcttgattgctttttggtcaacgtttgggacaaatttcctttgttaaggcggacccgccagcagatacgaaggtataaatagggctcactttcaccatcttgtccattcaattgcaagactcaaaagtaataatgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggagacctggccggctttgtggtcgtttcttactccggatggaaccgacgactgaccgtggaggacatcgaggtcgctcctgagcaccgaggtcatggtgtcggacgagctctgatgggtctcgctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcagagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatgtccgttgttaccaccgatgctcaagctgctcatgctgctggtgtttctagattattggcttcttatagagccattccaccatctgctactgttagattggctaagccaacttctaatttgttcagagctagagctagaactaacgttaagggtttggatgtttctggtttgactggtgttattggtgttgatccagatgctagaactgctgatgttgctggtatgtgtacttacgaagatttggttgctgctactttgccatatggtttggctccattggttgttccacaattgaaaactattactttgggtggtgctgttaccggtttgggtattgaatctacttctttcagaaacggtttgccacacgaatctgttttggaaatggatattttgaccggttccggtgaaatagttactgcttctccagatcaacactccgatttgtttcatgcttttccaaactcttacggtacattgggttactctaccagattgagaattgaattggaaccagttcatccattcgttgccttgagacatttgagattccattccattactgatttggtcgcagccatggatagaattattgaaactggtggtttagacggtgaaccagttgattatttggatggtgttgttttctctgccaccgaatcatatttgtgtgttggtttcaaaactaagaccccaggtccagtttctgattatactggtcaacaaatcttctacagatccatccaacatgatggtgatactggtgctgaaaaacatgatagattgaccatccatgactacttgtggagatgggatactgattggttttggtgttctagagcttttggtgctcaacatccagttattagaagattctggccaagaagattaagaagatcctccttctactggaaattggttgcttacgatcaaagatacgatatcgccgatagaatcgaaaagagaaatggtagaccaccaagagaaagagttgttcaagacgttgaagttccaattgaaagatgcgctgatttcgttgaatggttcttgcaaaatgttccaatcgaacctatttggttgtgcccattgagattgagagattctgctgatggtggtgcttcatggccattatatccattgaaagctcatcacacctacgtcaatattggtttctggtcatctgttccagttggtccagaagaaggtcataccaatagattgattgaaaaaaaggtcgccgaattggacggtcacaaatcattatattctgatgcctactacaccagagatgaattcgatgaattatacggtggtgaagtttacaacaccgtcaaaaaaacttacgacccagactcaagattattagacttgtactctaaggccgtccaaagacaatgagctgcttgtacctagtgcaaccccagtttgttaaaaattagtagtcaaaaacttctgagttagaaatttgtgagtgtagtgagattgtagagtatcatgtgtgtccgtaagtgaagtgttattgactcttagttagtttatctagtactcgtttagttgacactgatctagtattttacgaggcgtatgactttagccaagtgttgtacttagtcttctctccaaacatgagagggctctgtcactcagtcggcctatgggtgagatggcttggtgagatctttcgatagtctcgtcaagatggtaggatgatgggggaatacattactgctctcgtcaaggaaaccacaatcagatcacaccatcctccatggtatccgatgactctcttctccacagtcgcagtaggatgtcctgcacgggtctttttgtggggtgtggagaaaggggtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacgtgatagttgggggtgtgcgtgttaaaggaaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcggcagggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacaatgaccaccttcaaagaaagagaaacttctaccgctgatagaaagttgaccttggctgaaattttggaaattttcgctgctggtaaagaaccattgaagttcactgcttatgatggttcttctgctggtcctgaagatgctactatgggtttggatttgaaaactccaagaggtactacttacttggctactgctccaggtgatttgggtttggctagagcttatgtttctggtgacttggaaccacatggtgttcatcctggtgatccatatccattattgagagctttagccgaaagaatggaattcaaaagaccaccagctagagttttggctaacatcgttagatccattggtatcgaacatttgaagccaattgctccaccaccacaagaagctttgccaagatggagaagaattatggaaggtttgagacactctaagaccagagatgctgaagctattcatcatcactacgatgtttctaacaccttctacgaatgggttttgggtccatctatgacttatacttgtgcttgttacccaacagaagatgccactttggaagaagctcaagataacaagtacagattggtctttgaaaagttgagattgaagccaggtgacagattattggatgttggttgtggttggggtggtatggttagatatgctgctagacatggtgtaaaagctttgggtgttactttgtctagagaacaagctacttgggctcaaaaagctattgctcaagaaggtttaaccgatttggctgaagttagacacggtgattacagagatgttatcgaatctggtttcgatgccgtttcttctattggtttgactgaacatatcggtgttcataactatccagcctacttcaacttcttgaagtctaagttgagaaccggtggtttgttgttgaaccattgcattactagaccagataacagatctgctccatctgctggtggttttattgatagatacgttttcccagatggtgaattgactggttccggtagaattattactgaagcacaagatgtcggtttggaagttatccatgaagaaaacttgagaaaccattacgccatgactttgagagattggtgtagaaacttggttgaacattgggatgaagccgttgaagaagttggtttgccaactgctaaagtttggggtttgtatatggctggttctagattaggttttgaaactaacgttgtccaattgcaccaagttttggcagttaagttggatgatcaaggtaaagatggtggtttgcctttaagaccatggtggtctgcttgagcattagcgactactaatatatatttgaatccatggaattataacaaacaagcatcaaaacaagaattagcgacattatacttgaaatcagcattagcgatactactaatatagtttattctatgtaatgatccatggaagttcgattgatttgccaagttaatttgatagattatgcatgccatttagtcgacgcaggtacgatctacagcgataaagaagaggttgtgggtcattcaattttgcaccaattttgcaccatcatagatcataatacatttacaaggcctacaattcttacagggtcttctcgagagcaattccttaattaaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagc

SEQ ID NO: 79 - DNA, 10970 bp, synthetic construct
  source 1..10970: mol_type = other DNA
ttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgtttaaactgcgtcggaacgggatatgcattcccctagtttcgccgcagtgcagaatcaggcggtttctttgcaccacaccacatacggaggatgacgggcattattgatgttgaatagtaacctgatcgtgactagtatgacggaacccaacagcaacagccgaccgtttgtgagcgtttttgcggccggtcaggcgagtttttccggcctgccaatggtccttccgtaccctttaccctgtacgctgtacctgccacggataggccgtgctccacctgctcactatggtgggtgcggggaaaacaacaggcaggctcaattgctctgcaaatgggttgagggggtgattgatgtcactggtacaccaacaggggaatgctcggcgttgattttgggccacctcttttgtttgccagagcttgtctctattgtcaaatttaacggtctgcaactgttgcccaaaatgggacaatgatccgatgcctgcatagacaccctgcttgagggtgcgatcgccctaatacgaggcaaaccaagttttccaattgaccttcaattgacgagcggttgttgcgacaggggactggagtgctacctgtttagagttcaaatccgtcacccagcattgaaagtttttccccgcattggatgattgcaatgccgctaacccgctcatccgccaaagttcatagtcccaccctgcctcgacttatcggaccacatggggctcccttatgcgcgcgcatatggcgcttgattgctttttggtcaacgtttgggacaaatttcctttgttaaggcggacccgccagcagatacgaaggtataaatagggctcactttcaccatcttgtccattcaattgcaagactcaaaagtaataatgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggagacctggccggctttgtggtcgtttcttactccggatggaaccgacgactgaccgtggaggacatcgaggtcgctcctgagcaccgaggtcatggtgtcggacgagctctgatgggtctcgctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcagagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatgaagttctctatgccatcttggggtgttgttttttacgctttgttggtttgtttgttgccattcttgtctaaggctggtgttcaagctatgtccgttgttaccaccgatgctcaagctgctcatgctgctggtgtttctagattattggcttcttatagagccattccaccatctgctactgttagattggctaagccaacttctaatttgttcagagctagagctagaactaacgttaagggtttggatgtttctggtttgactggtgttattggtgttgatccagatgctagaactgctgatgttgctggtatgtgtacttacgaagatttggttgctgctactttgccatatggtttggctccattggttgttccacaattgaaaactattactttgggtggtgctgttaccggtttgggtattgaatctacttctttcagaaacggtttgccacacgaatctgttttggaaatggatattttgaccggttccggtgaaatagttactgcttctccagatcaacactccgatttgtttcatgcttttccaaactcttacggtacattgggttactctaccagattgagaattgaattggaaccagttcatccattcgttgccttgagacatttgagattccattccattactgatttggtcgcagccatggatagaattattgaaactggtggtttagacggtgaaccagttgattatttggatggtgttgttttctctgccaccgaatcatatttgtgtgttggtttcaaaactaagaccccaggtccagtttctgattatactggtcaacaaatcttctacagatccatccaacatgatggtgatactggtgctgaaaaacatgatagattgaccatccatgactacttgtggagatgggatactgattggttttggtgttctagagcttttggtgctcaacatccagttattagaagattctggccaagaagattaagaagatcctccttctactggaaattggttgcttacgatcaaagatacgatatcgccgatagaatcgaaaagagaaatggtagaccaccaagagaaagagttgttcaagacgttgaagttccaattgaaagatgcgctgatttcgttgaatggttcttgcaaaatgttccaatcgaacctatttggttgtgcccattgagattgagagattctgctgatggtggtgcttcatggccattatatccattgaaagctcatcacacctacgtcaatattggtttctggtcatctgttccagttggtccagaagaaggtcataccaatagattgattgaaaaaaaggtcgccgaattggacggtcacaaatcattatattctgatgcctactacaccagagatgaattcgatgaattatacggtggtgaagtttacaacaccgtcaaaaaaacttacgacccagactcaagattattagacttgtactctaaggccgtccaaagacaacatgatgaattgtgagctgcttgtacctagtgcaaccccagtttgttaaaaattagtagtcaaaaacttctgagttagaaatttgtgagtgtagtgagattgtagagtatcatgtgtgtccgtaagtgaagtgttattgactcttagttagtttatctagtactcgtttagttgacactgatctagtattttacgaggcgtatgactttagccaagtgttgtacttagtcttctctccaaacatgagagggctctgtcactcagtcggcctatgggtgagatggcttggtgagatctttcgatagtctcgtcaagatggtaggatgatgggggaatacattactgctctcgtcaaggaaaccacaatcagatcacaccatcctccatggtatccgatgactctcttctccacagtcgcagtaggatgtcctgcacgggtctttttgtggggtgtggagaaaggggtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacgtgatagttgggggtgtgcgtgttaaaggaaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcggcagggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacaatgaagttctctatgccatcttggggtgttgttttttacgctttgttggtttgtttgttgccattcttgtctaaggctggtgttcaagctatgaccaccttcaaagaaagagaaacttctaccgctgatagaaagttgaccttggctgaaattttggaaattttcgctgctggtaaagaaccattgaagttcactgcttatgatggttcttctgctggtcctgaagatgctactatgggtttggatttgaaaactccaagaggtactacttacttggctactgctccaggtgatttgggtttggctagagcttatgtttctggtgacttggaaccacatggtgttcatcctggtgatccatatccattattgagagctttagccgaaagaatggaattcaaaagaccaccagctagagttttggctaacatcgttagatccattggtatcgaacatttgaagccaattgctccaccaccacaagaagctttgccaagatggagaagaattatggaaggtttgagacactctaagaccagagatgctgaagctattcatcatcactacgatgtttctaacaccttctacgaatgggttttgggtccatctatgacttatacttgtgcttgttacccaacagaagatgccactttggaagaagctcaagataacaagtacagattggtctttgaaaagttgagattgaagccaggtgacagattattggatgttggttgtggttggggtggtatggttagatatgctgctagacatggtgtaaaagctttgggtgttactttgtctagagaacaagctacttgggctcaaaaagctattgctcaagaaggtttaaccgatttggctgaagttagacacggtgattacagagatgttatcgaatctggtttcgatgccgtttcttctattggtttgactgaacatatcggtgttcataactatccagcctacttcaacttcttgaagtctaagttgagaaccggtggtttgttgttgaaccattgcattactagaccagataacagatctgctccatctgctggtggttttattgatagatacgttttcccagatggtgaattgactggttccggtagaattattactgaagcacaagatgtcggtttggaagttatccatgaagaaaacttgagaaaccattacgccatgactttgagagattggtgtagaaacttggttgaacattgggatgaagccgttgaagaagttggtttgccaactgctaaagtttggggtttgtatatggctggttctagattaggttttgaaactaacgttgtccaattgcaccaagttttggcagttaagttggatgatcaaggtaaagatggtggtttgcctttaagaccatggtggtctgctcatgatgaattgtgagcattagcgactactaatatatatttgaatccatggaattataacaaacaagcatcaaaacaagaattagcgacattatacttgaaatcagcattagcgatactactaatatagtttattctatgtaatgatccatggaagttcgattgatttgccaagttaatttgatagattatgcatgccatttagtcgacgcaggtacgatctacagcgataaagaagaggttgtgggtcattcaattttgcaccaattttgcaccatcatagatcataatacatttacaaggcctacaattcttacagggtcttctcgagagcaattccttaattaaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagc

SEQ ID NO: 80 - DNA, 1410 bp, synthetic construct
  source 1..1410: mol_type = other DNA
atgtccgttgttaccaccgatgctcaagctgctcatgctgctggtgtttctagattattggcttcttatagagccattccaccatctgctactgttagattggctaagccaacttctaatttgttcagagctagagctagaactaacgttaagggtttggatgtttctggtttgactggtgttattggtgttgatccagatgctagaactgctgatgttgctggtatgtgtacttacgaagatttggttgctgctactttgccatatggtttggctccattggttgttccacaattgaaaactattactttgggtggtgctgttaccggtttgggtattgaatctacttctttcagaaacggtttgccacacgaatctgttttggaaatggatattttgaccggttccggtgaaatagttactgcttctccagatcaacactccgatttgtttcatgcttttccaaactcttacggtacattgggttactctaccagattgagaattgaattggaaccagttcatccattcgttgccttgagacatttgagattccattccattactgatttggtcgcagccatggatagaattattgaaactggtggtttagacggtgaaccagttgattatttggatggtgttgttttctctgccaccgaatcatatttgtgtgttggtttcaaaactaagaccccaggtccagtttctgattatactggtcaacaaatcttctacagatccatccaacatgatggtgatactggtgctgaaaaacatgatagattgaccatccatgactacttgtggagatgggatactgattggttttggtgttctagagcttttggtgctcaacatccagttattagaagattctggccaagaagattaagaagatcctccttctactggaaattggttgcttacgatcaaagatacgatatcgccgatagaatcgaaaagagaaatggtagaccaccaagagaaagagttgttcaagacgttgaagttccaattgaaagatgcgctgatttcgttgaatggttcttgcaaaatgttccaatcgaacctatttggttgtgcccattgagattgagagattctgctgatggtggtgcttcatggccattatatccattgaaagctcatcacacctacgtcaatattggtttctggtcatctgttccagttggtccagaagaaggtcataccaatagattgattgaaaaaaaggtcgccgaattggacggtcacaaatcattatattctgatgcctactacaccagagatgaattcgatgaattatacggtggtgaagtttacaacaccgtcaaaaaaacttacgacccagactcaagattattagacttgtactctaaggccgtccaaagacaacatgatgaattg

SEQ ID NO: 81 - DNA, 1311 bp, synthetic construct
  source 1..1311: mol_type = other DNA
atgaccaccttcaaagaaagagaaacttctaccgctgatagaaagttgaccttggctgaaattttggaaattttcgctgctggtaaagaaccattgaagttcactgcttatgatggttcttctgctggtcctgaagatgctactatgggtttggatttgaaaactccaagaggtactacttacttggctactgctccaggtgatttgggtttggctagagcttatgtttctggtgacttggaaccacatggtgttcatcctggtgatccatatccattattgagagctttagccgaaagaatggaattcaaaagaccaccagctagagttttggctaacatcgttagatccattggtatcgaacatttgaagccaattgctccaccaccacaagaagctttgccaagatggagaagaattatggaaggtttgagacactctaagaccagagatgctgaagctattcatcatcactacgatgtttctaacaccttctacgaatgggttttgggtccatctatgacttatacttgtgcttgttacccaacagaagatgccactttggaagaagctcaagataacaagtacagattggtctttgaaaagttgagattgaagccaggtgacagattattggatgttggttgtggttggggtggtatggttagatatgctgctagacatggtgtaaaagctttgggtgttactttgtctagagaacaagctacttgggctcaaaaagctattgctcaagaaggtttaaccgatttggctgaagttagacacggtgattacagagatgttatcgaatctggtttcgatgccgtttcttctattggtttgactgaacatatcggtgttcataactatccagcctacttcaacttcttgaagtctaagttgagaaccggtggtttgttgttgaaccattgcattactagaccagataacagatctgctccatctgctggtggttttattgatagatacgttttcccagatggtgaattgactggttccggtagaattattactgaagcacaagatgtcggtttggaagttatccatgaagaaaacttgagaaaccattacgccatgactttgagagattggtgtagaaacttggttgaacattgggatgaagccgttgaagaagttggtttgccaactgctaaagtttggggtttgtatatggctggttctagattaggttttgaaactaacgttgtccaattgcaccaagttttggcagttaagttggatgatcaaggtaaagatggtggtttgcctttaagaccatggtggtctgct

SEQ ID NO: 82 - DNA, 4399 bp, synthetic construct
  source 1..4399: mol_type = other DNA
tgggtaggttatatagggatatagcacagagatatatagcaaagagatacttttgagcaatgtttgtggaagcggtattcgcaatttaattaaagctggtgacaattaatcatcggctcgtataatgtgtggaattgaatcgatataaggaggttaatcatgtttaaaccctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtgga

SEQ ID NO: 83 - DNA, 7531 bp, synthetic construct
  source 1..7531: mol_type = other DNA
gtttgtggaagcggtattcgcaatttaattaaagctggtgacaattaatcatcggctcgtataatgtgtggaattgaatcgatataaggaggttaatcatgtgtctgttccttcgaccgacgcacgttctgctcacgccgacggcgtgcagcggcttctcgccagctatcgggcgattccccaagacgccacggtccggctggccaaacccacgtcgaacctcttccgtgcccgcgcgaaaaccaggaccaagggtctggacacgtctgggttgacgaacgtgatcgcggtcgacgcggaggcacgcaccgccgatgtggcagggatgtgcacctacgaagacctggtcgcggccacgctgccgcatggactttcgccgctggtggtgccgcagttgaagacgatcaccctcggcggggcggtcaccggactcgggatcgagtccgcctcgttccgcaacggcctgccacacgaatcggttctcgagatggacgtcctcaccggcaccggtgatgtcgtgcgcgcctcccccgacgagaaccctgacctgtttcgggcgtttccgaattcctatggcacgttgggctattcggttcggctcaagatcgagctggaaccggtgaagccgttcgtcgcgctgcgccacctccgtttccattcgctgtcggctctcatcgaggcgatggaccgcatcgtcgaaaccggcggcctcaacggcgaaccggtggactacctcgacggcgtcgtgttcagtgccgaggagagttacctgtgcgtggggcagcgctccgcgacaccgggcccggtcagcgactacacgggcaagcagatctactaccgctcgattcagcacgacggcccgaccgatggcgccgagaagcacgaccggctgaccatccacgactacctgtggcgctgggacaccgactggttctggtgctcaagggcattcggcgcgcagaacccgcggatccggcgctggtggccgcgccggtaccggcgcagcagtgtgtactggaagctgatcggctacgaccggcgtttcggtatcgccgatcgcatcgagaagcgcaacggccgacccccgcgcgagcgggtggtccaggacatcgaggtgcccatcgagcggaccgtcgagtttctgcagtggtttctcgacaccgtgcccatcgaaccgatctggttgtgcccgttgcggctccgcgacgaccgcgattggcccctgtatccgatccgaccccaccacacctacgtcaacgtgggtttctggtcgtcggtgccggtgggcccggaggagggctacaccaacaggatgatcgaacggaaagtcagcgacctcgacggtcacaaatcgctgtattccgatgcgtactactcgccggaagagtttgattcgctctatggcggggagacgtacaagacggtgaagaagacatacgacccagactctcgtttcctggacctgtacggcaaagcagtggggcggcaatgagcgttgacgcgaagaacggaggccacagttgacgacatttcgggacggcgcggccgacaccggcctgcacggagaccgcaagctcaccctggcggaggtcttggaggtcttcgcctcgggccgactgcctctgaagttcacggcgtacgacggcagcagcgcgggcccggacgacgccacgctcgggctggacctgctgaccccccgcgggaccacgtacctcgcaacggctcccggcgatctcggcctggcccgggcctacgtctccggtgacctgcagttgcagggggtgcaccctggcgacccgtacgacctgctcaacgcactggtgcagaaactggacttcaagcgaccgtccgcccgggtgctggcgcaggtcgtccgatcgatcgggatcgagcacctgaaaccgatcgcgccaccgccgcaggaggcgctgccgcggtggcggcgcatcgcagaaggactgcggcacagcaagacccgtgacgccgacgcgatccaccaccattacgatgtctccaacaccttctacgagtgggtgctcgggccgtcgatgacctacacctgcgcctgctacccgcatcccgacgccaccctcgaggaggcgcaggagaacaaatatcggctggtgttcgagaaactgcgcctcaagccgggcgaccgccttctcgacgtgggttgcgggtggggcggaatggtgcgctacgcggcccgtcacggcgtcaaggcgatcggggtgacgctgtccagggagcaggcgcagtgggcacgcgccgccatcgaacgggacggcctgggtgacctcgccgaggtccgccacagcgactaccgcgatgtgcgcgagtcccagttcgacgccgtgtcttcgctggggctcaccgagcacatcggggtcgccaactatccgtcgtacttccggttcctcaagtcgaagttgcgcccgggcggcctactgctcaaccactgcatcacccggcacaacaatcgcaccggccccgccgccgggggattcatcgaccggtatgtgttcccggacggggagctgaccggatcgggccggatcatcaccgagatccaggacgtcggtttggaggtgatgcacgaagagaacctgcgccggcactatgcgctgacacttcgggactggtgccggaatctggtgcagcactgggacgaagcggtcgcagaggtcggcctgcccaccgccaaggtgtggggtctgtacatggctgcctcgcgggtcggcttcgagcagaacagcattcagctgcatcaggtactggcggtgaagctcgacgaacgtggcggggacggcggtttgccgttgcggccctggtggaccgcgtagcaactatgctcaccgtgtgatccgctttctgctgcgcgtcgcggtctttctcggatcgtcggcgatcgggctactggtggccggctggctggtgccgggggtgtcgctgtcggtgctgggcttcgtcaccgcggtggtgatcttcacggtggcacaagggattctgtcgccgttcttcctgaagatggccagccgctacgcgtcggccttcctcggcggcatcggcctggtgtccacgttcgtggcgctgctgctcgcgtcgctgctgtccaacgggctcagcatccgcggcgtcgggtcgtggatcgcggccacggtggtggtctggctggtcacagccctggcgaccgtcgtgctgcccgttctggtgctgcgggagaagaagaaagcagcctgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 84 - DNA, 7126 bp, synthetic construct
  source 1..7126: mol_type = other DNA
gtttgtggaagcggtattcgcaatttaattaaagctggtgacaattaatcatcggctcgtataatgtgtggaattgaatcgatataaggaggttaatcatatgacgcctgaagctagtgcggcggcgcacgccgctgcggtggatcgcctcatccatagctatcgggcgattcctgatgacgcgccggtgcggctggcgaagaagacgtcaaacctattccgccacagggaaaagacttctgctcctgggcttgacgtatccggcctggctcgcgtgattgggatcgactcagacactcgcactgccgacgttggcggcatgtgcacatacgaggaccttgtcgcggcgacgctcgaatacgatctggtccccctggtcgtcccgcaactcaaaacgatcactctcggcggcgcggtgacgggcctgggaattgagtccacctcgttccgcaatgggcttccccatgaatctgttctcgaaatggatatcctgacgggcgccggggaggtcgtcacggccggcccggaaggcccccatagcgatttgtactgggggtttccgaattcgtacggcacgctcggctatgcgacgcgcctgcgcatcgaactagaaccggtcgagccgtacgtcgaactcaggcacctgcggttcactagcctcgatgagcttcaggagacacttgacaccgtttcgtacgaacacacgtatgacggggaacccgttcattacgtcgatggagtcatgttctcagccacggaaagctacctcacgcttggccgtcagacgagcgaacccggcccggtcagcgactacaccggaaaccagatctactaccgttcaatacagcacggtggcgctgaaactcccgtcgtcgaccggatgaccattcatgactatctatggcgctgggatactgactggttctggtgctcgcgtgccttcggaacgcaacacccagtggtccggagattctggccacgccgctatcgccgcagcagcttctactggaagctgatcgcgcttgaccgccaggttgggctcgcggacttcatcgaacaacggaagggcaacctcccccgggaacgcgtagtccaggacatcgaggtcccgatcgagaacactgcgagcttcttgcggtggttcttggcgaacgtgccgatcgagccggtatggctatgcccgctgcgcctgcgaaaaacacgcagccccggcctgccttcgccgacgtccccggcttcacgcccatggcccctctatccgctcgagcctcagcgcacatacgtcaatgttggcttctggtcagcggtgccggtcgtggccggccagcccgaggggcacaccaaccggatgatcgagaacgaagtcgatcgccttgacggtcacaaatcgctgtactcagatgcgttttacgagcgaaaagagtttgacgcgctgtacggcggcgatacctatagagaactcaaagagacctacgacccaaacagccggttacttgatctctatgcaaaggcggtgcaaggacgatgaaggcagtgttgacggcgtttacggctccccaactcgaaaggatgaacgtcgctgagatactcagcgcggtactcgggcgagatttcccgatccggttcactgcgtacgacggcagcgcgctcggccccgaaaccgcccgctacggcttgcacctcacgacgccgcgcgggctgacctacctcgctaccgcgcccggtgatctcgggctcgcacgcgcgtacgtgtccggcgacctcgaggtcagtggggttcatcagggtgacccgtacgagataatgaagatcctcgcgcatgacgtccgggtgcggcggccctcgccagcaacgatcgcttcgatcatgcggtccctcggctgggaacgcttgcgaccggtcgcgccgcccccgcaagagaacatgccccgttggcgccggatggcccttggcctgctgcactcgaagagccgtgatgctgcggcaatccaccatcattacgacgtgtcgaacgagttttacgagcacatcctcggcccgtcgatgacgtacacatgcgcggcctaccccagcgcagacagttccctggaggaagcacaggacaacaagtaccgactcgtcttcgagaaacttggcctgaaagccggggatcgcctgcttgacgtcgggtgcgggtggggcggcatggtgcggttcgccgctaagcgcggcgttcatgtcatcggtgcgacattgtcccgcaaacaggcggaatgggctcagaagatgattgcccatgaaggattgggcgatctggcggaagtccgtttctgcgactaccgcgatgtcacagaggcgggcttcgacgcagtgtcgtcgatcggcctcactgaacacatcggtttggcgaactacccgtcgtacttcggcttcctgaaggacaagttgcggccaggcggacgactgctgaaccattgcatcactcgcccgaacaaccttcaaagcaaccgcgcaggtgacttcattgaccggtacgttttccctgacggagagctcgccggacctggcttcatcatttcagctgtccacgacgccggtttcgaggtgcggcacgaagagaacctccgcgagcactacgcactgacgctgcgggactggaaccgcaacctcgctcgcgactgggacgcgtgtgtgcacgcctccgacgagggcaccgcccgcgtctggggactgtacatttccggttcacgagtcgcgtttgaaacgaactcgattcagctgcaccaggtcctggcggtcaaaaccgcgcggaatggcgaagcgcaggtcccgttgggtcagtggtggacccgctgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 85 - DNA, 7925 bp, synthetic construct
  source 1..7925: mol_type = other DNA
gtttgtggaagcggtattcgcaatttaattaaagctggtgacaattaatcatcggctcgtataatgtgtggaattgaatcgatataaggaggttaatcatgtgaccgtcgccggcaggatcactgacgcggtacgcataggaaatggacttgaccagcgagatctagcccccgtcgggtggtacgcacacgaacaggccgtggcgcgactgaaggccagtttcgacgcggtccccgccgggcgtcgcgtgcggctggcgaagaagacgtccaaccttttccgcgggcgttccggcgaggcagtcgggctcgacgtgtcggggctgcacggcgtcatcgccgtcgaccccgttgagggcaccgctgacgtccagggcatgtgcacgtacgaggacctggtggacgtcctgctgccctacggtctggcgcccaccgtcgttccgcagctgaagaccatcactctcggcggtgcggtgaccggcatgggggtggaatccacctccttccgcaacggcctgccgcacgaagccgtcctggaaatggatgtgctcaccggtaccggagacatcctcacctgttcgccgacccagaacaccgacctctaccgcggcttccccaactcctacggttccctgggatacagcgtgcggctgaaggtgcggtgcgaacgggtggaaccctacgtcgacctgcggcatgtacgcttcgatgacgttcagtcgctcaccgacgccctcgacaacatcgtcgtggacaaggagtacgagggtgaacgggtcgactatctcgacggtgtggtcttcagcctggaggagagctacctcgtcctgggacgggcgaccagcgaggccggccccgttagcgactacacccgcgagcgcagttactaccgttctctgcagcatccgtcgggggtcctgcgcgacaagttgaccatccgcgactacctctggcggtgggacgtcgactggttctggtgcaaccgggccttcggtacccagaaccccaccatccgtactctgtggccgcgggatctcctgcggtcgagcttctactggaagatcatcggctgggaccgacgcttcgacatcgcggaccggatcgaggcacacaacgggcgccccgcacgcgagcgcgtcgtccaggacatcgaggtcacccccgacaacctgccggagttcctcacgtggttcttcacccactgcgagatcgagccggtgtggctgtgccccattcgactggccgacgactcgggcgagcggacaccgtggcccctgtacccgctgtcacccggcgacacctgggtcaacgtgggattctggagctcggtgcccgccgacctgatggggaaggacgccccgaccggagccttcaaccgggaggtggagagagtcgtctcggacctcggcggacacaagtcgttgtactccgaggcattctattctgaggaacagttcgccgccctctacggcggtgaacgtcccgcacaactcaaggcggtcttcgacccggatgaccggttccccgggttgtacgagaagaccgtgggcggcgtctgacgacacgcacgacgacgcacaccgagcacgatgacgcacgacaagcacgatgacgcatgatgaccaagaggagagagatgagcaggggattcacgccgctgacggtgggacagatcgtggacaaggtcatcacaccgccggcaccgttccgggtgaccgctttcgacggatccaccgcggggccggcagacgcggaactggcactggagatcacatcgccggacgccctggcctatatcgtgaccgcgccgggcgacctcggactggcacgcgcctacatcaccggaagcctccgcgtcaccggtgacgagcccggccacccgtacctcgtctttgaccacctccagcacctttacgaccagatccgacgcccctcggcgaaggacctgctggatatcgcccgctcgctgaaggccatgggggcgatcaaggtgcagccggcaccggagcaggagaccctcccgggctggaagagggccatactcgaggggctgtcccggcactctccggaacgggacaaggaggtcgtgagccgccactacgacgtgggcaatgacttctacgagctcttcctcggcgattccatggcctacacctgtgcctactatcccgagtttgacggtgagaaccaggtcaccggtcccaccggcgggtggcggtacgacgactgggagaaagggccgaccgccaacgggccgttgacccaggcgcaggacaacaagcatcgcctggtcttcgacaagctgcgactcaacccgggtgaccggttgttggacgtcggctgcgggtggggcggtatggtgcggtacgccgcccgccacggcgtgaaggccatcggtgtcacgctgtcccgagagcagtacgagtggggtaaggcgaagatcgaggaggagggtctgcaggacctcgccgaggtccggtgtatggactaccgtgacgtgccggagtccgacttcgacgcggtcagtgccatcggcatcctcgagcacatcggcgtgcccaactacgaggactacttcacccgcctgttcgccaagctgcgcccgggcggtcggatgctgaaccactgcatcacccgtccgcacaaccggaagacgaagaccggccagttcatcgaccgctacatcttccccgacggtgagctgaccggctcgggccggatcatcacgatcatgcaggacaccggattcgacgtcgtccacgaggagaatctgcgaccgcactaccagcgcacgttgcatgactggtgtgaactgttggccaccaactgggaccaggccgtccatctcgtgggcgaggagacggctcgtctgttcggcctgtacatggcggggtcggaatggggtttcgaacacaacgtgatccagctccaccaggttctcggcgtgaagccggacgcggcaggcagttccggggtgccggtccgccagtggtggaggtcctgacggtaacgtcgggacgatgagacggatcaccagaggcgctgcggtggcggtgctgtgcacaccgttgctgctcggagcctgcaccatcggcgacgcgggaccgggggacgagaccacggaccctgtcgtggacactgaagcaccgcccgataaaccggtgccggactctgcggcggaatccggcgctgaagacggacctgattctgaggtgccggacgaccccgaccagcctgatgctgagccggtggagactgatcccgacgccccgggggcccggggactggcgatcggtgactgcgtcgccgacatggaccagctcgacggcaccggcgacatcgacgtcgtcgactgcgccggcccccatgccggcgaggtgtacgcacaggcggatatcgcaggtaagaacctgttccccggcaacgagccgttggggcaggaggcgggagcgatctgcgggggtgactccttcaccggctatgtcggcatcggattccccgagtcctcgctggacgtcgtcacgatgatgccgtccaaggagagctgggcgcaggaggaccggacggtgacctgtgtggtcaccgacccgaacctcgagcagatcgccggcacgctcgagcagagctggcgttagcctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 86 - DNA, 7141 bp, synthetic construct
  source 1..7141: mol_type = other DNA
gtttgtggaagcggtattcgcaatttaattaaagctggtgacaattaatcatcggctcgtataatgtgtggaattgaatcgatataaggaggttaatcatatgcgggagggtggacgccccttccgtgcgcatcgcactctgcccgtcaccgggatcgacgctcaccgcgccggcgtcgaacggcttctcgcgtcctaccgcgcgattcccacggacgccaccgtgcgactcgcgaagaagacgtccaacctgttccgggcgcgggcccagaccagcgcacccggcctcgacgtctccgggctcggcggagtcatctcggtcgacgagcaggaccggaccgcggatgtcgccggaatgtgcacgtacgaagacctggtggacgccaccctcccgtacgggctggcgccgctggtggttccgcaactcaagaccatcacactcggcggcgcggtcaccggcctcggcatcgagtcgacgtcgttccgcaacgggctcccccacgaatcggtcctcgagatcgacgtcctgaccggaagcggcgacatcgtcaccgcgagaccggaaggcgagaactccgacctgttctgggggttccccaactcctacggaaccctcggctactccacccgactgcgcatccagctcgaacccgtcaaacggtatgtggcactgcgccatctgcgtttcgactccctggacgagctgcagtcggcaatggatcgcatcgtcaccgagcgcgtccacgacggcatccccgtcgactatctggacggcgtcgtgttcaccgcgtccgagagttacctgacactgggccatcagaccgacgagggcggccccgtcagcgactacaccgggcagaacatcttctaccggtccatccagcacagttccgtgaaccaccccaaaacggacaaactcaccatccgagactacctgtggcgctgggacaccgactggttctggtgctcgcgcgccttcggcgcccagaaccccaccatccgccggctgtggccgaagaacctcctccgcagcagcttctactggaagctcatcgccctcgaccacaagtacgacatcggcgaccgactcgagaagcgcaagggcaacccgccacgcgaacgcgtcgtgcaggacgtcgaagtgcccatcgagcgcaccgcggacttcgtccgctggttcctcgacgaaatcccgatcgaaccgctgtggctgtgcccgttgcggttgcgggaacctgcccccgccggcgcgtcctcgcaacgcccctggcccctgtaccccctcgaaccgaaacgcacgtacgtgaacatcggattctggtcatcggtgcccatcgttccgggccgacccgagggggccgcgaatcggctgatcgaagacaaggtcagtgacttcgacggacacaagtccctctactccgattcgtactattcacgcgaagatttcgaacgcctctactacggcggcgatcgatacacggaactgaaaaaacgctacgacccgaaatcacgattactggaccttttctccaaggcggtgcaacgtcgatgacaactctgaaagcttcacgctcccaggaccacaagctgaccatcgcagagattctcgaaactctgtccgacggcatgctccccctgcggttctccgcctacgacggcagcgccgccggcccggaggacgccccctacggtctccacctcaagacgacccgaggcaccacctacctggcgaccgcccccggcgacctcggcatggcccgggcctacgtgtccggcgacctcgaggcccgcggcgtccaccccggcgacccgtacgagatcctccgcgtgatgggcgacgaactgcacttccgccgtccgtccgcgctcacgctcgccgccatcacgcgctcgctcggctgggatctgctgcgccccatcgcccctcccccgcaggagcatctcccgcggtggcgtcgagtcgcggaagggttgcggcactccaagtcccgcgacgccgaggtcatccaccaccactacgacgtctcgaacaccttctacgagtatgtcctcggcccgtccatgacgtacacgtgcgcctgctacgagaacgccgagcagaccctcgaagaggcacaggacaacaagtaccgcctcgtcttcgagaagctcggcctccagcccggcgaccgactgctcgacatcggttgcggctggggatcgatggtccggtacgccgcccgccgcggcgtcaaggtcatcggcgccaccctgtcccgagagcaggccgaatgggcacagaaggccatcgccgaagaaggactgtccgacctcgccgaggtccggttctccgactaccgtgacgtccccgagaccggattcgacgccatctcctcgatcggcctgaccgagcacatcggcgtcggcaactaccccgcctacttcggactgctgcagagcaagctccgcgagggcggccggctgctgaaccactgcatcacccggcccgacaaccagagtcaggcacgcgcgggcggcttcatcgaccggtacgtcttccccgacggcgaactcaccggctccggacgcatcatcaccgagatccagaacgtcggactcgaggtgcggcacgaggagaatctgcgcgagcactacgcactcaccctcgccggctggtgccagaacctcgtcgacaactgggacgcctgcgtcgccgaggtcggcgaaggcaccgcacgtgtgtggggtctctacatggccgggtcgcgactgggcttcgaacgcaacgtcgttcagctgcaccaggtcctcgccgtcaagctcggacccaagggcgaggcgcatgtgccgctgcgtccgtggtggaagtagcctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 87 - DNA, 7588 bp, synthetic construct
  source 1..7588: mol_type = other DNA
gtttgtggaagcggtattcgcaatttaattaaagctggtgacaattaatcatcggctcgtataatgtgtggaattgaatcgatataaggaggttaatcatgtgaactgtcagtcttccgcgtccaacctcgccaaccacatcaacgcggtgtacgagctgcgccgcgcctatgcgcggctgtccgccgacaagccggtgcgcctggcgaagaccacctccaacctcttccgcttccgcagccgggacgatgccgcgcgtctcgacgtcagcgctttcacctcggtgatcagcatcgacacggaggcgcgggtcgcggaggtgggcggcatgaccacctacgaggacctggtcgccgccaccctgcggcatggcctgatgccgccggtggttccgcaactgcgcacgatcaccctgggcggtgcggtcaccgggctggggatcgaatcctcgtccttccgcaacgggctcccgcacgagtcagtggaagagatggagatcctcaccggcagcggccaggtggtggtggcccggcgcgacaacgagcaccgcgacctgttctacggtttccccaactcgtacggcaccctcggttacgcgctgcggctccgcatccagctcgaaccggtccgcccctacgtccacctgcggcacctgcggttcaccgatgccgcagcggccatggccgcgctggagcagatctgcgcggaccgcacccacgacggggagaccgtcgacttcgtcgacggcgtcgtgttcgcccgcaacgagctgtacctgaccttggggacgttcaccgaccgggctccgtggaccagcgactacaccggaaccgacatctactaccggtcgatcccccgctacgcgggccccggccccggcgactacctcaccacgcacgactacctgtggcggtgggacaccgactggttctggtgctcccgcgccttcggactgcagcatcccgtggtgcgccgcctgtggccgcgttccttgaaacgctccgacgtctaccgcaagctcgtcgcctgggaccggcgcactgacgcgagccgcctgctcgactactaccgcgggcgcccgcccaaggaaccggtgatccaggacatcgaggttgaggtggggcgggctgccgagttcctcgacttcttccacaccgagatcggcatgtccccggtgtggctgtgcccgctgcggctgcgagaagacacagccgacgatacggaaccggtctggccgctctaccccctcaaaccccgccgcctctacgtcaacttcgggttttggggcctcgttccgatccgtcccggtggaggcaggacataccacaaccggctgatcgaaaaagaagtgacccggttgggcgggcacaagtcgctctactcggacgccttctacgacgaggacgagttctgggagctctacaacggggagatctaccgcaagctcaaagctgcctacgaccccgacggtcgactgctcgacctgtacaccaagtgcgtcggcggcgggtgagaaaggatgagggatgcgactggcggaggtattcgaacgtgtcgtcggacccgatgcgcccgtccacttccgggcctacgacggcagcactgcgggagatccacgcagtgaagtcgctatcgtggttcgccacccggcagccgtcaactacatcgtccaagcgccgggagcactcggtttgacccgcgcctacgtggcgggatacctcgacgtcgaaggggacatgtacaccgcgctgcgggcaatggccgacgtggtgttccaggaccggccgcggctgtcccccggggaactgctgcggatcatccgcgggatcgggtgggtgaagttcgtcaaccggcttccaccgccgccgcaggaggtgcgccagtcccgcctcgccgccctgggctggcgccactccaagcagcgcgacgccgaagccatccagcaccactacgacgtctccaacgccttctacgccctggtcttgggcgagtcgatgacctacacctgcgcggtctacccgaccgagcaggccacgctggagcaggcacagttcttcaagcacgagctgatcgcccgcaagctcggtcttgcccctgggatacgactgctggatgtggggtgcggctggggcggcatggtcatccacgcggcccgggagcacggggtcaaagccctgggggtgaccctgtccaaagagcaggctgagtgggcgcagaagcggatcgcccacgagggcctgggcgacctggcagaagtccggcacatggactaccgggacctgcccgacggcgagtacgacgcgatcagctcgatcgggttgaccgagcacgtcggcaaaaagaacgtgcccgcctacttcgcgtcgctgtaccgcaagctcgtcccgggaggccgcctgctcaaccactgcatcacccggccccgcaacgacctgccgcccttcaaacgcggcggggtgatcaaccgctacgtcttccccgatggggagctggaagggcccggctggctgcaggcggcgatgaacgacgccgggttcgaaatccgccaccaggagaacctgcgggagcactacgcacggaccctgcgggactggctggccaacctggaccgcaactgggatgccgcggtgcgggaagtgggggagggcacggcccgagtgtggcggctctacatggccgggtgcgtgctcggcttcgaacgcaacgtggtgcaactgcaccagatcctcggggtgaagctcgacgggaccgaggcgcggatgccgctgcgccccgacttcgaaccgccgctgccttaaccgcggtgcacagccgggggatatcagtcgcggaaccgggcatgatgagcccatggctgcgaccgatgacgaccggcaccacaccaccgtcgccctcgacctcatcgacgcgtatgtgcgcgccgaccgcagaatgatcggtgaacgttccgcggggatcagcgcggaggcgggggagcggatcgtctccaccctgaaagtgtgcgcggccttccttgcccgccgggtccaggagaccggggtgccgtggcgcgcagcggactcccgggaagcggtcgcccgcaccgtcgccgacctgctggaacccgaggtggaattcgcggtcgtctccgcctgggaggcgtacgcgatcggggagcacgaggccgcctgggtccgggcgcacggcgatccgctggtcttcgtccacatgctggccgcgttctccgctgctatcggcacagcggtctacggccgtgaggagctgctgcccacgctgcgcagggtgacagcacgataacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 88 - DNA, 7074 bp, synthetic construct
  source 1..7074: mol_type = other DNA
gtttgtggaagcggtattcgcaatttaattaaagctggtgacaattaatcatcggctcgtataatgtgtggaattgaatcgatataaggaggttaatcatatgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctgaccggggcggcggcgatgaagacccgcggggcgggacggacaggagggaagcgatgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 89 - DNA, 7331 bp, synthetic construct
  source 1..7331: mol_type = other DNA
gtttgtggaagcggtattcgcaatttaattaaagctggtgacaattaatcatcggctcgtataatgtgtggaattgaatcgatataaggaggttaatcatatgagcggattagttgacccggatagtacttttttaaagaccatcggaaaactgagcaacagcttgtccattggtcgtggagtagatcaaaaagaggtaatccccaaaggctggaacgcccattgggaggcaattacaaagcttaagagaagctttgacgcgattcctgctggggagcgggtgcgtttagctaagaaaacctccaacctgttccgtggacgctccgatgcaggtcacggcctagatgtggcagcgcttgggggagtgattgccattgatccggtcaatgccaccgccgatgtacagggcatgtgcacgtatgaagacctggtagatgccactttaagttatggtctgatgccgttggttgtgcctcaactgaaaaccatcacgcttggtggcgcagtgaccggaatgggcgtggaatccacatccttccgcaacggtttgccacacgaatcagtgctggagatggatatttttaccggcactggtgagatcgtgacttgctcgcccacagaaaatgtcgacctttacagaggttttcccaactcttatggttcgctgggatacgcggtgcggctaaaaattgagctggaaccagtgcaagattacgtccagctgcgccacgtgcgcttcaacgatttagagtctttgaccaaagcgattgaggaagtcgcgtcttctctggagtttgataaccaacccgtcgattaccttgacggcgtggtgttttcacccacggaagcctacttagttcttggcacgcaaacctcacaacctggccccaccagcgattacaccagggatttaagctactaccgctccctgcaacacccagagggcatcacctatgaccgcctgacaatccgcgattacatctggcgctgggacaccgactggttctggtgttcacgcgcattcggcacccaaaaccccgtggtgcgcaaactctggcccagggatctgctgcgctcgagtttctattggaagatcatcggctgggatcgaaaatactccatcgctgatcgcctggaagagcgcaaaggccgcccggctagggaacgggtggtccaagacgtggaagttacgattgataaactgccagaatttttgaaatggttctttgaaagcagcgacatcgagccgctgtggctgtgcccgatcaagcttcgggaggtaccaggtagttcggttggtgctggagaaattttgagctccgctgaagcaatcgactccggtgctgctgaacacccttggccgctgtatcccttgaagaaggacgtgctgtgggtcaacatcggattctggtcctcagtgccggttgatctgatgggctccgatgcaccagagggagcatttaacagagaaatcgaacgcgtcatggcagagctaggcggacataaatcgctgtactccgaagcgttctacaccagggaagactttgaaaaactttatggcggaaccatcccggcgctgctaaaaaagcagtgggatccccacagccgattccccggtttgtatgaaaagacagtaaaaggcgcctaggatcgctcactgtaggtagaggcttgtggtcactacttgtggccacattttaaaaaaatgcacaagaagagaaagcaaagcattatgagtaacgccgtagcgcaggacctcatgaccatcgccgacatcgtcgaggccacgaccactgcacccatcccattccacatcactgccttcgatggaagcttcactggccctgaagatgctccctaccagctgtttgttgccaacacggatgcagtatcctacatcgcaacagcgccaggagatttgggtttggcacgtgcctacctcatgggagacctcatcgtggaaggtgagcatcccggccatccttatgggatctttgatgcgttgaaggagttctaccgctgcttcaaacgcccagatgcatccaccaccttgcagatcatgtggactctgcggaaaatgaatgccttaaaattccaggaaattccaccaatggaacaagcccctgcatggcgtaaagcactgatcaacgggctagcatccaggcactcgaaatcccgcgacaagaaagccattagctaccactacgacgtgggcaatgagttctactccctgtttttagatgattccatgacctatacctgcgcgtattatccaacgccagaatcaagtttggaagaagcccaagaaaacaaataccgcctcatctttgaaaaactgcgtctgaaagaaggcgatcgcctcctagacgtgggatgcggttggggaggcatggtccgctacgccgccaaacacggtgtgaaagccatcggagttacgctgtctgaacagcaatatgagtggggtcaagcagagatcaaacgccaaggtttggaagacctcgcggaaattcgcttcatggattaccgcgatgttccagaaactggattcgatgcgatctcagcaatcggcatcattgaacacatcggtgtgaacaactatcccgactactttgaattgctcagcagcaaactcaaaacaggcggactgatgctcaaccacagcatcacctacccagacaaccgcccccgccacgcaggtgcatttattgatcgctacattttccccgacggtgaactcactggctctggcaccctgatcaagcacatgcaggacaacggtttcgaagtgctgcacgaagaaaacctccgctttgattaccaacgcaccctgcacgcgtggtgcgaaaacctcaaagaaaattgggaggaagcagttgaactcgccggtgaacccactgcacgactctttggcctgtacatggcaggttcggaatggggatttgcccacaacatcgtccagctgcaccaagtactgggtgtgaaactcgatgagcagggaagtcgcggagaagttcctgaaagaatgtggtggactatctaacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 90 - DNA, 7126 bp, synthetic construct
  source 1..7126: mol_type = other DNA
gtttgtggaagcggtattcgcaatttaattaaagctggtgacaattaatcatcggctcgtataatgtgtggaattgaatcgatataaggaggttaatcatgtgtccgctcctgcgaccgatgcacgaaccgcccacgccgacggcgtggagcgattgctcgagagttatcgggcggtgccggcggccgcatcggtgcggctcgccaagcgcacctcgaacctcttccggtcccgagcggcgacggatgcccctggcctcgacacctccggcctgacccacgtcatcgcggtcgaccccggggcgcgcacggccgacgtcgccggcatgtgcacctacgacgacctcgtcgccgcgacactgccgcatgggctcgcgccactcgtggtgccgcaactgaagaccatcaccctcgggggcgccgtaacgggactcggcatcgagtcgacgtcgttccgcaacggtctgccgcacgagtcggtgctcgagatcgacgtgctcaccggcgcaggcgagatcatcacggcgtcgccgatcgagcacgcagagctgttccgcgccttccccaactcgtacggcaccctcggctacgccgtgcgcctgcgcatcgagctcgagccggtcgagccgttcgtcgcactcacgcaccttcggttccatgcgctcaccgacctcatcgaggcaatggagcgcatcatcgagaccggtcgactcgacggggttgccgtcgattccctcgacggcgtggtgttcagcgctgaagagagctacctgtgcgtcggcacgcagaccgcggcatccggcccggtcagcgactacacccgccagcagatcttctatcgctccatccagcatgacgacggtgcgaagcacgaccggctcaccatgcacgactacctgtggcgctgggacgccgactggttctggtgctcgcaggcgttcggcgcgcagcatccgctgattcgccggttctggccgcggcgataccggcgcagccgctcgtactcgacgctcatgcgcctcgaacggcgattcgacctcggcgatcgcctcgagaagctcaagggccggccggcgcgcgaacgcgtgatccaagacgtcgaggtgccgatcgggcgcaccgtcggcttcctcgaatggttcctcgcgaacgtgccgatcgagccgatctggttgtgcccgctgcgcctgcggggcgaccgcggctggcctctctacccgatccggccgcagcagacctacgtcaacatcggcttctggtcgacggttccggtgggcggctccgagggcgagacgaaccgctcgatcgagcgcgccgtgagcgagttcgacggacacaagtcgctgtactccgactcgtactactcgcgcgaggagttcgaggagctctacggcggcgaggcgtaccgggccgtgaagcggcgatacgaccccgactctcgactgctcgacctctatgcgaaggcggtgcaacggcgatgaccacgaccaaacgccaggcgacagcggggcaggctgagaccgcgccgacgacggatgcggcggccgcacccgactcgtcggcgaagctcaccctcgccgagatcctcgagatcgtcgtcgccggtcggctgccgctgaggttcaccgcctacgacgggagctcggcggggccgcctgacgccctgttcggcctcgacctgaagactccgcgaggaacgacctatctcgccaccggccgcggcgatctcggcctcgcccgcgcctacatcgcgggcgacctcgagatacagggggtgcaccccggagacccctacgagctgctcaaggcactcgccgacagcctggtcttcaagctgccaccgccgcgggtgatgacccagatcatccgttcgatcggcgtcgaacatctgcggccgatcgcgccgccgccgcaagaggtgccgccccggtggcgccgcatcgccgaggggctccgacacagcaagggccgcgacgccgaagcgatccaccaccactacgacgtgtcgaacaccttctacgaatgggtgctcgggccgtcgatgacctacacgtgcgcgtgctacccgggcctcgacgcatccctcgacgaggcgcagcagaacaagtaccggctcgtgttcgagaagctgcggctgaagccgggcgaccgactgctcgacgtcggctgcgggtggggcggcatggtgcgctacgccgcgcgccacggcgtgcaggcgttgggcgtgaccctgtcgcgagagcagacggcgtgggcgcagcaggcgatcgccgtcgagggcctcgccgacctcgccgaggtgcgctacggcgactaccgcgacatcgccgaagacggcttcgatgcggtgtcatcgatcgggctgctcgagcacatcggcgtgcgcaactacgcttcgtatttcggctttctgcagtcgcgcttgcggcccgggggactcttgctcaaccactgcatcacccggcccgacaatcgctccgagccgtcggcgcgcggcttcatcgaccggtacgtgttccccgacggagagctcaccggctcgggccgcatcatcaccgaggcgcaggatgtcggcttcgaagtgctgcacgaagagaacctgcgtcagcattatgcactgacactgcgcgattggtgcgccaacctcgtcgcgcactgggaagaggcggtcgccgaggtcgggctgccgaccgcgaaggtgtggggcctctacatggccgggtcacggctcgcgttcgagagcggcggcatccagttgcaccaggtgctggcggtcagaccagacgatcgcagcgacgccgcccagctgccgctgcggccgtggtggacgccatagcctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 91 - DNA, 7505 bp, synthetic construct
  source 1..7505: mol_type = other DNA
gtttgtggaagcggtattcgcaatttaattaaagctggtgacaattaatcatcggctcgtataatgtgtggaattgaatcgatataaggaggttaatcatgtgtctgttgccgtaaccgacgcacgatccgcctacgcccacggcgtgcagcggctggtcgcgagttaccgcgccatccccgccggcgccaccgtccgcctggccaaacccacgtccaacctgttccgcgccagggcgaagagcaccgcggcgggcctcgacacctccggcctgacacatgtgatcgccgtggaccccgagacgcgcaccgccgaggtcgcggggatgtgcacctacgaggacctggtggcggcgacgctgccccacgggctttcaccgctggtggtcccgcaactcaagacgatcaccctcggcggcgccgtcaccgggctcggcatcgagtcggcgtcgttccgcaacggccttccgcacgaatcggtcctggagatggacatcctcaccgggaccggcgacatcgtgcgcgccgcgcccgacgagaatcccgaccttttccgcaccttcccgaattcttatggaacgctgggttactcggttcggctgaagatcgagctggagccggtgaagccgttcgtggcgttacgccatctccgcttccactcactgtcgacactcatcgcgacgatggaccgcatcgtcgacaccgggagtctcgacggtgagcaggtcgactatctcgacggagtggtgttcagcgccgaggagagctacctgtgcgtcggaacacgttccgcgacaccgggtcctgtcagcgactacaccggcgagcacatcttctaccggtcgatccagcacgattgcccgaccgaaggcggacagaagcacgaccggctgacggcgcacgactacttctggcgctgggacaccgactggttctggtgctcaagggcattcggcgcgcagaacccgaaggtccgtcggtggtggccccgacggctccggcgcagcagcttctactggaagctcgtcggctacgaccagcgtttcggcatcgccgaccggatcgagaaacaccacggccggccaccgcgcgaacgcgtcgtccaggacgtcgaggtccccatcgagcgcaccgtcgaattcctgcagtggttcctcgacacgatcccgatagagccgctctggttgtgcccgttgcgacttcgcgatgacaacagctggtcgctgtacccgctccggccccatcgcacgtatgtcaacgtgggattctggtcgtcggtgcccgtcgggccggaggagggtcacaccaacaagctgatcgaacgcaggatcagcgagctggagggacacaagtcgctgtactccgacgccttctattcggccgacgagttcgacgcgctgtacggcggcgagatctaccggaccgtgaagaagacctacgacccagattctcgtttcctcgacctctatgcgaaggcggtgcgacggcaatgacgacttttcgggaacataccgacagttcggcgtccgacccggatcggaaactcactttggcagaggtgttggagatcttcgccgcgggtcgccgtccgctgaagttcaccgcctatgacggaagtagttgcgggcctgaggatgcgacactgggcctcgacctgctgaccccgcggggcacgacctacctggccacggcgccgggtgatctcggcctggcgcgggcctacatcgccggcgatctgcgcctcagtggtgtgcatcccggcgatccccatgacctgctcacggcgctgacggaacgcctggagtacaggcgtccgccggtgcgagtgctggccaatgttctgcgctccatcgggatcgagcacctcaagcccgtcgcgccgccaccccaggagcacctgccgcggtggcggcggatcgcagaggggttgcggcacagcaagacccgtgacgctgaggccatccagcaccactacgacgtctcgaacacgttctactcatgggtcctgggtccgtcgatgacctacacctgcgcctgctatccacacccggatgccacgctggaggaggcgcaggagaacaagtaccggctggtgttcgagaagcttcgactcaagcccggtgaccggctgctcgacgtcggttgcggctggggcggaatggtccgctacgccgcccggcacggggtcaaggtcctgggggtgacgctgtcgaaggagcaggcgcagtgggcggccgacgcagtcgagcgggacggcctgggtgagttggccgaggtccgccacggcgactaccgcgacgtgcgcgagtcgcacttcgacgcagtgtcctcgctcgggctcaccgagcacatcggcgtcgcgaactacccgtcgtacttccgcttcctgaagtcgaaactgcggccgggtggcctgctgctcaaccactgcatcacccgaaacaacaaccggagtcacgccaccgcaggcggattcatcgatcgctatgtctttcccgacggggagctgacggggtcggggcgaatcatcaccgaaatgcaggacgtcggactcgaggtcgtgcacgaggagaatctgcgtcaccactacgcgctgacgctgcgcgactggagccgcaacctggtcgcgcactgggacgacgcggtgaccgaggtcggtctgccgactgccaaggtgtggggcctctacatcgccgcgtcgcgagtcggcttcgagcagaacgccattcagctgcaccaggtgctgtcggtcaagctcgacgagcgtggctcggacggcggactgccgttacgaccctggtggaacgcctagccactatgctctgcccatgatccggttcctgctgcgcatcgcggtctttctgggctcatcagcgatcgggctcctcgtcgccggatggctggtgcccggggtgtcgctgtcggtgtggggcttcgtcacggcagtggtgatcttcaccgtggcgcaggcgatcctgtccccgttcttcctcaagatggccagccgctacgcctcggcgttcctcggcgggatcggtctggtgtcgacgtttgccgcgctgctgctcgtctcgctgctgtccaacggtctgagcatccgcggcatcggatcctggatcgccgcaaccgtggtggtctggttggtgaccgccctggcgacgctggtgctgccgatgttggtgctgcgcgagaagaaaaccgcgtcgcgcgtctgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 92 - DNA, 7123 bp, synthetic construct
  source 1..7123: mol_type = other DNA
gtttgtggaagcggtattcgcaatttaattaaagctggtgacaattaatcatcggctcgtataatgtgtggaattgaatcgatataaggaggttaatcatatgcacgggctgttgtcgaagactagggtatatgtggtgcctgtccttggatctgcactctcggcccacaagtcgggcgttgaccggctgctggcaagctatcgatccattcccgcaacgtccgcggtccggctggccaaaccgacgtcaaacctgttccgcgcccgcaccaaacgtgacgcgcccggcttggacacctcggggctgaccggcgtcctgagcgtggatcccgaaacccgcaccgcggacgtcgccggcatgtgcacctacgcggacctggtggccgcaacgctgccctacggcctgtcgccgctggtcgtcccgcagctgaagaccatcaccctcggcggggcggtcagcggcctggggatcgagtcggcgtcgtttcgcaacgggctgccgcacgaatcggtgctggagatggatatcctcaccggcgctggcgatttgctcaccgcatcacgtacccagcacccggacctgttccgcgccttcccgaattcctatgggacactggggtattcgacccggcttcggatcgagctggaacccgtcgcaccgttcgtcgcgctgcgccacatccgcttccgctcgctgcccgcgctgatcgccgcggccgaacgcatcgtcgacaccggcgggcagggcggaaccccggtcgactacctcgacggggtggtcttcagcgccgacgaaagctacctgtgcgtgggccggcggaccaccacccccggcccggtcagcgactacaccggcaaggacatctactaccagtccatccggcacgacgccccgggcctggaggcgaccaaggatgaccggctgaccatgcacgactacttctggcgctgggacaccgattggttctggtgctcgcgcgcgttcggcgtgcaggacccgcgggtgcgacgcttctggccgcgccgttatcggcgcagcagcttctactggaagctgatttccctggaccggcgcttcgggatctccgaccgcatcgaggcgcgcaacgggcggcccccacgcgaacgggtggtgcaagacatcgagattccaatcgaacggacctgcgacttcctggagtggttcctggacaacgtgccaatcacgccgatctggttgtgcccgttgcgccttcgcgaccgcgacggctggccgttgtacccgatgcggccggatcacacgtacgtcaacgtcggcttctggtcgtcggtgccggggggcgcgaccgagggcgccgccaaccggatgatcgaagaaaaggtgagcgaactcgacgggcacaagtccctgtactccgattccttctactcccgcgaggacttcgacgagctgtacggcggcgagacctacaacaccgtcaagaaaacctacgaccccgattctcgtttactcgacctctacgcaaaggcggtgcaacggcgatgacgactaccaaggaaccccaccgcacgtcgcacgggaaactgagcatggccgagatcctggaggtcttcgccgccaccggccgacatccgctgaagttcaccgcctacgacggcagcatcgccggcaacgaggacgccgaactgggcctggaccttcgcagcccccgcggcgccacctatctggcgaccgcccccggcgaactcggcctcgcccgcgcctacgtgtcgggcgacctgcaggcctacggcgtccatcccggcgacccgtaccaactgctcaagacgctcaccgatcgggtggaattcaagcggcccccggtgcgggtgctggccaacgtcgtgcggtcgctggggttcgagcggttgctgccggtcgcgccgcccccgcaggaggcgctgccccggtggcggcgcatcgccgacgggctgatgcacacgaggacccgcgacgccgaggccatccaccaccactacgacgtgtccaacaccttctacgaattggtgttggggccgtcgatgacctacacctgcgcggtgtatcccgatgccgacgcgacactcgaacaggcgcaggagaacaagtaccggctgatcttcgagaagctgcggctgaaggcgggcgaccggctgctcgacgtcggctgcggctggggcggcatggtgcgctacgcggcccggcgcggcgtccgggccaccggcgccaccctgtcggccgaacaggcgaagtgggcgcagaaggcgatcgccgaggaaggccttgcggacctggccgaggtgcgccacaccgactatcgggacgtgggcgaggcggcgttcgacgccgtgtcctcgatcgggctgaccgagcacatcggcgtcaagaattaccccgcctacttcggcttcttgaagtcgaagctgcgcaccggcggcctgctgctcaatcactgcatcacccgccacgacaacacgtcgacgtcgttcgcgggcggattcaccgatcgctatgtcttcccggacggggagctgaccggctcgggccgcatcacctgcgacgtccaggactgcggcttcgaggtgctgcacgcggagaacttccgccaccactacgcgatgacgctgcgcgactggtgccgcaatctggtcgagaactgggacgccgcggtcagcgaggtcggcctaccgaccgcgaaggtctggggcctgtacatggcggcgtcacgggttgcgttcgagcagaacaaccttcagctgcatcacgtgctggcggccaagaccgacgcgcggggcgacgacgacctgccgctgcggccgtggtggacggcctgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 93 - DNA, 9807 bp, synthetic construct
  source 1..9807: mol_type = other DNA
ggttatatagggatatagcacagagatatatagcaaagagatacttttgagcaatgtttgtggaagcggtattcgcaatttaattaacgcttaccttggccgttagacatcatggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccctgctctgcgaggctggccggctaccgccggcgtaacagatgagggcaagcggatggctgatgaaaccaagccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgagtttaaacagctggtgacaattaatcatcggctcgtataatgtgtggaattgaatcgatataaggaggttaatcatgtgtctgtggttactactgacgcacaggctgcccatgccgccggcgtctcgcgtcttctggccagctaccgggcgatcccgcccagcgcgacagtgcgccttgcgaaaccgacgtccaacctgttccgcgcccgcgcccgcaccaatgtgaagggtctcgacgtctcgggcctgaccggtgtgatcggtgtcgacccggacgcgcgcaccgccgatgtggcgggcatgtgcacctacgaggacctggtggcggccacgcttccgtacggccttgccccactggtggtgccgcagctcaagaccatcacgctcggtggcgcggtcaccggtctgggcatcgagtccacgtcgttccgcaacggtctgccgcacgaaagtgtcctggagatggacatcttgaccggttcgggcgagatcgtcacggcctcaccggatcagcactcggatctgttccatgcgttccccaattcatatggaacccttggttattccacccggctgcgcatcgaactggagcccgtgcacccgtttgtggcgttgcgccacctgcgctttcactcgatcaccgatctggtcgcggcgatggaccggatcatcgagaccggcgggctggacggtgaacccgtcgactacctcgacggcgtggtgttcagcgcgactgagagttacctgtgtgttggcttcaagacgaaaacgccggggccggtcagcgattacacaggtcagcagatcttctaccggtcgatccagcatgacggcgacaccggcgccgagaaacacgaccggctgaccatccacgactacctgtggcgctgggacaccgactggttctggtgctcacgggcattcggcgctcagcatccggtgatccgcaggttctggccgcggcggctgcgccgcagcagcttctactggaagctggtggcctacgaccagcggtacgacatcgccgaccgtatcgagaagcgcaacgggcgcccgccgcgcgagcgggtggtccaggacgtcgaggtgcccatcgagcggtgcgcggacttcgtcgagtggttcctgcagaatgtgccgatcgagccgatctggctgtgccccctacggttgcgtgacagcgccgacggcggtgcctcgtggcccctgtatccgctgaaggcgcaccacacctacgtcaacatcggtttctggtcatcagtgccggtgggccccgaggagggccacaccaaccgcctcatcgagaaaaaagtcgcggagctggacgggcacaaatctttgtactcggacgcttattacacacgtgacgaattcgacgagctgtacggcggtgaggtctacaacaccgtcaagaagacgtacgacccggattcacgtctgctagacctgtattcgaaggcggtgcaaagacaatgaccacattcaaagaacgcgagacgtccacagcggaccgcaagctcaccctggccgagatcctcgagatcttcgccgcgggtaaggagccgctgaagttcactgcgtacgacggcagctcggccggtcccgaggacgccacgatgggtctggacctcaagaccccgcgtgggaccacctatctggccacggcacccggcgatctgggcctggcccgtgcgtatgtctccggtgacctggagccgcacggcgtgcatcccggcgatccctacccgctgctgcgcgccctggccgaacgcatggagttcaagcgcccgcctgcgcgtgtgctggcgaacatcgtgcgctccatcggcatcgagcacctcaagccgatcgcaccgccgccgcaggaggcgctgccccggtggcgccgcatcatggagggcctgcggcacagcaagacccgcgacgccgaggccatccaccaccactacgacgtgtcgaacacgttctacgagtgggtgctgggcccgtcgatgacctacacgtgcgcgtgctaccccaccgaggacgcgaccctcgaagaggcccaggacaacaagtaccgcctggtgttcgagaagctgcgcctgaagcccggtgaccggttgctcgacgtgggctgcggctggggcggcatggtccgctacgcggcccgccacggcgtcaaggcgctcggtgtcacgctcagccgcgaacaggcgacgtgggcgcagaaggccatcgcccaggaaggtctcaccgatctggccgaggtgcgtcacggtgattaccgcgacgtcatcgaatccgggttcgacgcggtgtcctcgatcgggctgaccgagcacatcggcgtgcacaactacccggcgtacttcaacttcctcaagtcgaagctgcgcaccggtggcctgctgctcaaccactgcatcacccgcccggacaaccggtcggcgccatcggccggcgggttcatcgacaggtacgtgttccccgacggggagctcaccggctcgggccgcatcatcaccgaggcccaggacgtgggccttgaggtgatccacgaggagaacctacgcaatcactatgcgatgacgctgcgcgactggtgccgcaacctggtcgagcactgggacgaggcggtcgaagaggtcgggctgcccaccgcgaaggtgtggggcctgtacatggccggctcacgtctgggcttcgagaccaatgtggttcagctgcaccaggttctggcggtcaagcttgacgatcagggcaaggacggcggactgccgttgcggccctggtggtccgcctagcctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggta

SEQ ID NO: 94 - DNA, 10293 bp, synthetic construct
  source 1..10293: mol_type = other DNA
gtttgtggaagcggtattcgcaatttaattaacgcttaccttggccgttagacatcatggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccctgctctgcgaggctggccggctaccgccggcgtaacagatgagggcaagcggatggctgatgaaaccaagccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgagtttaaacagctggtgacaattaatcatcggctcgtataatgtgtggaattgaatcgatataaggaggttaatcatgtgaactgtcagtcttccgcgtccaacctcgccaaccacatcaacgcggtgtacgagctgcgccgcgcctatgcgcggctgtccgccgacaagccggtgcgcctggcgaagaccacctccaacctcttccgcttccgcagccgggacgatgccgcgcgtctcgacgtcagcgctttcacctcggtgatcagcatcgacacggaggcgcgggtcgcggaggtgggcggcatgaccacctacgaggacctggtcgccgccaccctgcggcatggcctgatgccgccggtggttccgcaactgcgcacgatcaccctgggcggtgcggtcaccgggctggggatcgaatcctcgtccttccgcaacgggctcccgcacgagtcagtggaagagatggagatcctcaccggcagcggccaggtggtggtggcccggcgcgacaacgagcaccgcgacctgttctacggtttccccaactcgtacggcaccctcggttacgcgctgcggctccgcatccagctcgaaccggtccgcccctacgtccacctgcggcacctgcggttcaccgatgccgcagcggccatggccgcgctggagcagatctgcgcggaccgcacccacgacggggagaccgtcgacttcgtcgacggcgtcgtgttcgcccgcaacgagctgtacctgaccttggggacgttcaccgaccgggctccgtggaccagcgactacaccggaaccgacatctactaccggtcgatcccccgctacgcgggccccggccccggcgactacctcaccacgcacgactacctgtggcggtgggacaccgactggttctggtgctcccgcgccttcggactgcagcatcccgtggtgcgccgcctgtggccgcgttccttgaaacgctccgacgtctaccgcaagctcgtcgcctgggaccggcgcactgacgcgagccgcctgctcgactactaccgcgggcgcccgcccaaggaaccggtgatccaggacatcgaggttgaggtggggcgggctgccgagttcctcgacttcttccacaccgagatcggcatgtccccggtgtggctgtgcccgctgcggctgcgagaagacacagccgacgatacggaaccggtctggccgctctaccccctcaaaccccgccgcctctacgtcaacttcgggttttggggcctcgttccgatccgtcccggtggaggcaggacataccacaaccggctgatcgaaaaagaagtgacccggttgggcgggcacaagtcgctctactcggacgccttctacgacgaggacgagttctgggagctctacaacggggagatctaccgcaagctcaaagctgcctacgaccccgacggtcgactgctcgacctgtacaccaagtgcgtcggcggcgggtgagaaaggatgagggatgcgactggcggaggtattcgaacgtgtcgtcggacccgatgcgcccgtccacttccgggcctacgacggcagcactgcgggagatccacgcagtgaagtcgctatcgtggttcgccacccggcagccgtcaactacatcgtccaagcgccgggagcactcggtttgacccgcgcctacgtggcgggatacctcgacgtcgaaggggacatgtacaccgcgctgcgggcaatggccgacgtggtgttccaggaccggccgcggctgtcccccggggaactgctgcggatcatccgcgggatcgggtgggtgaagttcgtcaaccggcttccaccgccgccgcaggaggtgcgccagtcccgcctcgccgccctgggctggcgccactccaagcagcgcgacgccgaagccatccagcaccactacgacgtctccaacgccttctacgccctggtcttgggcgagtcgatgacctacacctgcgcggtctacccgaccgagcaggccacgctggagcaggcacagttcttcaagcacgagctgatcgcccgcaagctcggtcttgcccctgggatacgactgctggatgtggggtgcggctggggcggcatggtcatccacgcggcccgggagcacggggtcaaagccctgggggtgaccctgtccaaagagcaggctgagtgggcgcagaagcggatcgcccacgagggcctgggcgacctggcagaagtccggcacatggactaccgggacctgcccgacggcgagtacgacgcgatcagctcgatcgggttgaccgagcacgtcggcaaaaagaacgtgcccgcctacttcgcgtcgctgtaccgcaagctcgtcccgggaggccgcctgctcaaccactgcatcacccggccccgcaacgacctgccgcccttcaaacgcggcggggtgatcaaccgctacgtcttccccgatggggagctggaagggcccggctggctgcaggcggcgatgaacgacgccgggttcgaaatccgccaccaggagaacctgcgggagcactacgcacggaccctgcgggactggctggccaacctggaccgcaactgggatgccgcggtgcgggaagtgggggagggcacggcccgagtgtggcggctctacatggccgggtgcgtgctcggcttcgaacgcaacgtggtgcaactgcaccagatcctcggggtgaagctcgacgggaccgaggcgcggatgccgctgcgccccgacttcgaaccgccgctgccttaaccgcggtgcacagccgggggatatcagtcgcggaaccgggcatgatgagcccatggctgcgaccgatgacgaccggcaccacaccaccgtcgccctcgacctcatcgacgcgtatgtgcgcgccgaccgcagaatgatcggtgaacgttccgcggggatcagcgcggaggcgggggagcggatcgtctccaccctgaaagtgtgcgcggccttccttgcccgccgggtccaggagaccggggtgccgtggcgcgcagcggactcccgggaagcggtcgcccgcaccgtcgccgacctgctggaacccgaggtggaattcgcggtcgtctccgcctgggaggcgtacgcgatcggggagcacgaggccgcctgggtccgggcgcacggcgatccgctggtcttcgtccacatgctggccgcgttctccgctgctatcggcacagcggtctacggccgtgaggagctgctgcccacgctgcgcagggtgacagcacgataacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaat

SEQ ID NO: 95 - DNA, 5654 bp, synthetic construct
  source 1..5654: mol_type = other DNA
tgggtaggttatatagggatatagcacagagatatatagcaaagagatacttttgagcaatgtttgtggaagcggtattcgcaatttaattaaagctggtgacaattaatcatcggctcgtataatgtgtggaattgaatcgatataaggaggttaatcatatgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtgga

SEQ ID NO: 96 - DNA, 5759 bp, synthetic construct
  source 1..5759: mol_type = other DNA
tgggtaggttatatagggatatagcacagagatatatagcaaagagatacttttgagcaatgtttgtggaagcggtattcgcaatttaattaaagctggtgacaattaatcatcggctcgtataatgtgtggaattgaatcgatataaggaggttaatcatatgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctgacctcaaaatatattttccctctatcttctcgttgcgcttaatttgactaattctcattagcgaggcgcgcctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacagcgatcgcgcggccgcgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacaccacatatgcgtatatataccaatctaagtctgtgctccttccttcgttcttccttctgctcggagattaccgaatcaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtgga

SEQ ID NO: 97 - DNA, 2664 bp, Thermomonospora curvata
  source 1..2664: mol_type = genomic DNA
atgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgcgctggtggtgccgagggtggcaatggcggtggcgccatgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtga

SEQ ID NO: 98 - DNA, 2664 bp, Thermomonospora curvata
  source 1..2664: mol_type = genomic DNA
atgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtggctggtggtgccgagggtggcaatggcggtggcgccatgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctga

SEQ ID NO: 99 - protein, 12 aa, synthetic construct
AGGAEGGNGGGA

SEQ ID NO: 100 - protein, 382 aa, Escherichia coli
MSSSCIEEVSVPDDNWYRIANELLSRAGIAINGSAPADIRVKNPDFFKRVLQEGSLGLGESYMDGWWECDRLDMFFSKVLRAGLENQLPHHFKDTLRIAGARLFNLQSKKRAWIVGKEHYDLGNDLFSRMLDPFMQYSCAYWKDADNLESAQQAKLKMICEKLQLKPGMRVLDIGCGWGGLAHYMASNYDVSVVGVTISAEQQKMAQERCEGLDVTILLQDYRDLNDQFDRIVSVGMFEHVGPKNYDTYFAVVDRNLKPEGIFLLHTIGSKKTDLNVDPWINKYIFPNGCLPSVRQIAQSSEPHFVMEDWHNFGADYDTTLMAWYERFLAAWPEIADNYSERFKRMFTYYLNACAGAFRARDIQLWQVVFSRGVENGLRVAR

SEQ ID NO: 101 - protein, 499 aa, Escherichia coli
MSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF